(19) United States
(12) Patent Application Publication
(10) Pub. No.: US 2006/0189650 A1
(43) Pub. Date: Aug. 24, 2006

(54) Novel piperidine derivatives as modulators of chemokine receptor ccr5

(76) Inventors: John Cumming, Cheshire (GB); Alan Faull, Cheshire (GB); Colin Fielding, Cheshire (GB); John Oldfield, Cheshire (GB); Howard Tucker, Cheshire (GB)

Correspondence Address: FISH & RICHARDSON P.C., P.O BOX 1022, MINNEAPOLIS, MN, 55440-1022, US

(21) Appl. No.: 10/539,859

(22) PCT Filed: Dec. 18, 2003
(86) PCT No.: PCT/SE03/02008

§ 371 Date: Jun. 17, 2005

(30) Foreign Application Priority Data
Dec. 20, 2002 (SE) 0203821.4
Feb. 24, 2003 (SE) 0300499.1
May 15, 2003 (SE) 0301425.5

(51) Int. Cl. A61K 31/46; A61K 31/454; A61K 31/445; C07D 211/26; C07D 401/02

(52) U.S. Cl. 514/304; 514/317; 514/326; 546/125; 546/207; 546/229; 546/236

(57) ABSTRACT

Compounds of formula (I) wherein R¹, R²R³, R⁴, Λ, X, m and n are as defined; compositions comprising them, processes for preparing them and their use in medical therapy them and their use in medical therapy (for example mod ulating CCR5 receptor activity in a warm blooded animal).

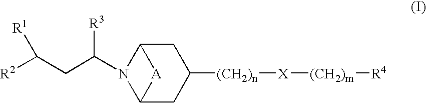

The present invention relates to heterocyclic derivatives having pharmaceutical activity, to processes for preparing such derivatives, to pharmaceutical compositions comprising such derivatives and to the use of such derivatives as active therapeutic agents.

Pharmaceutically active piperidine derivatives are disclosed in WO01/87839, EP-A1-1013276, WO00/08013, WO99/38514, WO99/04794, WO0076511, WO00/76512, WO00/76513, WO00/76514, WO00/76972, US 2002/0094989 and Bioorg. Med. Chem. Lett. 13 (2003) 119-123.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating-peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

The CCR5 receptor is expressed on T-lymphocytes, monocytes, macrophages, dendritic cells, microglia and other cell types. These detect and respond to several chemokines, principally “regulated on activation normal T-cell expressed and secreted” (RANTES), macrophage inflammatory proteins (MIP) MIP-1α and MIP-1β and monocyte chemoattractant protein-2 (MCP-2).

This results in the recruitment of cells of the immune system to sites of disease. In many diseases it is the cells expressing CCR5 which contribute, directly or indirectly, to tissue damage. Consequently, inhibiting the recruitment of these cells is beneficial in a wide range of diseases.

CCR5 is also a co-receptor for HIV-1 and other viruses, allowing these viruses to enter cells. Blocking the receptor with a CCR5 antagonist or inducing receptor internalisation with a CCR5 agonist protects cells from viral infection.

The present invention provides a compound of formula (I): 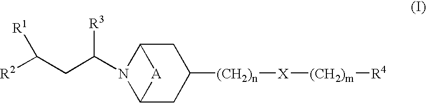
wherein:
A is absent or is (CH₂)₂;
R¹ is C₁₋₈ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl, aryl or heteroaryl;
R¹⁰, R¹³, R¹⁵, R¹⁶ and R¹⁸ are hydrogen or C₁₋₆ alkyl;
R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₆ alkyl), halo or C₁₋₄ alkyl); or R¹¹, R¹², R¹⁴ and R¹⁷ can also be hydrogen;
or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, S(O)₁(C₁₋₆ alkyl) or C(O)(C₁₋₆ alkyl);
R² is C₁₋₆ alkyl, phenyl, heteroaryl or C₃₋₇ cycloalkyl;
R³ is H or C₁₋₄ alkyl;
R⁴ is aryl, heteroaryl, C₁₋₆ alkyl or C₃₋₇ cycloalkyl;
X is O or S(O)ₚ;
m and n are, independently, 0, 1, 2 or 3, provided m+n is 1 or more;
aryl, phenyl and heteroaryl moieties are independently optionally substituted by one or more of halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy (optionally mono-substituted by CO₂R⁵³, C(O)NR⁵⁴R⁵⁵, cyano, heteroaryl or C(O)NHS(O)₂R⁵⁶), NHC(O)NHR⁵⁷, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl (which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen;
k, l, p and q are, independently, 0, 1 or 2;
R²⁰, R²², R²⁴, R²⁶, R²⁷, R²⁹, R³¹, R³³, R³⁷, R⁴⁰, R⁵¹ and R⁵⁴ are, independently, hydrogen or C₁₋₆ alkyl;
R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰, R⁵², R⁵³, R⁵⁵, R⁵⁶ and R⁵⁷ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; R²¹, R²³, R²⁵, R²⁸, R³⁰, R³⁴, R³⁵, R³⁶, R⁴¹, R⁴², R⁴³, R⁴⁵, R⁴⁶, R⁴⁷, R⁵², R⁵³, R⁵⁵ and R⁵⁷ may additionally be hydrogen;
or a pharmaceutically acceptable salt thereof or a solvate thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, hydrobromide, phosphate, acetate, fumarate, maleate, tartrate, citrate, oxalate, methanesulphonate or p-toluenesulphonate. In addition to these further examples of acid addition salts are succinate, glutarate or malonate.

The compounds of the invention may exist as solvates (such as hydrates) and the present invention covers all such solvates.

Alkyl groups and moieties are straight or branched chain and, for example, comprise one to six (such as one to four) carbon atoms. Alkyl is, for example, methyl, ethyl, n-propyl, iso-propyl, n-butyl, sec-butyl or tert-butyl. Methyl is sometimes abbreviated to Me hereinbelow.

Haloalkyl includes CF₃, and haloalkoxy includes CF₃.

Fluoroalkyl includes, for example, one to six, such as one to three, fluorine atoms, and comprises, for example, a CF₃ group. Fluoroalkyl is, for example, CF₃ or CH₂CF₃—

Cycloalkyl is, for example, cyclopropyl, cyclopentyl or cyclohexyl (such as cyclohexyl). Cycloalkenyl includes cyclopentenyl.

Heterocyclyl is, for example, piperidine, piperazine, pyrrolidine, azetidine, tetrahydrofuran, morpholine or thiomorpholine. Further examples of heterocyclyl are tetrahydropyran and tetrahydrothiopyran.

Aryl includes phenyl and naphthyl. In one aspect of the invention aryl is phenyl.

Heteroaryl is, for example, an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, pyridinyl, pyrimidinyl, pyrazinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl, a naphthyridinyl (for example [1,6]naphthyridinyl or [1,8]naphthyridinyl), a benzothiazinyl or dibenzothiophenyl (also known as dibenzothienyl); or an N-oxide thereof, or an S-oxide or S-dioxide thereof A further example of heteroaryl is tetrazolyl.

Aryloxy includes phenoxy.

Heteroaryloxy includes pyridinyloxy and pyrimidinyloxy.

Phenyl(C₁₋₄ alkyl)alkyl is, for example, benzyl, 1-(phenyl)eth-1-yl or 1-(phenyl)eth-2-yl.

Heteroaryl(C₁₋₄ alkyl)alkyl is, for example, pyridinylmethyl, pyrimidinylmethyl or 1-(pyridinyl)eth-2-yl.

Phenyl(C₁₋₄ alkoxy) is, for example, benzyloxy or phenylCH(CH₃)O.

Heteroaryl(C₁₋₄ alkoxy) is, for example, pyridinylCH₂O, pyrimidinylCH₂O or pyridinylCH(CH₃)O.

Heteroaryl rings can carry various substituents including sulphonyl groups. A sulphonyl group on a heteroaryl ring can be a good leaving group (susceptible to nucleophilic displacement) and examples of such situation are: 2-methanesulphonyl-pyridine and 2- or 4-methanesulphonyl-pyrimidine. The present invention covers compounds including a heteroaryl ring carrying a sulphonyl group which are sufficiently stable (non-reactive) to be isolated using the experimental procedures described.

In one particular aspect the present invention provides a compound of formula (I) wherein: A is absent or is (CH₂)₂; R¹ is C₁₋₈ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl, aryl or heteroaryl; R¹⁰, R¹³, R¹⁵, R¹⁶ and R¹⁸ are hydrogen or C₁₋₆ alkyl; R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₆ alkyl), halo or C₁₋₄ alkyl); or R¹¹, R¹², R¹⁴ and R¹⁷ can also be hydrogen; or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, S(O)₁(C₁₋₆ alkyl) or C(O)(C₁₋₆ alkyl); R² is C₁₋₆ alkyl, phenyl, heteroaryl or C₃₋₇ cycloalkyl; R³ is H or C₁₋₄ alkyl; R⁴ is aryl, heteroaryl, C₁₋₆ alkyl or C₃₋₇ cycloalkyl; X is O or S(O)ₚ; m and n are, independently, 0, 1, 2 or 3, provided m+n is 1 or more; aryl, phenyl and heteroaryl moieties are independently optionally substituted by one or more of halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl C₁₋₆ haloalkyl, C₁₋₆ alkoxy(C₁₋₆)alkyl, C₁₋₆ alkoxy, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃; unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl }optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen; k, l, p and q are, independently, 0, 1 or 2; R²⁰, R²², R²⁴, R²⁶, R²⁷, R²⁹, R³¹, R³³, R³⁷, R⁴⁰ and R⁵¹ are, independently, hydrogen or C₁₋₆ alkyl; R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰ and R⁵² are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃;
R²¹, R²³, R²⁵, R²⁸, R³⁰, R³⁴, R³⁵, R³⁶, R⁴¹, R⁴², R⁴³, R⁴⁵, R⁴⁶, R⁴⁷ and R⁵² may additionally be hydrogen; or a pharmaceutically acceptable salt thereof or a solvate thereof.

In a further aspect the present invention provides a compound of formula (I) wherein: A is absent or is (CH₂)₂; R¹ is C₁₋₈ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl (for example piperidine, piperazine, pyrrolidine or azetidine), aryl or heteroaryl; R¹⁰, R¹³, R¹⁵, R¹⁶ and R¹⁸ are hydrogen or C₁₋₆ alkyl; R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₆ alkyl), halo or C₁₋₄ alkyl); or R¹¹, R¹², R¹⁴ and R¹⁷ can also be hydrogen; or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, S(O)₁(C₁₋₆ alkyl) or C(O)(C₁₋₆ alkyl); R² is C₁₋₆ alkyl, phenyl, heteroaryl or C₃₋₇ cycloalkyl; R³ is H or C₁₋₄ alkyl; R⁴ is aryl, heteroaryl, C₁₋₆ alkyl or C₃₋₇ cycloalkyl; X is O or S(O)ₚ; m and n are, independently, 0, 1, 2 or 3, provided m+n is 1 or more; aryl, phenyl and heteroaryl moieties are independently optionally substituted by one or more of halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃; unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen; k, l, p and q are, independently, 0, 1 or 2; R²⁰, R²², R²⁴, R²⁶, R²⁷, R²⁹, R³¹, R³³, R³⁷, R⁴⁰ and R⁵¹ are, independently, hydrogen or C₁₋₆ alkyl; R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰ and R⁵² are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; R²¹, R²³, R²⁵, R²⁸, R³⁰, R³⁴, R³⁵, R³⁶, R⁴¹, R⁴², R⁴³, R⁴⁴, R⁴⁵, R⁴⁶, R⁴⁷ andR⁵² may additionally be hydrogen; or a pharmaceutically acceptable salt thereof or a solvate thereof.

In another aspect the present invention provides a compound of formula (I) wherein A is absent or is (CH₂)₂; R¹ is C₁₋₈ alkyl, C(O)₂R¹², NR¹³C(O)R¹⁴, NR¹⁵C(O)NR¹⁶R¹⁷, NR¹⁸C(O)₂R¹⁹, heterocyclyl (for example piperidine, piperazine, pyrrolidine or azetidine), aryl or heteroaryl; R¹⁰, R¹³, R¹⁵, R¹⁶ and R¹⁸ are hydrogen or C₁₋₆ alkyl; R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₆ alkyl), halo or C₁₋₄ alkyl); or R¹¹, R¹², R¹⁴ and R¹⁷ can also be hydrogen; or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, S(O)₁(C₁₋₆ alkyl) or C(O)(C₁₋₆ alkyl); R² C₁₋₆ alkyl, phenyl, heteroaryl or C₃₋₇ cycloalkyl; R³ is H or C₁₋₄ alkyl; R⁴ is aryl or heteroaryl; X is O or S(O)ₚ; m and n are, independently, 0, 1, 2 or 3, provided m+n is 1 or more, and provided that when X is O then m and n are not both 1; unless specified otherwise aryl, phenyl and heteroaryl moieties are independently optionally substituted by one or more of halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³² C(O)NR³³R³⁴, CO₂R³⁶, NR³⁷CO₂R³⁸, S(O)_{q}R³⁹, C₁₋₆ alkyl, C₂₋₆ alkenyl, C₂₋₆ alkynyl, C₃₋₁₀-cycloalkyl, C₁₋₆ haloalkyl, C₁₋₆ alkoxy(C₁₋₆)alkyl, C₁₋₆ alkoxy, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃; unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {of which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl) (such as tert-butoxycarbonyl), C(O)₂(phenyl(C₁₋₂ alkyl)) (such as benzyloxycarbonyl), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen; k, l, p and q are, independently, 0, 1 or 2; R²⁰, R²², R²⁴, R²⁶, R²⁷, R²⁹, R³¹, R³³, R³⁷ and R⁴⁰ are, independently, hydrogen or C₁₋₆ alkyl; R²¹, R²³, R²⁵, R²⁸, R³⁰, R³², R³⁴, R³⁸, R³⁹, R⁴¹, R⁴², R⁴³, R⁴⁴, R⁴⁵, R⁴⁶, R⁴⁷ and R⁴⁸ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; R²¹, R²³, R²⁵, R²⁸, R³⁰, R³⁴, R³⁵, R³⁶, R⁴¹, R⁴², R⁴³, R⁴⁵, R⁴⁶ and R⁴⁷ may additionally be hydrogen; or a pharmaceutically acceptable salt thereof or a solvate thereof.

In a further aspect the present invention provides a compound of formula (I) wherein, unless specified otherwise, aryl, phenyl and heteroaryl moieties are independently optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, phenyl, heteroaryl, phenyl(C₁₋₄ alkyl), heteroaryl(C₁₋₄ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂H, CO₂(C₁₋₄ alkyl), NHC(O)(C₁₋₄ alkyl), NHS(O)₂(C₁₋₄ alkyl), CF₃ or OCF₃.

In yet another aspect the present invention provides a compound of formula (I) wherein, unless specified otherwise, aryl, phenyl and heteroaryl moieties are independently optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, C(O)[N-linked heterocyclyl], CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, phenyl, heteroaryl, phenyl(C₁₋₄ alkyl), heteroaryl(C₁₋₄ alkyl), NHC(O)phenyl, NHC(O)heteroaryl, NHC(O)(C₁₋₄ alkyl)phenyl, NHC(O)(C₁₋₄ alkyl)heteroaryl, NHS(O)₂phenyl, NHS(O)₂heteroaryl, NHS(O)₂(C₁₋₄ alkyl)phenyl, NHS(O)₂(C₁₋₄ alkyl)heteroaryl, NHC(O)NH(C₁₋₆ alkyl), NHC(O)NH(C₃₋₇ cycloalkyl), NHC(O)NHphenyl, NHC(O)NHheteroaryl, NHC(O)NH(C₁₋₄ alkyl)phenyl or NHC(O)NH(C₁₋₄ alkyl)heteroaryl; wherein the foregoing phenyl and heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃.

In a further aspect the present invention provides a compound of formula (I) wherein, unless specified otherwise, aryl, phenyl and heteroaryl moieties are independently optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl group (such as tetrazolyl) are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃ {and in a further aspect of the invention the foregoing heteroaryl groups (such as tetrazolyl) are optionally substituted by C₁₋₄ alkyl}.

In another aspect the present invention provides a compound of formula (I) wherein, unless specified otherwise, aryl, phenyl and heteroaryl moieties are independently optionally substituted by one or more of halo, hydroxy, nitro, S(C₁₋₄ alkyl), S(O)(C₁₋₄ alkyl), S(O)₂(Cᵢ₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), CO₂H, CO₂(C₁₋₄ alkyl), NHC(O)(C₁₋₄ alkyl), NHS(O)₂(C₁₋₄ alkyl), CF₃, CHF₂, CH₂F, CH₂CF₃ or OCF₃.

In a further aspect of the invention heteroaryl is tetrazolyl, pyrrolyl, thienyl, imidazolyl, thiazolyl, isoxazolyl, pyridinyl, pyrimidinyl, pyrazinyl or quinolinyl. In a still further aspect heteroaryl is pyrrolyl, thienyl, imidazolyl, thiazolyl, isoxazolyl, pyridinyl, pyrimidinyl, pyrazinyl or quinolinyl.

In another aspect of the invention R¹⁰, R¹³, R¹⁵, R¹⁶ and R¹⁸ are hydrogen or C₁₋₄ alkyl (for example methyl). In yet another aspect R¹⁰, R¹³, R¹⁵, R¹⁶ and R¹⁸ are hydrogen.

In a further aspect of the invention R¹¹, R¹², R¹⁴, R¹⁷, R¹⁸ and R¹⁹ are C₁₋₈ alkyl (optionally substituted by halo, C₁₋₆ alkoxy, C₁₋₆ haloalkoxy, C₃₋₆ cycloalkyl (optionally substituted by halo), C₅₋₆ cycloalkenyl, S(O)₂(C₁₋₄ alkyl), heteroaryl, phenyl, heteroaryloxy or aryloxy (for example phenoxy)), phenyl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)ₖ(C₁₋₄ alkyl), halo or C₁₋₄ alkyl); k is 0, 1 or 2; or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl or C(O)(C₁₋₆ alkyl).

In yet another aspect of the invention R¹¹, R¹², R¹⁴, R¹⁷ and R¹⁹ are C₁₋₈ alkyl (optionally substituted by halo (such as fluoro)), phenyl (optionally substituted as recited above), C₃₋₆ cycloalkyl (optionally substituted by halo (such as fluoro)) or C-linked nitrogen containing heterocyclyl (optionally substituted on the ring nitrogen).

In a further aspect R¹ is NHC(O)R¹⁴, phenyl or heterocyclyl, wherein R¹⁴ is as defined above, and phenyl and heterocyclyl are optionally substituted as described above.

In another aspect of the invention R¹ is NR¹³C(O)R¹⁴, wherein R¹³ and R¹⁴ are as defined above. For example R¹³ is hydrogen.

In yet another aspect of the invention R¹⁴ is C₁₋₈ alkyl (optionally substituted by halo (such as fluoro, for example to form CF₃CH₂)), phenyl (optionally substituted as recited above), C₃₋₆ cycloalkyl (optionally substituted by halo (such as fluoro, for example to form 1,1-difluorocyclohex-4-yl)) or C-linked nitrogen containing heterocyclyl (such as tetrahydropyran or piperidine, optionally substituted on the ring nitrogen).

In another aspect the present invention provides a compound of the invention wherein R¹⁴ is C₁₋₈ alkyl (optionally substituted by halo (such as fluoro, for example to form CF₃CH₂)), phenyl (optionally substituted by halo) or C₅₋₆ cycloalkyl (optionally substituted by halo (such as fluoro, for example to form 1,1-difluorocyclohex-4-yl)).

In a further aspect of the invention heterocyclyl is optionally substituted (such as singly substituted for example on a ring nitrogen atom when present) by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio or S(O)₂(C₁₋₄ alkyl)}, S(O)₂NR⁴⁰R⁴¹, C(O)R⁴², C(O)NHR⁴³ or S(O)₂R⁴⁴; wherein R⁴⁰, R⁴¹, R⁴², R⁴³ and R⁴⁴ are, independently, hydrogen or C₁₋₆ alkyl.

In yet another aspect of the invention R¹ is optionally substituted aryl (such as optionally substituted phenyl) or optionally substituted heteroaryl, wherein the optional substituents are as recited above.

In a further aspect of the invention when R¹ is heterocyclyl it is, for example, tetrahydropyran, tetrahydrothiopyran, piperidine, piperazine, pyrrolidine or azetidine. In another aspect when R¹ is heterocyclyl it is, for example, piperidine, piperazine, pyrrolidine or azetidine.

In a further aspect of the invention R¹ is optionally substituted heterocyclyl, such as optionally substituted: piperidin-1-yl, piperidin-4-yl, piperazin-1-yl, pyrrolidin-1-yl, pyrrolidin-3-yl, azetidin-1-yl or azetidin-3-yl.

In a still further aspect of the invention the heterocyclyl of R¹ is mono-substituted by C₁₋₆ alkyl, C₃₋₇ cycloalkyl, phenyl {optionally substituted by halo (for example fluoro), C₁₋₄ alkyl (for example methyl), C₁₋₄ alkoxy (for example methoxy), CF₃ or OCF₃}, S(O)₂(C₁₋₄ alkyl) (for example S(O)₂CH₃, S(O)₂CH₂CH₃ or S(O)₂CH(CH₃)₂), S(O)₂(C₁₋₄ fluoroalkyl) (for example S(O)₂CF₃ or S(O)₂CH₂CF₃), S(O)₂phenyl {optionally substituted (such as mono-substituted) by halo (for example chloro), cyano, C₁₋₄ alkyl, C₁₋₄ alkoxy, CF₃, OCF₃, S(O)₂(C₁₋₄ alkyl) (for example S(O)₂CH₃ or S(O)₂CH₂CH₂CH₃) or S(O)₂(C₁₋₄ fluoroalkyl) (for example S(O)₂CH₂CF₃)}, benzyl {optionally substituted by halo (for example chloro or fluoro), C₁₋₄ alkyl, C₁₋₄ alkoxy (for example methoxy), CF₃ or OCF₃}, C(O)H, C(O)(C₁₋₄ alkyl), benzoyl {optionally substituted by halo (for example chloro or fluoro), C₁₋₄ alkyl (for example methyl), C₁₋₄ alkoxy, CF₃ or OCF₃}, C(O)₂(C₁₋₄ alkyl), C(O)NH₂, C(O)NH(C₁₋₄ alkyl) or C(O)NHphenyl {optionally substituted by halo (for example fluoro), C₁₋₄ alkyl, C₁₋₄ alkoxy, CF₃ or OCF₃}. Said heterocyclyl can also be mono-substituted by S(O)₂N(C₁₋₄ alkyl)₂. In a still further aspect when said heterocyclyl is a 4-substituted piperidin-1-yl, a 1-substituted piperidin-4-yl, a 4-substituted piperazin-1-yl, a 3-substituted pyrrolidin-1-yl, a 1-substituted pyrrolidin-3-yl, a 3-substituted azetidin-1-yl or a 1-substituted azetidin-3-yl (for example where said substituent is as recited earlier in this paragraph). In another aspect said heterocyclyl is a 1-substituted piperidin-4-yl or a 4-substituted piperazin-1-yl, wherein the substituent is S(O)₂(C₁₋₄ alkyl), S(O)₂(C₁₋₄ haloalkyl), S(O)₂(phenyl), S(O)₂N(C₁₋₄ alkyl)₂ or phenyl.

In another aspect of the invention R¹ is piperidinyl or piperazinyl (such as piperidin-4-yl or piperazin-1-yl), either of which is N-substituted by phenyl, S(O)₂R³⁹ (wherein R³⁹ is C₁₋₄ alkyl (such as methyl or ethyl), phenyl or CF₃) or S(O)₂NR²⁹R³⁰ (wherein R²⁹ and R³⁰ are, independently, C₁₋₄ alkyl (such as methyl)).

In yet another aspect of the invention R¹ is NHC(O)R¹⁴ wherein R1⁴ is C₁₋₄ haloalkyl (for example C₁₋₄ fluoroalkyl, such as CH₂CF₃ or CH₂CH₂CF₃), phenyl (optionally substituted by halo) or C₃₋₆ cycloalkyl (substituted by one or two fluoros).

In a further aspect of the invention R¹ is phenyl optionally substituted by S(O)₂R³⁹ (wherein R³⁹ is C₁₋₄ alkyl (such as methyl)).

In a still further aspect of the invention R¹ is heteroaryl (such as pyridinyl) optionally substituted by CF₃.

In another aspect of the invention R¹ is heterocyclyl (such as tetrahydropyran or tetrahydrothiopyran).

In yet another aspect of the invention R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃; wherein n is 0, 1 or 2, for example 0 or 2. When R² is heteroaryl it is, for example an optionally substituted thiophenyl (that is, thienyl).

In another aspect R² is phenyl or thienyl, either of which is optionally substituted by halo (such as chloro or fluoro) or CF₃.

In a still further aspect R² is optionally substituted (for example unsubstituted or substituted in the 2-, 3-, or 3- and 5-positions) phenyl (such as optionally substituted by halo (such as chloro or fluoro), cyano, methyl, ethyl, methoxy, ethoxy or CF₃), or optionally substituted (for example unsubstituted or mono-substituted) heteroaryl (such as optionally substituted by halo (such as chloro or fluoro), cyano, methyl, ethyl, methoxy, ethoxy or CF₃).

In another aspect the invention provides a compound of the invention wherein R² is optionally substituted (for example unsubstituted or substituted in the 2-, 3-, or 3- and 5-positions) phenyl (such as optionally substituted by halo (for example chloro or fluoro)). In yet another aspect the invention provides a compound of the invention wherein R² is phenyl, 3-fluorophenyl, 3-chlorophenyl, 3-trifluoromethylphenyl, 3-chloro-5-fluorophenyl or 3,5-difluorophenyl. In a further aspect the invention provides a compound of the invention wherein R² is phenyl, 3-fluorophenyl, 3-chlorophenyl or 3,5-difluorophenyl.

In yet another aspect of the invention R³ is hydrogen or methyl. In a further aspect of the invention when R³ is C₁₋₄ alkyl (such as methyl) and the carbon to which R³ is attached has the R absolute configuration. In yet another aspect of the invention R³ is hydrogen.

In a still further aspect the present invention provides a compound of the invention wherein R⁴ is optionally substituted phenyl (the optional substituents being selected from those recited above).

In another aspect the present invention provides a compound of the invention wherein R⁴ is optionally substituted aryl (such as phenyl) or optionally substituted heteroaryl (such as pyridyl, imidazolyl or 1,3,4-thiadiazolyl), (the optional substituents being selected from those recited above).

In yet another aspect the present invention provides a compound of the invention wherein R⁴ is phenyl optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl groups (such as tetrazolyl) are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃ }and in a further aspect of the invention the foregoing heteroaryl groups (such as tetrazolyl) are optionally substituted by C₁₋₄ alkyl}.

In a further aspect the present invention provides a compound of the invention wherein R⁴ is phenyl optionally substituted by halogen (such as chloro or fluoro), cyano, C₁₋₄ alkyl (mono-substituted by S(O)₂(C₁₋₄ alkyl) or C(O)NH(C₁₋₄ alkyl), C₁₋₄ alkoxy, S(C₁₋₄ alkyl), S(O)₂(C₁₋₄ alkyl), OS(O)₂(C₁₋₄ alkyl), OCH₂COOH, OCH₂-tetrazolyl (itself optionally substituted by C₁₋₄ alkyl), carboxamide or tetrazolyl (itself optionally substituted by C₁₋₄ alkyl).

In yet another aspect the present invention provides a compound of the invention wherein R⁴ is aryl or heteroaryl each being optionally substituted by OS(O)₂R⁴⁹ or C₁₋₆ alkyl (mono-substituted by S(O)₂R⁵⁰ or C(O)NR⁵¹R⁵²); wherein R⁴⁹, R⁵⁰, R⁵¹ and R⁵² are above.

In a further aspect the present invention provides a compound of the invention wherein R⁴ is phenyl (optionally substituted by halogen (such as chloro or fluoro), cyano, C₁₋₄ alkyl, C₁₋₄ alkoxy, S(C₁₋₄ alkyl), S(O)₂(C₁₋₄ alkyl), OS(O)₂(C₁₋₄ alkyl) or carboxamide), C₃₋₇ cycloalkyl (such as cyclohexyl), pyridyl (optionally substituted by C₁₋₄ alkyl), imidazolyl (optionally substituted by C₁₋₄ alkyl) or 1,3,4-thiadiazolyl (optionally substituted by C₁₋₄ alkyl).

In a further aspect the present invention provides a compound of the invention wherein R⁴ is phenyl {optionally substituted by S(O)₂(C₁₋₄ alkyl) (such as CH₃S(O)₂, for example in the 4-position), C₁₋₄ alkoxy (such as CH₃O, for example in the 4-position), OS(O)₂(C₁₋₄ alkyl) (such as OSO₂CH₃, for example in the 4-position), halogen (such as chloro or fluoro) or cyano}.

In a still further aspect the invention provides a compound of the invention wherein A is absent.

In another aspect the invention provides a compound of the invention wherein X is O or S(O)₂. In yet another aspect X is S(O)₂.

In a further aspect the invention provides a compound of the invention wherein m is 2 and n is0or n is 2 and m is 0.

In a still further aspect the invention provides a compound of the invention wherein p is 0.

In another aspect the invention provides a compound of the invention wherein X is O and m and n are not both 1.

In yet another aspect the invention provides a compound of the invention wherein X is S(O)₂ and m and n are both 1.

In a further aspect the invention provides a compound of the invention wherein X is S(O)₂, n is 2 and m is 0.

In a still further aspect the invention provides a compound of the invention wherein X is S(O)₂, n is 0 and m is 2.

In another aspect the invention provides a compound of the invention wherein X is O and m and n are both 1.

In a still further aspect the present invention provides a compound of formula (Ia): 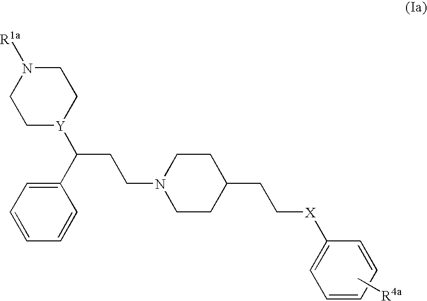
wherein X is as defined above; Y is CH or N; R^{4a} is as defined for optional substituents on optionally substituted phenyl (above); and R^{1a} is mono-substituted by C₁₋₆ alkyl, C₃₋₇ cycloalkyl, phenyl {optionally substituted by halo (for example fluoro), C₁₋₄ alkyl (for example methyl), C₁₋₄ alkoxy (for example methoxy), CF₃ or OCF₃}, S(O)₂(C₁₋₄ alkyl) (for example S(O)₂CH₃, S(O)₂CH₂CH₃ or S(O)₂CH(CH₃)₂), S(O)₂(C₁₋₄ fluoroalkyl) (for example S(O)₂CF₃ or S(O)₂CH₂CF₃), S(O)₂phenyl {optionally substituted (such as mono-substituted) by halo (for example chloro), cyano, C₁₋₄ alkyl, C₁₋₄ alkoxy, CF₃, OCF₃, S(O)₂(C₁₋₄ alkyl) (for example S(O)₂CH₃ or S(O)₂CH₂CH₂CH₃) or S(O)₂(C₁₋₄ fluoroalkyl) (for example S(O)₂CH₂CF₃)}, benzyl {optionally substituted by halo (for example chloro or fluoro), C₁₋₄ alkyl, C₁₋₄ alkoxy (for example methoxy), CF₃ or OCF₃}, C(O)H, C(O)(C₁₋₄ alkyl), benzoyl {optionally substituted by halo (for example chloro or fluoro), C₁₋₄ alkyl (for example methyl), C₁₋₄ alkoxy, CF₃ or OCF₃}, C(O)₂(C₁₋₄ alkyl), C(O)NH₂, C(O)NH(C₁₋₄ alkyl) or C(O)NHphenyl {optionally substituted by halo (for example fluoro), C₁₋₄ alkyl, C₁₋₄ alkoxy, CF₃ or OCF₃}. R^{1a} can also be S(O)₂N(C₁₋₄ alkyl)₂.

In another aspect the present invention provides a compound of formula (Ib): 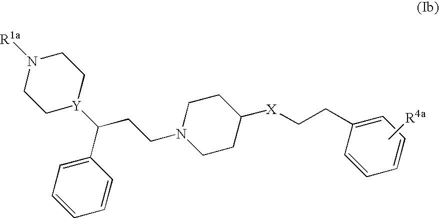
wherein X, Y, R^{1a} and R^{4a} are as defined above.

In yet another aspect the present invention provides a compound of formula (Ic): 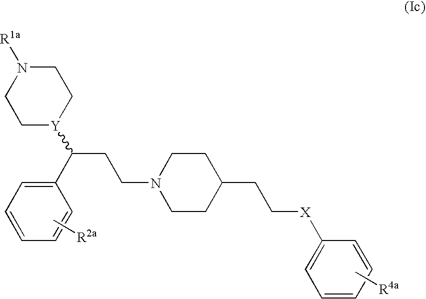
wherein X, Y, R^{1a} and R^{4a} are as defined above, and R^{2a} is hydrogen, one or two halogen atoms (for example selected from chlorine and fluorine) or CF₃. In another aspect of the invention R^{2a} is hydrogen.

In a further aspect the present invention provides a compound of formula (Id): 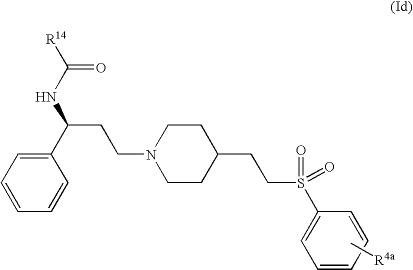
wherein R¹⁴ and R^{4a} are as defined above.

In a still further aspect the present invention provides a compound of formula (Ie): 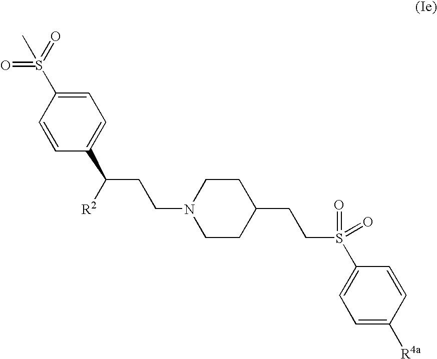
wherein R² and R^{4a} are as defined above.

In another aspect the present invention provides a compound of formula (If): 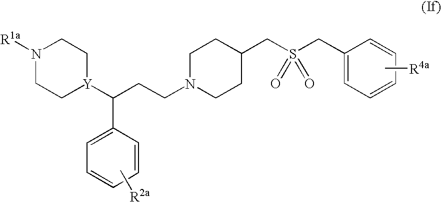
wherein Y, R^{1a}, R^{2a} and R^{4a} are as defined above.

In yet another aspect the present invention provides a compound of formula (Ig): 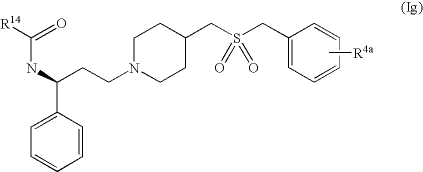
wherein R¹⁴ and R^{4a} are as defined above.

In a further aspect the present invention provides a compound of formula (Ih): 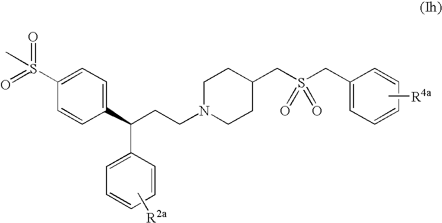
wherein R^{2a} and R^{4a} are as defined above.

In a still further aspect the present invention provides a compound of formula (Ii): 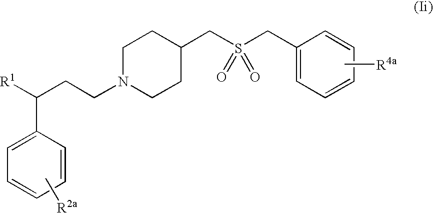
wherein R¹, R^{2a} and R^{4a} are as defined above.

In another aspect the present invention provides a compound of formula (Ij): 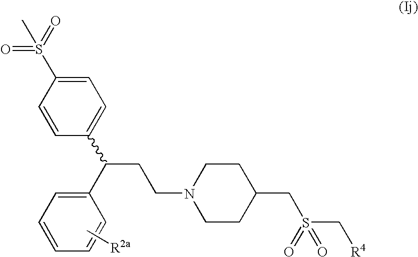
wherein R^{2a} and R⁴ are as defined above.

In yet another aspect the present invention provides a compound of formula (Ik): 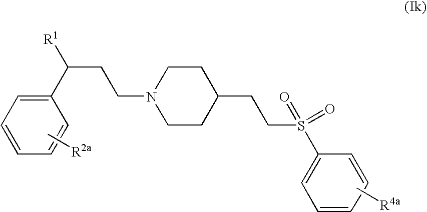
wherein R¹, R^{2a} and R^{4a} are as defined above.

In a further aspect the present invention provides a compound of formula (Il): 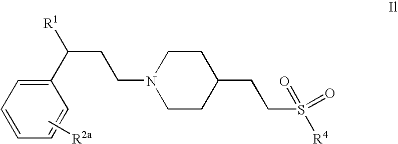
wherein R¹, R^{2a} and R⁴ are as defined above.

In a still further aspect the present invention provides a compound of formula (Im): 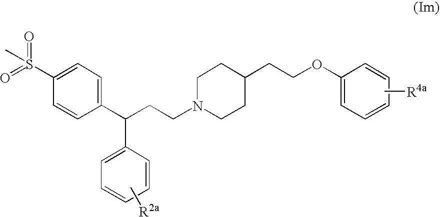
wherein R^{2a} and R^{4a} are as defined above.

In another aspect the present invention provides a compound of formula (In): 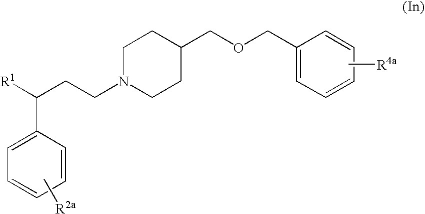
wherein R¹, R^{2a} and R^{4a} are as defined above.

In yet another aspect of the invention there is provided a compound of formula (Ia), (Ib), (Ic) or (If) wherein R^{1a} is S(O)₂(C₁₋₄ alkyl), S(O)₂(C₁₋₄ haloalkyl), S(O)₂(phenyl), S(O)₂N(C₁₋₄ alkyl)₂ or phenyl.

In yet another aspect of the invention there is provided a compound of formula (Ic), (If), (Ih), (Ii), (Ij), (Ik), (Im) or (In) wherein R^{2a} is hydrogen, one or two halo (such as one chloro, one fluoro, one chloro and one fluoro or two fluoro) or CF₃. R^{2a} is, for example in the 2-, 3-, or 3- and 5-positions on the phenyl ring.

In another aspect of the invention there is provided a compound of formula (Ia), (Ib), (Ic), (Id), (If), (Ig), (Ih), (Ii), (Ik), (Im) or (In) wherein R^{4a} is in the 4-position on the phenyl ring.

In a further aspect of the invention there is provided a compound of formula (Ia), (Ib), (Ic), (Id), (Ie), (If), (Ig), (Ih), (Ii), (Ik), (Im) or (In) wherein R^{4a} is one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl (such as OCH₂tetrazolyl), OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl group (such as tetrazolyl) are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃ {and in a further aspect of the invention the foregoing heteroaryl groups (such as tetrazolyl) are optionally substituted by C₁₋₄ alkyl}.

In a still further aspect of the invention there is provided a compound of formula (Ia), (Ib), (Ic), (Id), (Ie), (If), (Ig), (Ih), (Ii), (Ij), (Ik), (Im) or (In) wherein R^{4a} is halogen (such as chloro or fluoro), cyano, C₁₋₄ alkyl, C₁₋₄ alkoxy, S(C₁₋₄ alkyl), S(O)₂(C₁₋₄ alkyl), OS(O)₂(C₁₋₄ alkyl) or carboxamide.

The compounds listed in Tables I to XIV illustrate the invention.

TABLE I
Table I comprises compounds of formula (Ia)

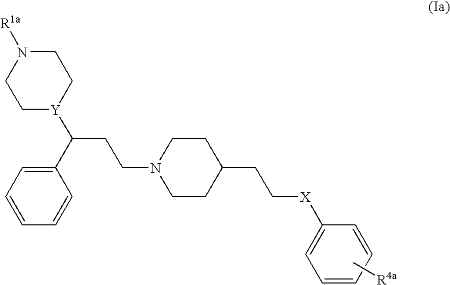

| Compound No | Y | R^{1a} | X | R^{4a} | MS (MH+) |
|---|---|---|---|---|---|
| 1 | CH | ethanesulphonyl | O | H | 499 |
| 2 | N | benzenesulphonyl | O | H | 548 |
| 3 | N | benzenesulphonyl | O | 4-methanesulphonyl | 626 |
| 4 | N | ethanesulphonyl | O | 4-methanesulphonyl | 578 |
| 5 | N | benzenesulphonyl | S(O)₂ | 4-methanesulphonyl | 674 |
| 6 | N | methanesulphonyl | S | 4-methylthio | 562 |
| 7 | N | ethanesulphonyl | S | 4-methylthio | 548 |
| 8 | N | phenyl | S(O)₂ | 4-methanesulphonyl | 610 |
| 9 | N | methanesulphonyl | S(O)₂ | 4-methanesulphonyl | 612 |
| 10 | N | ethanesulphonyl | S(O)₂ | 4-methanesulphonyl | 626 |
| 11 | CH | methanesulphonyl | S(O)₂ | 4-fluoro | 551 |
| 12 | N | phenyl | S(O)₂ | 4-fluoro | 550 |
| 13 | CH | methanesulphonyl | S(O)₂ | 4-methanesulphonyl | 611 |
| 14 | CH | methanesulphonyl | S(O)₂ | 4-chloro | 567 |
| 15 | CH | trifluoromethanesulphonyl | S(O)₂ | 4-chloro | 621 |
| 16 | CH | methanesulphonyl | S(O)₂ | Hydrogen | 533 |
| 17 | CH | methanesulphonyl | S(O)₂ | 4-methyl | 547 |
| 18 | CH | methanesulphonyl | S(O)₂ | 4-trifluoromethyl | 601 |
| 19 | CH | methanesulphonyl | S(O)₂ | 4-methoxy | 563 |
| 20 | CH | methanesulphonyl | S(O)₂ | 4-cyano | 558 |

TABLE II
Table II comprises compounds of formula (Ib)

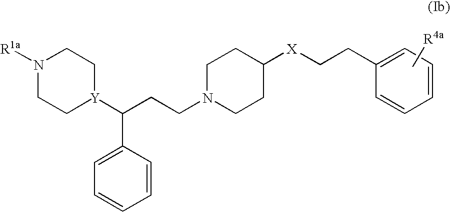

| Compound No | R^{1a} | Y | X | m | R^{4a} | MS (MH+) |
|---|---|---|---|---|---|---|
| 1 | benzenesulphonyl | N | S(O)₂ | 2 | 4-methanesulphonyl | 674 |
| 2 | phenyl | N | S(O)₂ | 2 | 4-methanesulphonyl | 610 |

TABLE III
Table III comprises compounds of formula (Ic)

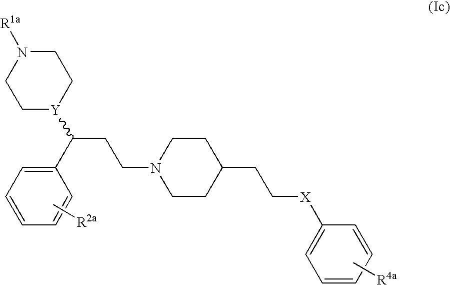

| Compound No | R^{1a} | Y | Stereochemistry | R^{2a} | X | R^{4a} | MS (MH+) |
|---|---|---|---|---|---|---|---|
| 1 | phenyl | N | R or S | H | S(O)₂ | 4-methanesulphonyl | 610 |
| 2 | phenyl | N | S or R | H | S(O)₂ | 4-methanesulphonyl | 610 |
| 3 | methanesulphonyl | CH | R or S | H | S(O)₂ | 4-fluoro | 551 |
| 4 | methanesulphonyl | CH | S or R | H | S(O)₂ | 4-fluoro | 551 |
| 5 | methanesulphonyl | N | S or R | H | S(O)₂ | hydrogen | 534 |
| 6 | benzenesulphonyl | N | S or R | H | S(O)₂ | hydrogen | 596 |
| 7 | methanesulphonyl | N | S or R | H | S(O)₂ | 4-methoxy | 564 |
| 8 | trifluoromethane-sulphonyl | N | S or R | H | S(O)₂ | 4-methoxy | 618 |
| 9 | methanesulphonyl | N | S or R | H | S(O)₂ | 4-trifluoromethyl | 602 |
| 10 | methanesulphonyl | N | S or R | H | S(O)₂ | 4-methyl | 548 |
| 11 | trifluoromethane-sulphonyl | N | S or R | H | S(O)₂ | 4-methyl | 602 |
| 12 | trifluoromethane-sulphonyl | N | S or R | H | S(O)₂ | 4-trifluoromethyl | 656 |
| 13 | trifluoromethane-sulphonyl | N | S or R | H | S(O)₂ | hydrogen | 588 |
| 14 | methanesulphonyl | N | S or R | H | S(O)₂ | 4-fluoro | 552 |
| 15 | methanesulphonyl | N | S or R | H | S(O)₂ | 4-chloro | 568 |
| 16 | benzenesulphonyl | N | S or R | H | S(O)₂ | 4-trifluoromethyl | 664 |
| 17 | trifluoromethane-sulphonyl | N | S or R | H | S(O)₂ | 4-fluoro | 606 |
| 18 | trifluoromethane-sulphonyl | N | S or R | H | S(O)₂ | 4-chloro | 622 |
| 19 | methanesulphonyl | N | S or R | H | S(O)₂ | 4-methanesulphonyl | 612 |
| 20 | trifluoromethane-sulphonyl | N | S or R | H | S(O)₂ | 4-methanesulphonyl | 666 |
| 21 | dimethylaminosulphonyl | CH | R or S | H | S(O)₂ | 4-methanesulphonyl | 640 |
| 22 | methanesulphonyl | CH | R or S | H | S(O)₂ | 4-methanesulphonyl | 611 |
| 23 | methanesulphonyl | CH | R or S | H | S(O)₂ | 4-methoxy | 563 |
| 24 | methanesulphonyl | CH | R or S | H | S(O)₂ | 4-methylenecarboxamide | 590 |
| 25 | methanesulphonyl | CH | R or S | H | S(O)₂ | 4-methanesulphonyl-methyl | 625 |
| 26 | methanesulphonyl | CH | R or S | H | S(O)₂ | 4-carboxamide | 576 |
| 27 | methanesulphonyl | CH | R or S | H | S(O)₂ | 4-cyano | 558 |
| 28 | methanesulphonyl | CH | R or S | H | S(O)₂ | 4-hydroxy | 549 |
| 29 | methanesulphonyl | CH | R or S | H | S(O)₂ | 4-methanesulphonyloxy | 627 |
| 30 | methanesulphonyl | CH | R or S | H | S(O)₂ | 4-(tetrazol-5-yl) | 601 |
| 31 | dimethylaminosulphonyl | CH | R | 3,5-difluoro | S(O)₂ | 4-methanesulphonyl | 676 |
| 32 | methanesulphonyl | CH | R | 3,5-difluoro | S(O)₂ | 4-methanesulphonyl | 647 |
| 33 | methanesulphonyl | CH | R | 3-trifluoromethyl | S(O)₂ | 4-methanesulphonyl | 679 |
| 34 | methanesulphonyl | CH | R | 3,5-difluoro | S(O)₂ | 4-methoxy | 599 |
| 35 | methanesulphonyl | CH | R | H | S(O)₂ | 2-methyl-tetrazol-5-yl | 615 |
| 36 | methanesulphonyl | CH | R | H | S(O)₂ | 4-[(2-methyl-tetrazol-5- | 645 |
| | | | | | | yl)methyleneoxy] | |
| 37 | methanesulphonyl | CH | R | H | S(O)₂ | 4-[(1-methyl-tetrazol-5- | 645 |
| | | | | | | yl)methyleneoxy] | |

TABLE IV
Table IV comprises compounds of formula (Id)

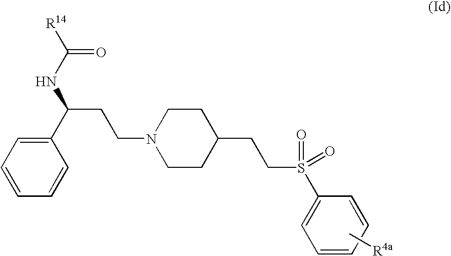

| Compound No | R¹⁴ | Stereochemistry | R^{4a} | MS (MH+) |
|---|---|---|---|---|
| 1 | 2,2,2-trifluoroethyl | S | 4-methanesulphonyl | 575 |
| 2 | 4-chlorophenyl | S | 4-methanesulphonyl | 603 |
| 3 | 2,2,2-trifluoroethyl | S | 4-methyl | 511 |
| 4 | 2,2,2-trifluoroethyl | S | 4-fluoro | 515 |
| 5 | 3,3,3-trifluoropropyl | S | 4-methanesulphonyl | 589 |
| 6 | 3,3-difluorocyclobutyl | S | 4-methanesulphonyl | 583 |
| 7 | 4,4-difluorocyclohexyl | S | 4-methanesulphonyl | 611 |

TABLE V
Table V comprises compounds of formula (Ie)

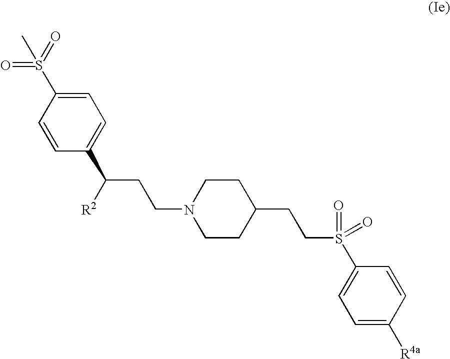

| Compound No. | R² | R^{4a} | MS (MH+) |
|---|---|---|---|
| 1 | 2-thienyl | methanesulphonyl | 610 |
| 2 | 3-thienyl | methanesulphonyl | 610 |
| 3 | phenyl | methanesulphonyl | 604 |
| 4 | phenyl | fluoro | 544 |
| 5 | 5-chloro-2-thienyl | methanesulphonyl | 645 |
| 6 | 4-chloro-2-thienyl | methanesulphonyl | 645 |
| 7 | 3,5-difluorophenyl | methanesulphonyl | 640 |
| 8 | 3,5-difluorophenyl | fluoro | 580 |
| 9 | 3,5-difluorophenyl | hydrogen | 562 |
| 10 | 3,5-difluorophenyl | methoxy | 592 |
| 11 | 3,5-difluorophenyl | nitro | 607 |
| 12 | 3,5-difluorophenyl | trifluoromethoxy | 646 |
| 13 | 3,5-difluorophenyl | acetylamino | 619 |
| 14 | 3,5-difluorophenyl | amino | 577 |
| 15 | 3,5-difluorophenyl | cyanomethyleneoxy | 617 |
| 16 | 3,5-difluorophenyl | oxyacetamide | 635 |
| 17 | 3,5-difluorophenyl | (1H-tetrazol-5-yl)methoxy | 660 |
| 18 | 3,5-difluorophenyl | methanesulphonylamino | 655 |
| 19 | 3,5-difluorophenyl | 2-methyl-tetrazol-5-yl | 644 |
| 20 | 3,5-difluorophenyl | 1-methyl-tetrazol-5-yl | 644 |
| 21 | 3,5-difluorophenyl | phenylaminocarbonylamino | 696 |
| 22 | 3,5-difluorophenyl | hydroxy | 578 |
| 23 | 3,5-difluorophenyl | methanesulphonyloxy | 656 |
| 24 | 3,5-difluorophenyl | (4-toluene)sulphonyloxy | 732 |
| 25 | 3,5-difluorophenyl | [(2-methyl-tetrazol-5-yl)methyleneoxy] | 674 |
| 26 | 3,5-difluorophenyl | [(1-methyl-tetrazol-5-yl)methyleneoxy] | 674 |
| 27 | 3,5-difluorophenyl | methylcarboxymethoxy | 650 |
| 28 | 3,5-difluorophenyl | carboxymethoxy | 636 |
| 29 | 3,5-difluorophenyl | (N-[methanesulphonyl]carbamoylmethoxy) | 713 |
| 30 | 3,5-difluorophenyl | N-methylcarbamoylmethoxy | 649 |

TABLE VI
Table VI comprises compounds of formula (If):

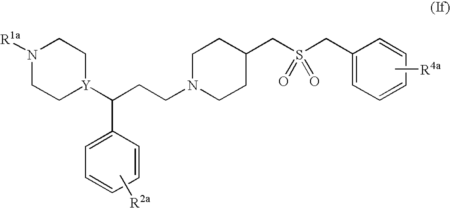

| Compound No | Y | Stereochemistry | R^{1a} | R^{2a} | R^{4a} | MS (MH+) |
|---|---|---|---|---|---|---|
| 1 | CH | R | methanesulphonyl | H | H | 533 |
| 2 | CH | R | methanesulphonyl | H | 4-methoxy | 563 |
| 3 | CH | R | methanesulphonyl | H | 4-methyl | 547 |
| 4 | CH | R | methanesulphonyl | H | 4-fluoro | 551 |
| 5 | CH | R | methanesulphonyl | H | 4-methanesulphonyl | 611 |
| 6 | CH | R | methanesulphonyl | 3,5-difluoro | 4-methanesulphonyl | 647 |
| 7 | N | S | methanesulphonyl | H | 4-methanesulphonyl | 612 |
| 8 | N | S | trifluoromethanesulphonyl | H | 4-methanesulphonyl | 666 |
| 9 | CH | R | methanesulphonyl | H | 4-cyano | 558 |
| 10 | CH | R | methanesulphonyl | H | 4-carboxamide | 576 |

TABLE VII
Table VII comprises compounds of formula (Ig):

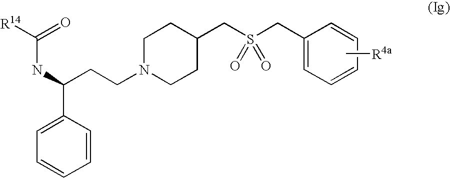

| Compound No | R¹⁴ | Stereochemistry | R^{4a} | MS (MH+) |
|---|---|---|---|---|
| 1 | 4,4-difluorocyclohexyl | S | 4-methanesulphonyl | 611 |

TABLE VIII
Table VIII comprises compounds of formula (Ih):

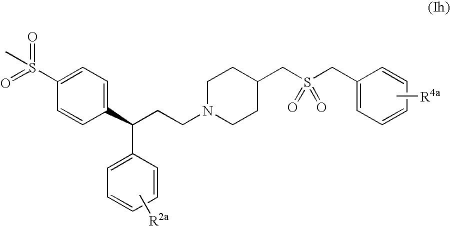

| Compound No | Stereochemistry | R^{2a} | R^{4a} | MS (MH+) |
|---|---|---|---|---|
| 1 | S | H | H | 526 |
| 2 | R | 3,5-difluoro | 4-methanesulphonyl | 640 |
| 3 | R | 3,5-difluoro | 4-methoxy | 592 |
| 4 | R | 3,5-difluoro | 4-cyano | 587 |
| 5 | R | 3,5-difluoro | 4-carboxamide | 605 |

TABLE IX
Table IX comprises compounds of formula (Ii):

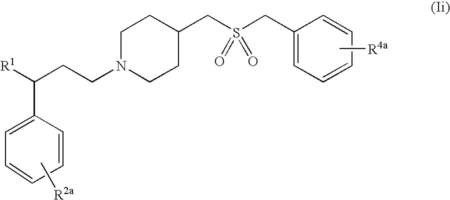

| Compound No | R¹ | R^{2a} | Stereochemistry | R^{4a} | MS (MH+) |
|---|---|---|---|---|---|
| 1 | 6-trifluoromethylpyridin-3-yl | 3,5-difluoro | S | 4-methanesulphonyl | 631 |

TABLE X
Table X comprises compounds of formula (Ij):

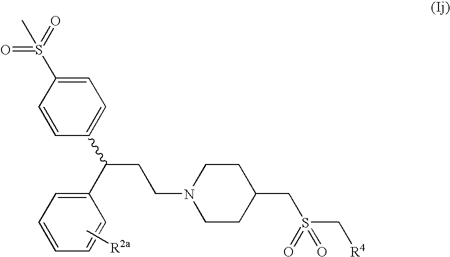

| Compound No | Stereochemistry | R^{2a} | R⁴ | MS (MH+) |
|---|---|---|---|---|
| 1 | R | 3,5-difluoro | 6-trifluoromethylpyridin-3-yl | 631 |
| 2 | R | 3,5-difluoro | pyridin-2-yl | 563 |
| 3 | R | 3,5-difluoro | pyridin-4-yl | 563 |

TABLE XI
Table XI comprises compounds of formula (Ik):

| | |
|---|---|
| | (Ik) |
| 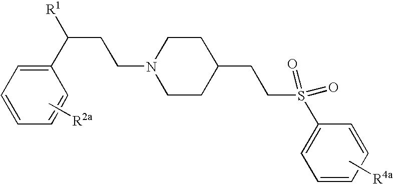 | |

| Compound No | R¹ | R^{2a} | Stereochemistry | R^{4a} | MS (MH+) |
|---|---|---|---|---|---|
| 1 | 4-methanesulphonylphenyl | 3,5-difluoro | R | 4-methanesulphonylmethyl | 654 |
| 2 | 4-methanesulphonylphenyl | 3,5-difluoro | R | 3-fluoro | 580 |
| 3 | 6-trifluoromethylpyridin-3-yl | 3,5-difluoro | S | 4-methanesulphonyl | 631 |
| 4 | 4-methanesulphonylphenyl | 3-chloro-5-fluoro | R | 4-methanesulphonyl | 656 |
| 5 | 4-methanesulphonylphenyl | 3,5-difluoro | R | 3-chloro | 596 |
| 6 | 4-methanesulphonylphenyl | 3,5-difluoro | R | 3-trifluoromethyl | 630 |
| 7 | 4-methanesulphonylphenyl | 3,5-difluoro | R | 2,4-difluoro | 598 |
| 8 | 4-methanesulphonylphenyl | 3,5-difluoro | R | 3,4-difluoro | 598 |
| 9 | 3-methanesulphonylphenyl | hydrogen | RS | 4-methanesulphonyl | 604 |
| 10 | 4-methanesulphonylphenyl | 3,5-difluoro | R | 4-cyanomethyleneoxy | 617 |
| 11 | 4-methanesulphonylphenyl | 3,5-difluoro | R | 4-oxyacetamide | 635 |
| 12 | 4-methanesulphonylphenyl | 3,5-difluoro | R | 3-hydroxy | 578 |
| 13 | 4-methanesulphonylphenyl | 3,5-difluoro | R | 3-methanesulphonyloxy | 656 |
| 14 | 4-methanesulphonylphenyl | 3,5-difluoro | R | 3-(4-toluene)sulphonyloxy | 732 |
| 15 | 4-methanesulphonylphenyl | 3,5-difluoro | R | 3,4-dimethoxy | 622 |
| 16 | 4-methanesulphonylphenyl | 3-chloro-5-fluoro | R | 4-methanesulphonyl | 656 |
| 17 | 4-tetrahydropyranyl | 3,5-difluoro | R | 4-methanesulphonyl | 570 |
| 18 | 4-tetrahydrothiopyranyl | 3,5-difluoro | R | 4-methanesulphonyl | 586 |
| 19 | 4-tetrahydropyranyl | hydrogen | R | 4-methanesulphonyl | 534 |
| 20 | 4-methanesulphonylphenyl | 3,5-difluoro | R | 3-chloro-4-fluoro | 614 |

TABLE XII
Table XII comprises compounds of formula (II):

| | |
|---|---|
| | (II) |
| 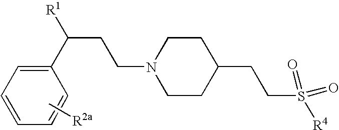 | |

| Compound No | R¹ | R^{2a} | R⁴ | MS (MH+) |
|---|---|---|---|---|
| 1 | (R) N-MeS(O)₂-piperidin-4-yl | hydrogen | cyclohexyl | 539 |
| 2 | (R) N-MeS(O)₂-piperidin-4-yl | hydrogen | methyl | 471 |
| 3 | (R) N-MeS(O)₂-piperidin-4-yl | hydrogen | 3-pyridyl | 534 |
| 4 | (R) N-MeS(O)₂-piperidin-4-yl | hydrogen | 5-methyl-1,3,4-thiadiazol-2-yl | 555 |
| 5 | (R) N-MeS(O)₂-piperidin-4-yl | hydrogen | 1-methyl-imidazol-2-yl | 537 |
| 6 | (R) 4-MeS(O)₂-phenyl | 3,5-difluoro | 3-pyridyl | 563 |
| 7 | (R) 4-MeS(O)₂-phenyl | 3,5-difluoro | 5-methyl-1,3,4-thiadiazol-2-yl | 584 |
| 8 | (R) 4-MeS(O)₂-phenyl | 3,5-difluoro | 1-methyl-imidazol-2-yl | 566 |
| 9 | (R) 4-MeS(O)₂-phenyl | 3,5-difluoro | 6-methylpyridin-3-yl | 577 |

TABLE XIII
Table XIII comprises compounds of formula (Im):

| | |
|---|---|
| | (Im) |
| 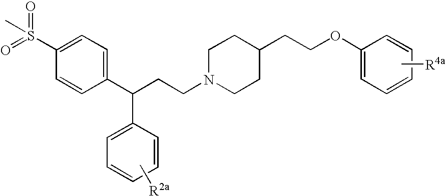 | |

| Compound | | Stereo- | | |
|---|---|---|---|---|
| No | R^{2a} | chemistry | R^{4a} | MS (MH+) |
| 1 | 3,5-di- | R | 4-methanesulphonyl | 592 |
| | fluoro | | | |

TABLE XIV
Table XIV comprises compounds of formula (In):

| | |
|---|---|
| | (In) |
| 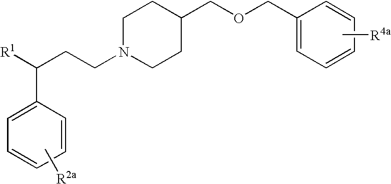 | |

| Compound No | R¹ | R^{2a} | Stereochemistry | R^{4a} | MS (MH+) |
|---|---|---|---|---|---|
| 1 | 4-methanesulphonylphenyl | 3,5-difluoro | R | 4-methanesulphonyl | 592 |
| 2 | 4-methanesulphonylpiperidin-4-yl | hydrogen | R | 4-methanesulphonyl | 563 |

In yet another aspect the invention provides each individual compound listed in the tables above.

The compounds of formula (I), (Ia), (Ib), (Ic), (Id), (Ie), (If), (Ig), (Ih), (Ii), (Ij), (Ik), (Il), (Im) and (In) are all compounds of the invention can be prepared as shown below.

A compound of the invention wherein R¹ is an N-linked optionally substituted heterocycle can be prepared by reacting a compound of formula (II): 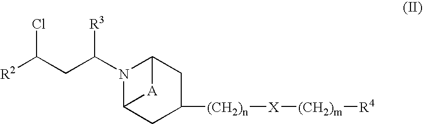
wherein R², R³, R⁴, m, n, A and X are as defined above, with a compound R¹H (wherein the H is on a heterocycle ring nitrogen atom) wherein R¹ is as defined above, in the presence of a suitable base (for example a tri(C₁₋₆ alkyl)amine such as triethylamine or Hunig's base), in a suitable solvent (such as a chlorinated solvent, for example dichloromethane) and, for example, at a room temperature (for example 10-30° C.), optionally in the presence of sodium iodide.

A compound of the invention, wherein R³ is hydrogen, can be prepared by coupling a compound of formula (III): 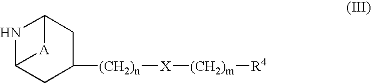
wherein R⁴, m, n, A and X are as defined above, with a compound of formula (IV): 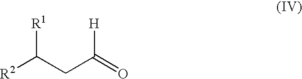
wherein R¹ and R² are as defined above, in the presence of NaBH(OAc)₃ (wherein Ac is C(O)CH₃) in a suitable solvent (such as a chlorinated solvent, for example dichloromethane) at room temperature (for example 10-30° C.).

A compound of the invention, wherein R³ is hydrogen, can be prepared by coupling a compound of formula (III): 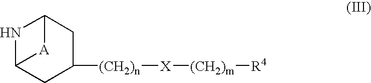
wherein R⁴, m, n, A and X are as defined above, with a compound of formula (V):

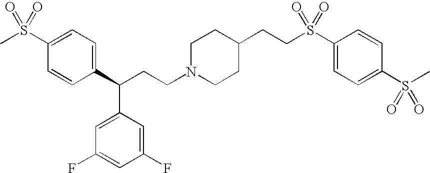

(R)-3-(3,5-Difluorophenyl)-3-(4-methanesulfonylphenyl)propionaldehyde (0.357 g, 1.1 mmol; Method E) was dissolved in dichloromethane (3 ml) at room temperature and 4-[2-(4-methanesulphonylphenyl-sulphonyl)ethyl]piperidine hydrochloride (0.368 g, 1 mmol; Method B) was added as a single portion. After stirring for 0.5 h, sodium triacetoxyborohydride (0.211 g, 1 mmol) was added as a single portion and the reaction stirred for a further 1 h. The mixture was then washed with saturated aqueous sodium hydrogen carbonate, the organics were separated and poured directly onto an SCX column. Eluting with methanol followed by 20% 7M ammonia in methanol gave the product (0.319 g, 50%) as a white solid.

NMR: (d6-DMSO): 1.05 (m, 2), 1.15 (m, 1H), 1.6 (m, 4H), 1.8 (br t, 2H), 2.2 (m, 2H), 2.3 (m, 2H), 2.8 (br d, 2H), 3.4 (m, 6H), 3.5 (m, 2H), 4.3 (br t, 1H), 7.1 (br t, 1H), 7.2 (d, 2H), 7.7 (d, 2H), 7.9 (d, 2H), 8.3 (m, 4H).

LCMS: 640.2 (ME⁺).

### EXAMPLE 5

### This Example Illustrates the Preparation of (R or S) N-(3-[4-methanesulphonylpiperazinyl]-3-phenylpropyl)-4-[2-(4-fluorophenylsulphonyl)ethyl]-piperidine (Compound 14, Table III)

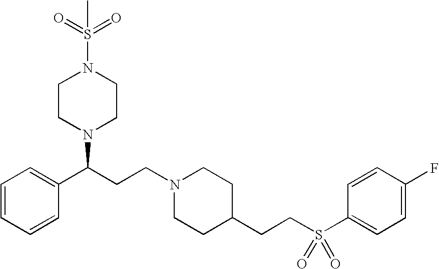

A solution of (R or S) N-(3-chloro-3-phenylpropyl)-4-[2-(4-fluorophenylsulphonyl)-ethyl]-piperidine (Method F; 310 mg) in dichloromethane (6 ml) was added to N-methanesulphonyl-piperazine hydrochloride (150 mg) followed by triethylamine (313 μl). The mixture was stirred for 48 hours, diluted with dichloromethane (5 ml) and MP-carbonate 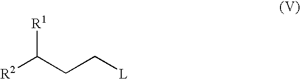
wherein R¹ and R² are as defined above and L is a leaving group such as halogen, tosylate, mesylate or triflate, in the presence of a base, such as potassium carbonate, in a suitable solvent (such as dioxane, acetonitrile or isopropanol) at temperatures from 60° C. up to the boiling point of the solvent.

Alternatively, compounds of the invention can be prepared according to Schemes 1-7 (below).

Alternatively, compounds of the invention can be prepared by using or adapting methods described in WO01/87839, EP-A1-1013276, WO00/08013, WO99/38514, WO99/04794; WO00/76511, WO00/76512, WO00/76513, WO00/76514, WO00/76972 or US 2002/0094989.

The starting materials for these processes are either commercially available or can be prepared by literature methods, adapting literature methods or by following or adapting Methods herein described.

In a still further aspect the invention provides processes for preparing the compounds of formula (I), (Ia), (Ib), (Ic), (Id), (Ie), (If), (Ig), (Ih), (Ii), (Ij), (Ik), (Il), (Im) and (In). Many of the intermediates in the processes are novel and these are provided as further features of the invention.

The compounds of the invention have activity as pharmaceuticals, in particular as modulators (such as agonists, partial agonists, inverse agonists or antagonists) of chemokine receptor (such as CCR5) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

The compounds of the present invention are also of value in inhibiting the entry of viruses (such as human immunodeficiency virus (HIV)) into target calls and, therefore, are of value in the prevention of infection by viruses (such as HIV), the treatment of infection by viruses (such as HIV) and the prevention and/or treatment of acquired immune deficiency syndrome (AIDS).

According to a further feature of the invention there is provided a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof, for use in a method of treatment of a warm blooded animal (such as man) by therapy (including prophylaxis).

According to a further feature of the present invention there is provided a method for modulating chemokine receptor activity (such as CCR5 receptor activity) in a warm blooded animal, such as man, in need of such treatment, which comprises administering to said animal an effective amount of a compound of the present invention, or a pharmaceutically acceptable salt thereof or a solvate thereof.

The present invention also provides the use of a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof, as a medicament, such as a medicament for the treatment of transplant rejection, respiratory disease, psoriasis or rheumatoid arthritis (such as rheumatoid arthritis). [Respiratory disease is, for example, COPD, asthma (such as bronchial, allergic, intrinsic, extrinsic or dust asthma, particularly chronic or inveterate asthma (for example late asthma or airways hyper-responsiveness)} or rhinitis {acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis}; and is particularly asthma or rhinitis].

In another aspect the present invention provides the use of a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof, in the manufacture of a medicament for use in therapy (for example modulating chemokine receptor activity (such as CCR5 receptor activity (such as rheumatoid arthritis)) in a warm blooded animal, such as man).

The invention also provides a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof, for use as a medicament, such as a medicament for the treatment of rheumatoid arritis.

In another aspect the present invention provides the use of a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof, in the manufacture of a medicament for use in therapy (for example modulating chemokine receptor activity (such as CCR5 receptor activity (such as rheumatoid arthritis)) in a warm blooded animal, such as man).

The invention further provides the use of a compound of formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof, in the manufacture of a medicament for use in the treatment of:
(1) (the respiratory tract) obstructive diseases of airways including: chronic obstructive pulmonary disease (COPD) (such as irreversible COPD); asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertrophic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behcet's disease, Sjogren's syndrome or systemic sclerosis;
(3) (skin and eyes) psoriasis, atopic dermatitis, contact dermatitis or other eczmatous dermitides, seborrhoetic dermatitis, Lichen planus, Phemphigus, bullous Phemphigus, Epidermolysis bullosa, urticaria, angiodermas, vasculitides erythemas, cutaneous eosinophilias, uveitis, Alopecia areata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allograft rejection) acute and chronic following, for example, transplantation of kidney, heart, liver, lung, bone marrow, skin or cornea; or chronic graft versus host disease; and/or
(6) (other tissues or diseases) Alzheimer's disease, multiple sclerosis, atherosclerosis, Acquired Immunodeficiency Syndrome (AIDS), Lupus disorders (such as lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura or disorders of the menstrual cycle;
   in a warm blooded animal, such as man.

The present invention further provides a method of treating a chemokine mediated disease state (such as a CCR5 mediated disease state) in a warm blooded animal, such as man, which comprises administering to a mammal in need of such treatment an effective amount of a compound of formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or solvate thereof.

In order to use a compound of the invention, or a pharmaceutically acceptable salt thereof or solvate thereof, for the therapeutic treatment of a warm blooded animal, such as man, in particular modulating chemokine receptor (for example CCR5 receptor) activity, said ingredient is normally formulated in accordance with standard pharmaceutical practice as a pharmaceutical composition.

Therefore in another aspect the present invention provides a pharmaceutical composition which comprises a compound of the formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvate thereof (active ingredient), and a pharmaceutically acceptable adjuvant, diluent or carrier. In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (per cent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible

powders, suppositories, ointments, creams, drops and sterile injectable aqueous or oily solutions or suspensions.

A suitable pharmaceutical composition of this invention is one suitable for oral administration in unit dosage form, for example a tablet or capsule which contains between 0.1 mg and 1 g of active ingredient.

In another aspect a pharmaceutical composition of the invention is one suitable for intravenous, subcutaneous or intramuscular injection.

Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose which is approximately equivalent to the daily parenteral dose, the composition being administered 1 to 4 times per day.

The following illustrate representative pharmaceutical dosage forms containing the compound of formula (I), (Ia), (Ib), (Ic), (Id), (Ie), (If), (Ig), (Ih), (Ii), (Ij), (Ik), (Il), (Im) or (In) (for example a compound of formula (I), (Ia), (Ib), (Ic), (Id) or (Ie)), or a pharmaceutically acceptable salt thereof or a solvent thereof (hereafter Compound X), for therapeutic or prophylactic use in humans:

(a)

| Tablet I | mg/tablet |
|---|---|
| Compound X | 100 |
| Lactose Ph. Eur. | 179 |
| Croscarmellose sodium | 12.0 |
| Polyvinylpyrrolidone | 6 |
| Magnesium stearate | 3.0 |

(b)

| Tablet II | mg/tablet |
|---|---|
| Compound X | 50 |
| Lactose Ph. Eur. | 229 |
| Croscarmellose sodium | 12.0 |
| Polyvinylpyrrolidone | 6 |
| Magnesium stearate | 3.0 |

(c)

| Tablet III | mg/tablet |
|---|---|
| Compound X | 1.0 |
| Lactose Ph. Eur. | 92 |
| Croscarmellose sodium | 4.0 |
| Polyvinylpyrrolidone | 2.0 |
| Magnesium stearate | 1.0 |

(d)

| Capsule | mg/capsule |
|---|---|
| Compound X | 10 |
| Lactose Ph. Eur. | 389 |
| Croscarmellose sodium | 100 |
| Magnesium stearate | 1.0 |

(e)

| Injection I | (50 mg/ml) |
|---|---|
| Compound X | 5.0% w/v |
| Isotonic aqueous solution | to 100% |

Buffers, pharmaceutically-acceptable cosolvents such as polyethylene glycol, polypropylene glycol, glycerol or ethanol or complexing agents such as hydroxy-propyl β-cyclodextrin may be used to aid formulation.

The above formulations may be obtained by conventional procedures well known in the pharmaceutical art. The tablets (a)-(c) may be enteric coated by conventional means, for example to provide a coating of cellulose acetate phthalate.

The invention further relates to combination therapies or compositions wherein a compound of formula (I), or a pharmaceutically acceptable salt, solvate or a solvate of a salt thereof, or a pharmaceutical composition comprising a compound of formula (I), or a pharmaceutically acceptable salt, solvate or a solvate of a salt thereof, is administered concurrently (possibly in the same composition) or sequentially with an agent for the treatment of any one of the above disease states.

In particular, for the treatment of the inflammatory diseases rheumatoid arthritis, psoriasis, inflammatory bowel disease, COPD, asthma and allergic rhinitis a compound of the invention can be combined with a TNF-α inhibitor (such as an anti-TNF monoclonal antibody (such as Remicade, CDP-870 and D.sub2.E.sub7.), or a TNF receptor immunoglobulin molecule (such as Enbrel.reg.)), a non-selective COX-1/COX-2 inhibitor (such as piroxicam or diclofenac; a propionic acid such as naproxen, flubiprofen, fenoprofen, ketoprofen or ibuprofen; a fenamate such as mefenamic acid, indomethacin, sulindac or apazone; a pyrazolone such as phenylbutazone; or a salicylate such as aspirin), a COX-2 inhibitor (such as meloxicam, celecoxib, rofecoxib, valdecoxib or etoricoxib) low dose methotrexate, lefunomide; ciclesonide; hydroxychloroquine, d-penicillamine or auranofin, or parenteral or oral gold.

The present invention still further relates to the combination of a compound of the invention together with:

a leukotriene biosynthesis inhibitor, a 5-lipoxygenase (5-LO) inhibitor or a 5-lipoxygenase activating protein (FLAP) antagonist, such as zileuton, ABT-761, fenleuton, tepoxalin, Abbott-79175, Abbott-85761, an N-(5-substituted)-thiophene-2-alkylsulfonamide, a 2,6-di-tert-butylphenol hydrazones, a methoxytetrahydropyran such as Zeneca ZD-2138, SB-210661, a pyridinyl-substituted 2-cyanonaphthalene compound such as L-739,010; a 2-cyanoquinoline compound such as L-746,530; an indole or quinoline compound such as MK-591, MK-886 or BAY x 1005;

a receptor antagonist for a leukotriene LTB.sub4., LTC.sub4., LTD.sub4. or LTE.sub4. selected from the group consisting of a phenothiazin-3-one such as L-651,392; an amidino compound such as CGS-25019c; a benzoxalamine such as ontazolast; a benzenecarboximidamide such as BIIL 284/260; or a compound such as zafirlukast, ablukast, montelukast, pranlukast, verlukast (MK-679), RG-12525, Ro-245913, iralukast (CGP 45715A) or BAY x 7195;

a PDE4 inhibitor including an inhibitor of the isoform PDE4D;

an antihistaminic H.sub1. receptor antagonist such as cetirizine, loratadine, desloratadine, fexofenadine, astemizole, azelastine or chlorpheniramine;

a gastroprotective H.sub2. receptor antagonist;

an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride or ethylnorepinephrine hydrochloride;

an anticholinergic agent such as ipratropium bromide, tiotropium bromide, oxitropium bromide, pirenzepine or telenzepine;

a βB.sub1.- to β.sub4.-adrenoceptor agonist such as metaproterenol, isoproterenol, isoprenaline, albuterol, salbutamol, formoterol, salmeterol, terbutaline, orciprenaline, bitolterol mesylate or pirbuterol, or a methylxanthanine including theophylline and aminophylline; sodium cromoglycate; or a muscarinic receptor (M1, M2, and M3) antagonist;

an insulin-like growth factor type I (IGF-1) mimetic;

an inhaled glucocorticoid with reduced systemic side effects, such as prednisone, prednisolone, flunisolide, triamncinolone acetonide, beclomethasone dipropionate, budesonide, fluticasone propionate or mometasone furoate;

an inhibitor of a matrix metalloprotease (MMP), such as a stromelysin, a collagenase, or a gelatinase or aggrecanase; such as collagenase-1 (MMP-1), collagenase-2 (MMP-8), collagenase-3 (MMP-13), stromelysin-1 (MMP-3), stromelysin-2 (MMP-10), and stromelysin-3 (MMP-11) or MMP-12;

a modulator of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family;

an osteoporosis agent such as roloxifene, droloxifene, lasofoxifene or fosomax;

an immunosuppressant agent such as FK-506, rapamycin, cyclosporine, azathioprine or methotrexate;

a compound useful in the treatment of AIDS and/or HIV infection for example: an agent which prevents or inhibits the viral protein gp120 from engaging host cell CD4 {such as soluble CD4 (recombinant); an anti-CD4 antibody (or modified/recombinant antibody) for example PRO542; an anti-group120 antibody (or modified/recombinant antibody); or another agent which interferes with the binding of group120 to CD4 for example BMS806}; an agent which prevents binding to a chemokine receptor, other than CCR5, used by the HIV virus {such as a CXCR4 agonist or antagonist or an anti-CXCR4 antibody}; a compound which interferes in the fusion between the HIV viral envelope and a cell membrane {such as an anti-group 41 antibody; enfuvirtide (T-20) or T-1249}; an inhibitor of DC-SIGN (also known as CD209) {such as an anti-DC-SIGN antibody or an inhibitor of DC-SIGN binding}; a nucleoside/nucleotide analogue reverse transciptase inhibitor {for example zidovudine (AZT), nevirapine, didanosine (ddI), zalcitabine (ddC), stavudine (d4T), lamivudine (3TC), abacavir, adefovir or tenofovir (for example as free base or as disoproxil fumarate)}; a non-nucleoside reverse transciptase inhibitor {for example nevirapine, delavirdine or efavirenz}; a protease inhibitor {for example ritonavir, indinavir, saquinavir (for example as free base or as mesylate salt), nelfinavir (for example as free base or as mesylate salt), amprenavir, lopinavir or atazanavir (for example as free base or as sulphate salt)}; a ribonucleotide reductase inhinbitor {for example hydroxyurea}; or an antiretroviral {for example emtricitabine}; or,

an existing therapeutic agent for the treatment of osteoarthritis, for example a non-steroidal anti-inflammatory agent (hereinafter NSAID's) such as piroxicam or diclofenac, a propionic acid such as naproxen, flubiprofen, fenoprofen, ketoprofen or ibuprofen, a fenamate such as mefenamic acid, indomethacin, sulindac or apazone, a pyrazolone such as phenylbutazone, a salicylate such as aspirin, a COX-2 inhibitor such as celecoxib, valdecoxib, rofecoxib or etoricoxib, an analgesic or intra-articular therapy such as a corticosteroid or a hyaluronic acid such as hyalgan or synvisc, or a P2X7 receptor antagonist.

The present invention still further relates to the combination of a compound of the invention together with: (i) a tryptase inhibitor; (ii) a platelet activating factor (PAF) antagonist; (iii) an interleukin converting enzyme (ICE) inhibitor; (iv) an IMPDH inhibitor; (v) an adhesion molecule inhibitor including a VLA-4 antagonist; (vi) a cathepsin; (vii) a MAP kinase inhibitor; (viii) a glucose-6 phosphate dehydrogenase inhibitor; (ix) a kinin-B.sub1.- and B.sub2.-receptor antagonist; (x) an anti-gout agent, e.g., colchicine; (xi) a xanthine oxidase inhibitor, e.g., allopurinol; (xii) an uricosuric agent, e.g., probenecid, sulfinpyrazone or benzbromarone; (xiii) a growth hormone secretagogue; (xiv) a transforming growth factor (TGFβ); (xv) a platelet-derived growth factor (PDGF); (xvi) a fibroblast growth factor, e.g., basic fibroblast growth factor (bFGF); (xvii) a granulocyte macrophage colony stimulating factor (GM-CSF); (xviii) a capsaicin cream; (xix) a Tachykinin NK.sub1. and NK-sub3. receptor antagonist selected from the group consisting of NKP-608C; SB-233412 (talnetant); and D-4418; (xx) an elastase inhibitors selected from the group consisting of UT-77 and ZD-0892; (xxi) a TNFα converting enzyme inhibitor (TACE); (xxii) an induced nitric oxide synthase inhibitor (iNOS); or (xxiii) a chemoattractant receptor-homologous molecule expressed on TH2 cells (a CRTH2 antagonist).

The invention will now be illustrated by the following non-limiting Examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulfate; evaporation of solvent was carried out using a rotary evaporator under reduced pressure (600-4000 Pascals; 4.5-30 mm Hg) with a bath temperature of up to 60° C.;
(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where “Argonaut™ PS-tris-amine scavenger resin” is referred to, this means a tris-(2-aminoethyl)amine polystyrene resin obtained from Argonaut Technologies Inc., 887 Industrial Road, Suite G, San Carlos, Calif., USA.
(iv) in general, the course of reactions was followed by TLC and reaction times are given for illustration only;
(v) yields, when given, are for illustration only and are not necessarily those which can be obtained by diligent process development; preparations were repeated if more material was required;
(vi) when given, ¹H NMR data is quoted and is in the form of delta values for major diagnostic protons, given in parts per million (ppm) relative to tetramethylsilane (TMS) as an internal standard, determined at 300 MHz using perdeuterio DMSO (CD₃SOCD₃) as the solvent unless otherwise stated; coupling constants (J) are given in Hz;
(vii) chemical symbols have their usual meanings; SI units and symbols are used;
(viii) solvent ratios are given in percentage by volume;
(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;
(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid. The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺;
(xi) PS-NCO resin is an isocyanate resin and is available from Argonaut;
(xii) Powder X-Ray Diffractometry (PXRD) analyses were performed using a Siemens D5000. The X-ray powder diffraction spectra were determined by mounting a sample of the crystalline salt on Siemens single silicon crystal (SSC) wafer mounts and spreading out the sample into a thin layer with the aid of a microscope slide. The sample was spun at 30 revolutions per minute (to improve counting statistics) and irradiated with X-rays generated by a copper long-fine focus tube operated at 40 kV and 40 mA with a wavelength of 1.5406 angstroms. The collimated X-ray source was passed through an automatic variable divergence slit set at V20 and the reflected radiation directed through a 2 mm antiscatter slit and a 0.2 mm detector slit. The sample was exposed for 1 second per 0.02 degree 2-theta increment (continuous scan mode) over the range 2 degrees to 40 degrees 2-theta in theta-theta mode. The running time was 31 minutes and 41 seconds. The instrument was equipped with a scintillation counter as detector. Control and data capture was by means of a Dell Optiplex 686 NT 4.0 Workstation operating with Diffract+ software. Persons skilled in the art of X-ray powder diffraction will realise that the relative intensity of peaks can be affected by, for example, grains above 30 microns in size and non-unitary aspect ratios which may affect analysis of samples. The skilled person will also realise that the position of reflections can be affected by the precise height at which the sample sits in the diffractometer and the zero calibration of the diffractometer. The surface planarity of the sample may also have a small effect.; and,

(xiii) the following abbreviations are used:

| | |
|---|---|
| THF | tetrahydrofuran; |
| Boc | tert-butoxycarbonyl |
| DMF | N,N-dimethylformamide |
| DCM | dichloromethane |
| DIPEA | N,N-Diisopropylethylamine |
| R-BINAP | R2,2′-Bis(diphenylphosphino)-1,1′-binaphthyl |
| HATU | O-(7-Azabenzotriazol-1-yl)-N,N,N′,N′- |
| | tetramethyluronium hexafluorophosphate |
| EDCI | ethyl dimethylaminopropyl carbodiimide |
| HOBT | 1-hydroxybenzotriazole |

### EXAMPLE 1

### This Example Illustrates the Preparation of N-(3-phenyl-3-[4-methanesulphonylpiperazin-1-yl]propyl)-4-[2-(4-methanesulphonylphenylsulphonyl)ethyl]-piperidine (Compound No. 8, Table I)

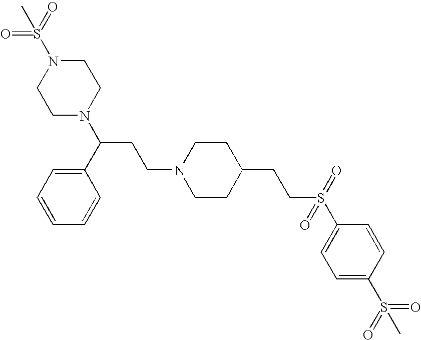

N-(3-Phenyl-3-chloropropyl)-4-[2-(4-methanesulphonylphenylsulphonyl)ethyl]-piperidine (prepared according to Method D; 180 mg) was added to a solution of N-methanesulphonylpiperazine (61 mg) and triethylamine (0.102 ml) in dichloromethane (10 ml) and the mixture was allowed to stand at room temperature for 16 hours. The reaction mixture was poured onto a 20 g silica Bond Elut eluted with a solvent gradient (ethyl acetate—25% methanol/ethyl acetate). The title compound was obtained, yield 67 mg, MH⁺612.

NMR (CDCl₃): 1.6-1.8 (m, 7H), 2.2-2.6(m, 9H), 2.7(m, 1H), 2.75 (s, 3H), 3.2 (m, 11H), 3.45 (m, 1H), 7.2 (d, 2H), 7.3 (m, 3H), 8.2 (m, 4H).

### EXAMPLE 2

### This Example Illustrates the Preparation of N-(3-phenyl-3-[1-methanesulphonyl-piperidin-4-yl]propyl)-4-[2-(4-fluorophenylsulphonyl)ethyl]piperidine (Compound No. 10, Table I)

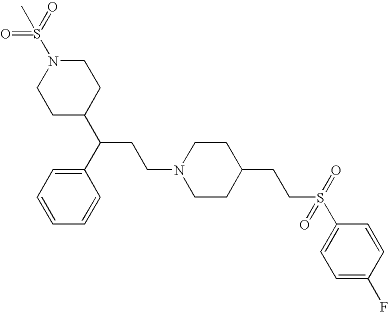

Sodium triacetoxyborohydride (267 mg) was added to a mixture of 3-(1-methanesulphonylpiperidin-4-yl)-3-phenylpropionaldehyde (247 mg) and 4-(2-[4-fluorophenylsulphonyl]ethyl)piperidine hydrochloride salt (288 mg) (CAS 313994-09-1) in dichloromethane (20 ml) and the mixture was stirred for 16 hours. The reaction mixture was washed successively with 2M sodium hydroxide (10 ml), water (10 ml) and brine (10 ml) and was dried. The residue obtained on removal of the solvent was chromatographed on a 20 g silica Bond Elut column eluting with a solvent gradient (ethyl acetate—20% methanol/ethyl acetate) to give the title compound, yield 250 mg, MH⁺551.

NMR (CDCl₃): 1.2 (m, 5H), 1.4 (m, 4H), 1.6-1.8 (m, 8H), 2.0 (m, 3H), 2.4 (m, 1H), 2.5-2.6 (m, 2H), 2.8 (s, 3H), 2.85 (m, 2H), 3.1 (m, 2H), 3.7 (d, 1 H), 3.8 (d, 1H), 7.1 (m, 2H), 7.3 (m, 5H), 7.9 (m, 2H).

### EXAMPLE 3

### This Example Illustrates the Preparation of (S) N-(3-phenyl-3-[4-chlorobenzoyl-amino]propyl-4-[2-(4-methanesulphonylphenylsulphonyl)ethyl]piperidine (Compound No. 2, Table IV)

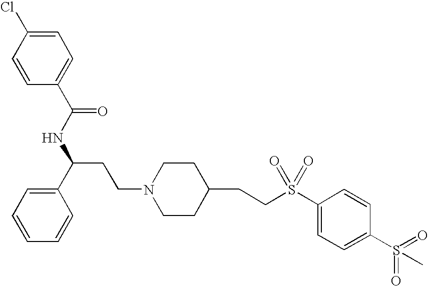

4-Chlorobenzoyl chloride (76 μl) was added to a solution of (S) N-(3-amino-3-phenylpropyl)-4-[2-(4-methanesulphonylphenylsulphonyl)ethyl]piperidine (280 mg) and triethylamine (157 μw) in dichloromethane (15 ml) and the mixture was stirred for 1 hour then washed with water (15 ml) and brine (15 ml) and dried. Removal of the solvent gave the title compound as a white solid, yield 320 mg, MH⁺602.

NMR (d6 DMSO): 1.0 (m, 2H), 1.2 (m, 1H), 1.5 (m, 2H), 1.6 (m, 2H), 1.8 (m, 2H), 1.9 (m, 2H), 2.25 (m, 2H), 2.8 (m, 2H), 3.3 (m,3H), 3.4 m, 2H), 5.0 (q, 1H), 7.2 (m, 1H), 7.3 (m,3H), 7.5 (d, 2H), 7.85 (d, 2H), 8.2 (m, 4H), 8.9 (d, 1H).

### EXAMPLE 4

### This Example Illustrates the Preparation of 1-{(3R)-3-(3,5-difluorophenyl)-3-[4-(methylsulfonyl)phenyl]propyl}-4-(2- {[4-(methylsulfonyl)phenyl]sulfonyl}ethyl)piperidine (Compound No. 7, Table V) resin (1.34 g), PS-isocyanate resin (682 mg) and PS-thiophenol resin (577 mg) were added. The mixture was stirred for 5 hours, filtered and the resins were washed with 10% methanol in dichloromethane (2×25 ml). The combined filtrates were evaporated to dryness and the residue was passed through a 20 g Isolute column eluted with a solvent gradient of ethyl acetate-10% methanol/ethyl acetate to give the title compound, yield 81 mg; MH⁺552.

NMR (CDCl₃): 1.12-1.32 (m, 4H), 1.52-1.66 (m, 4H), 1.76-1.93 (m, 3H), 2.08 (m, 1H), 2.21 (m, 1H), 2.47-2.51 (m, 4H), 2.71 (s, 3H), 2.77-2.88 (m, 2H), 3.03-3.10 (m, 3.12-3.21 (m, 4H), 3.37 (m, 1H), 7.14 (d, 2H), 7.15-7.32 (m, 5H), 7.88 (m, 2H).

### EXAMPLE 6

### This Example Illustrates the Preparation of (R) N-(3-[3,5-difluorophenyl]-3-[1-methanesulphonylpiperdin-4-yl]propyl)-4-[2-(4-methanesulphonylphenylsulphonyl)-ethyl]piperidine (Compound 32 in Table III)

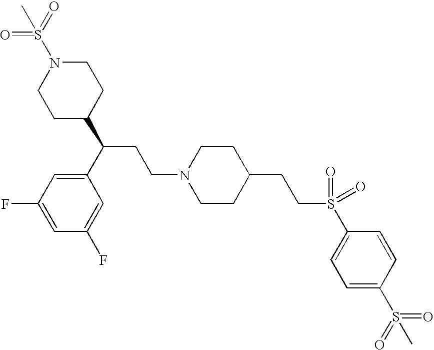

MP-triacetoxyborohydride (where MP stands for “macroporous”; 585 mg) was added to a solution of (R) 3-(1-methanesulphonylpiperidin-4-yl)-3-[3,5-difluorophenyl]propionaldehyde (199 mg) (Method G) and 4-(2-[4-methanesulphonylphenylsulphonyl]ethyl)piperidine (194 mg) (Method B) in 20 ml of dichloromethane and the mixture was stirred at room temperature for 16 hours. The reaction mixture was filtered and the solid washed with dichloromethane (3×10 ml) and the combined dichloromethane filtrate and washings were poured onto a 25 g bond Elut cartridge and eluted with a solvent gradient (ethyl acetate—20% methanol/ethyl acetate) to give the title compound, yield 168 mg; MH⁺647. NMR DMSOd6) [note not all peaks are reported]: 2.78 (s, 3H), 6.87 (d, 2H), 6.99 (t, 1H), 8.14 (q, 4H).

### EXAMPLE 7

This Example illustrates the preparation of (R) N-(3-phenyl-3-[1-methanesulphonylpiperidin-4-yl]propyl)-4-[2-(4-methanesulphonyloxyphenylsulphonyl)-ethyl]piperidine (Compound 29 in Table III).

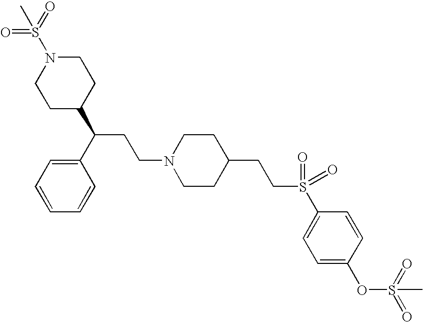

Methanesulphonyl chloride (60.3 mg) was added to a solution of (R) N-(3-phenyl-3-[1-methanesulphonylpiperidin-4-yl]propyl)-4-[2-(4-hydroxyphenylsulphonyl)ethyl]piperidine (Compound 28 in Table III; 290 mg) and triethylamine (53 mg) in dichloromethane (10 ml) and the mixture was stirred for 16 hours, then washed with saturated aqueous sodium bicarbonate (2×20 ml) and dried. The drying agent was filtered and the filtrate was poured onto a 20 g Bond Elut cartridge and eluted with a solvent gradient (ethyl acetate—20% methanol/ethyl acetate) to give the product, yield 41.5 mg. NMR (D)MSOd6) [note not all peaks are reported]: δ2.77 (s, 3H), 7.11-7.23 (m, 3H), 7.30 (t, 2H), 7.60 (d, 2H), 8.0 (d, 2H).

### EXAMPLE 8

This Example illustrates the preparation of (R) N-(3-phenyl-3-[1-methanesulphonyl-piperidin-4-yl]propyl)-4-[2-(4-tetrazol-5-yl-phenylsulphonyl)ethyl]piperidine (Compound 30 in Table III).

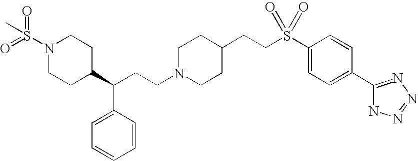

Ammonium chloride (67 mg) and sodium azide (81.6 mg) were added to a solution of (R) N-(3-phenyl-3-[1-methanesulphonylpiperidin-4-yl]propyl)-4-[2-(4-cyanophenyl-sulphonyl)ethyl]piperidine (350 mg; prepared by the method described in Example 6 using 4-(2-[4-cyanophenylsulphonyl]ethyl)piperidine [Method B] as reactant) in DMF (10 ml) and the mixture was heated at 100° C. for 8 hours. Further equivalents of ammonium chloride (67 mg) and sodium azide (81.6 mg) were added and the mixture was heated at 100° C. for a further 8 hours. The solvent was evaporated and the residue was stirred with water (10 ml). The water was decanted and the residue was dissolved in methanol (10 ml) and poured on to a 20 g SCX2 cartridge eluted with methanol (4×20 ml) and 1M ammonia/methanol. The ammonia/methanol washings were evaporated to dryness to give the title compound, yield 140 mg, MH⁺ 601. NMR (DMSOd6) [note not all peaks are reported]: δ2.77 (s, 3H), 7.04-7.25 (m, 3H), 7.30 (t, 2H), 7.85 (d, 2H), 8.18 (d, 2H).

### EXAMPLE 9

### Preparation of (4-{[2-(1-{(3R)-3-(3,5-difluorophenyl)-3-[4-(methylsulfonyl)phenyl]propyl}piperidin-4-yl)ethyl]sulfonyl}phenoxy)acetonitrile (Compound 15 of Table V) and 2-(4-{[2-(1-{(3R)-3-(3,5-difluorophenyl)-3-[4-(methylsulfonyl)phenyl]propyl}piperidin-4-yl)ethyl]sulfonyl}phenoxy)acetamide (Compound 16 of Table V ).

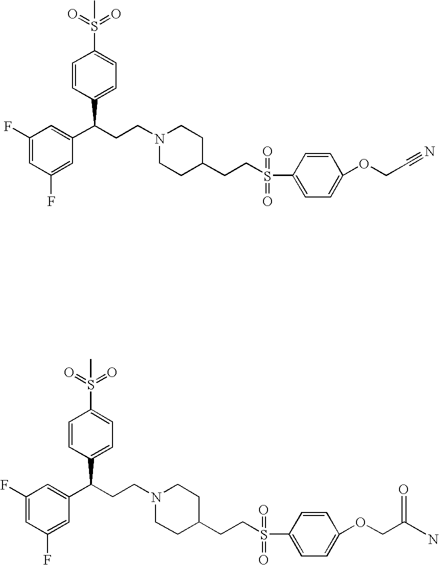

{4-[(2-Piperidinmylethyl)sulfonyl]phenoxy}acetonitrile (0.9 g, Method M) was dissolved in a solution of (R)-3-(3,5-difluorophenyl)-3-(4-methanesulphonylphenyl)-propionaldehyde (0.85 g) in dichloromethane (50 ml) and sodium triacetoxyborohydride (0.55 g) was added. The reaction mixture was stirred for 16 hours, washed with 2M NaOH (2×50 ml), dried and evaporated to dryness. The residue obtained was purified by chromatography on a Bond-Elut column using an elution gradient of ethyl acetate—30% methanol/ethyl acetate to give the title compound, yield 370 mg.

NMR (DMSOd6): 0.9-1.8 (m, 10H), 2-2.3 (m, 5H), 2.7 (m, 2H), 3.1 (s, 3H), 4.2 (t, 1H), 5.3 (s, 2H), 6.9-7.2 (m, 5H), 7.5-7.9 (m, 6H); M⁺H 617.

A second fraction was collected and shown to be 2-(4-{[2-(1-{(3R)-3-(3,5-difluorophenyl)-3-[4-(methylsulfonyl)phenyl]propyl}piperidin-4-yl)ethyl]sulfonyl}phenoxy)acetamide (Compound 16 of Table V), yield 168 mg.

NMR (DMSOd6): 0.9-1.8 (m, 10H), 2-2.3 (m, 5H), 2.7 (m, 2H), 3.1 (s, 3H), 4.2 (t, 1H), 4.65 (s, 2H), 6.9-7.2 (m, 5H), 7.4-7.9 (m, 6H); M⁺H 635.

### EXAMPLE 10

### Preparation of 1-{(3R)-3-(3,5-difluorophenyl)-3-[4-(methylsulfonyl)phenyl]propyl}-4-(2-{[4-(1H-tetrazol-5-ylmethoxy)phenyl]sulfonyl}ethyl)piperidine (Compound 17 of Table V)

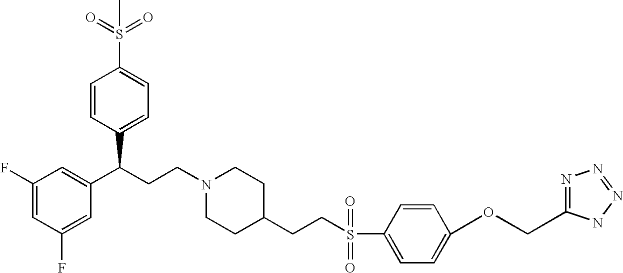

A mixture of (4-{[2-(1-{(3R)-3-(3,5-difluorophenyl)-3-[4-(methylsulfonyl)phenyl]-propyl}piperidin4-yl)ethyl]sulfonyl}phenoxy)acetonitrile (300 mg), sodium azide (63 mg) and ammonium chloride (52 mg) in DMF (10 ml) was stirred and heated at 100° C. for 4 hours. The solvent was evaporated and the residue was dissolved in water (10 ml). Water was decanted from the oil obtained and the residual oil was dissolved in methanol (10 ml) and poured onto a 20 g SCX2 cartridge and eluted with methanol (4×20 ml) and 1M ammonia/methanol (5×20 ml). The methanolic ammonia washings were evaporated to give the title compound, yield 0.15 g. M⁺H 660. NMR (DMSOd6) [note not all peaks are reported]: δ 5.3 (s, 2H), 7.04 (t, 1H), 7.09-7.18 (m, 2H), 7.26 (d, 2H), 7.59 (d, 2H), 7.75 (d, 2H), 7.84 (d, 2H).

### EXAMPLE 11

### Preparation of 1-{(3R)-3-(3,5-difluorophenyl)-3-[4-(methylsulfonyl)phenyl]propyl}-4-(2-{[4-(2-methyl-2H-tetrazol-5-yl)phenyl]sulfonyl}ethyl)piperidine (Compound 19 of Table V)

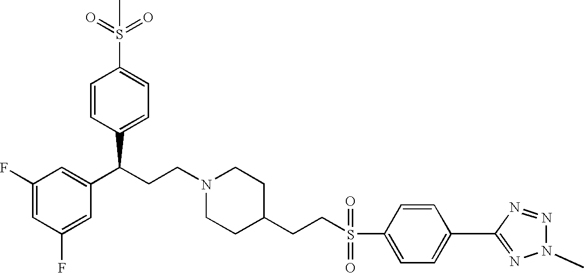

4-(2-{[4-(2-methyl-2H-tetrazol-5-yl)phenyl]sulfonyl}ethyl)piperidine (300 mg, Method N) was added to a solution of (R)-3-(3,5-difluorophenyl)-3-(4-methanesulphonyl-phenyl)propionaldehyde (290 mg) dissolved in dichloromethane (20 ml) followed by MP-triacetoxyborohydride (900 mg) and the reaction mixture was stirred for 16 hours, filtered and evaporated to dryness. The residue was purified by chromatography on a Bond-elut column using an eluant gradient of ethyl acetate—15% methanol/ethyl acetate to give the title compound, yield 167 mg.

NMR (CDCl₃): 1.2-1.4 (m, 3H), 1.6-1.9 (m, 7H), 2.8 (m, 2H), 3.05 (s, 3H), 3.1 (m, 2H), 4.1 (m, 1H), 4.05 (s, 3H), 6.7 (m, 3H), 7.4 (m, 2H), 7.85 (d, 2H), 8 (d, 2H), 8.39 (d, 2H), M⁺H 644.

Using the procedure outlined above and using (R)-3-(1-methanesulphonylpiperidin-4-yl)-3-phenylpropionaldehyde (Method G) as starting material there was obtained 1-(methylsulfonyl)-4-{(1R)-3-[4-(2-{[4-(2-methyl-2H-tetrazol-5-yl)phenyl]sulfonyl}ethyl)-piperidin-1-yl]-1-phenylpropyl}piperidine (Compound 20 of Table V), M⁺H 615.

### EXAMPLE 12

### Preparation of methyl (4-{[2-1-{(3R)-3-(3,5-difluorophenyl)-3-[4-(methylsulfonyl)phenyl]propyl}piperidin-4-yl)ethyl]sulfonyl}phenoxy)acetate (Compound 27 of Table V)

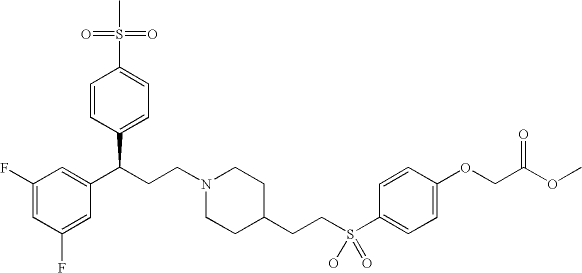

The product obtained on the reductive amination of (3R)-3-(3,5-difluorophenyl)-3-[4-(methylsulfonyl)phenyl]propanal (0.85 g) with benzyl {4-[(2-piperidin-4-ylethyl)sulfonyl]phenoxy}acetate (1.1 g; prepared according to Method M steps 1 and 2, using benzyl bromoacetate as starting material), carried out according to the method described in Example 4, was poured onto a 20 g SCX2 column and eluted with methanol (5×20 ml) and 10% ammonia in methanol (5×20 ml). The methanolic ammonia washings were concentrated and the product isolated had undergone ester exchange with the methanol eluant. The title compound was obtained, yield 1.3g; M⁺H 650.

NMR (CDCl₃): 1.2 (m, 4H), 1.6 (m, 6H), 1.8 (t, 2H), 2.8 (m, 2H), 3.01 (s, 3H), 3.1 (m, 2H), 3.8 (s, 3H), 4.1 (m, 1H), 4.7 (s, 2H), 6.6-6.8 (m, 3H), 7 (d, 2H), 7.4 (d, 2H), 7.8-7.9 (m, 4H).

### EXAMPLE 13

### Preparation of (4-{[2-(1-{(3R)-3-(3,5-difluorophenyl)-3-[4-(methylsulfonyl)phenyl]-propyl}piperidin-4-yl)ethyl]sulfonyl}phenoxy)acetic acid (Compound 28 of Table V).

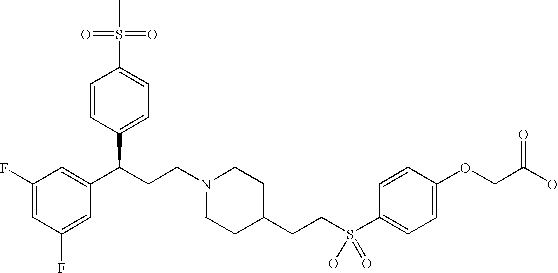

2M NaOH was added to a solution of methyl (4-{[2-(1-{(3R)-3-(3,5-difluorophenyl)-3-[4-(methylsulfonyl)phenyl]propyl}piperidin-4-yl)ethyl]sulfonyl}phenoxy)acetate (1.2 g) (Example 12) in a mixture of ethanol (20 ml) and THF (20 ml) and the mixture was stirred for 2 hours. The reaction mixture was evaporated to dryness and water (10 ml) was added. The solution was acidified to pH 3 with 2M HCl, the pH was adjusted to ˜5 with sodium acetate and the mixture was extracted with dichloromethame (4×25 ml). The combined extracts were dried and evaporated to give the title compound, yield 0.9 g. M⁺H 636.

NMR (CDCl₃) [note that not all peaks are reported]: 3.03 (s, 3H), 4.01 (t, 1H), 4.48 (bs, 2H), 6.67 (t, 1H), 6.73 (d, 2H), 7.40 (d, 2H), 7.73 (d, 2H), 7.86 (d, 2H).

### EXAMPLE 14

### Preparation of 2-(4-{[2-(1-{(3R)-3-(3,5-difluorophenyl)-3-[4-(methylsulfonyl)phenyl]propyl}piperidin-4-yl)ethyl]sulfonyl}phenoxy)-N-(methylsulfonyl)acetamide (Compound 29 Table V)

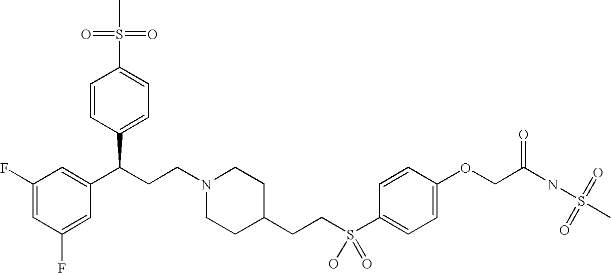

EDCI was added to a solution of (4-{[2-(1-{(3R)-3-(3,5-difluorophenyl)-3-[4-(methylsulfonyl)phenyl]propyl}piperidin-4-yl)ethyl]sulfonyl}phenoxy)acetic acid (400 mg), methanesulphonamide (59 mg) and dimethylaminipyridine (163 mg) in dichloromethane and the mixture was stirred for 20 hours. The mixture was washed with water (2×25 ml), dried and evaporated to dryness. The residue was passed through a Bond-Elut column eluting with a solvent gradient of ethyl acetate—25% methanol/ethyl acetate to give the title compound as a white solid, yield 8.3 mg. M⁺H 713.

NMR (DMSOd6): [note that not all peaks are reported]: 4.18 (t, 1H), 4.42 (s, 2H), 6.97-7.12 (m, 3H), 7.15 (d, 2H), 7.59 (d, 2H), 7.74 (d, 2H), 7.85 (d, 2H).

### EXAMPLE 15

### Preparation of 2-(4-{[2-(1-{(3R)-3-(3,5-difluorophenyl)-3-[4-(methylsulfonyl)phenyl]propyl}piperidin-4-yl)ethyl]sulfonyl}phenoxy)-N-methylacetamide (Compound 30 Table V)

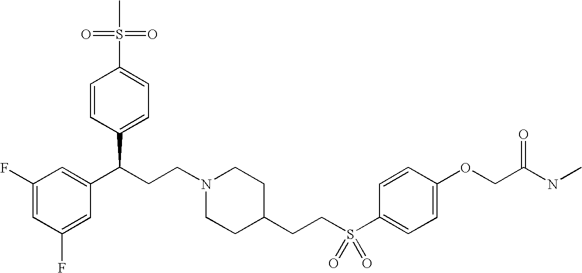

A mixture of (4-{[2-(1-{(3R)-3-(3,5-difluorophenyl)-3-[4-(methylsulfonyl)phenyl]-propyl}piperidin-4-yl)ethyl]sulfonyl}phenoxy)acetic acid (400 mg), HOBT (85 mg) and EDCI (245 mg) in dichlordmethane (25 ml) was stirred at room temperature for 1.5 hours. Methanolic ammonia (10 ml) was added and stirring was continued for 16 hours. The reaction mixture was washed with water (2×20 ml), dried and evaporated to dryness and the residue obtained was dissolved in dichloromethane (20 ml) and stireed with MP carbonate (1 g) for 2 hours. The product was chromatograhed on a Bond-Elut column eluting with a solvent gradient of ethyl acetate—10% methanol/ethyl acetate to give the title compound, yield 144 mg. M⁺H 649.

NMR (CDCl₃): 1.2 (m, 4H), 1.6 (m, 6H), 1.8 (t, 2H), 2.8 (m, 2H), 2.95 (d, 3H), 3.01 (s, 3H), 3.1 (m, 2H), 4.1 (m, 1H), 4.6 (s, 2H), 6.5 (broad peak, 1H), 6.6-6.8 (m, 3H), 7.05 (d, 2H), 7.4(d, 2H), 7.9 (m, 4H).

### EXAMPLE 16

### Preparation of 1-{(1S,3(R or S)-3-(3,5-difluorophenyl)-1-methyl-3-(4-(methylsulfonyl)phenyl]propyl}-4-(2-{[4-(methylsulfonyl)phenyl]sulfonyl}ethyl)piperidine and 1-{(1S,3(S or R)-3-(3,5-difluorophenyl)-1-methyl-3-[4-(methylsulfonyl)phenyl]propyl}-4-(2-{[4-(methylsulfonyl)phenyl]sulfonyl}ethyl)piperidine

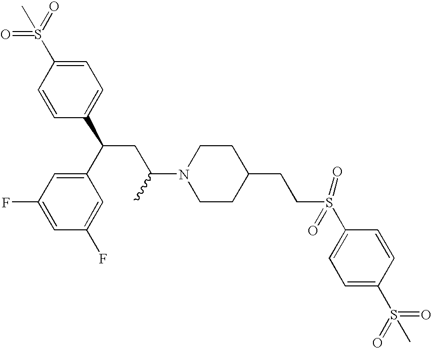

To stirred solution of (4R)-4-(3,5-difluorophenyl)-4-[4-(methylsulfonyl)phenyl]butan-2-one (500 mg) in THF (50 ml) was added 2 equivalents of 4-(2-{[4-(methylsulfonyl)phenyl]sulfonyl}ethyl)piperidine and 5 ml titanium isopropoxide, then stirred for 1 hour at 20-25° C. Sodium tris-acetoxyborohydride (2.5 g) was then added and stirring continued for 16 hours, then 2N NaOH (10 ml) was added and the organic layer decanted from the white precipitate. The inorganic solids were slurried again with THF and the combined organic layers were dried and evaporated. The crude product was subjected to chromatography on silica, eluting with ethyl acetate to give a pure sample of one of the diastereomers (yield 30 mg).

NMR (CDCl₃): 0.91 (d, 3H), 1.1-2.6 (m, 13H), 2.77 (m, 1H), 3.04(s, 3H), 3.12 (s, 3H), 3.16 (m, 2H), 4.25 (m, 1H), 6.66 (t, 1H), 6.76 (d, 2H), 7.40 (d, 2H), 7.86 (d, 2H), 8.12 (d, 2H), 8.18 (d, 2H).

### EXAMPLE 17

### Preparation of the hydrochloride salt of 1-{(3R)-3-(3,5-difluorophenyl)-3-[4-(methylsulfonyl)phenyl]propyl}-4-(2-{[4-(methylsulfonyl)phenyl]sulfonyl}ethyl)piperidine.

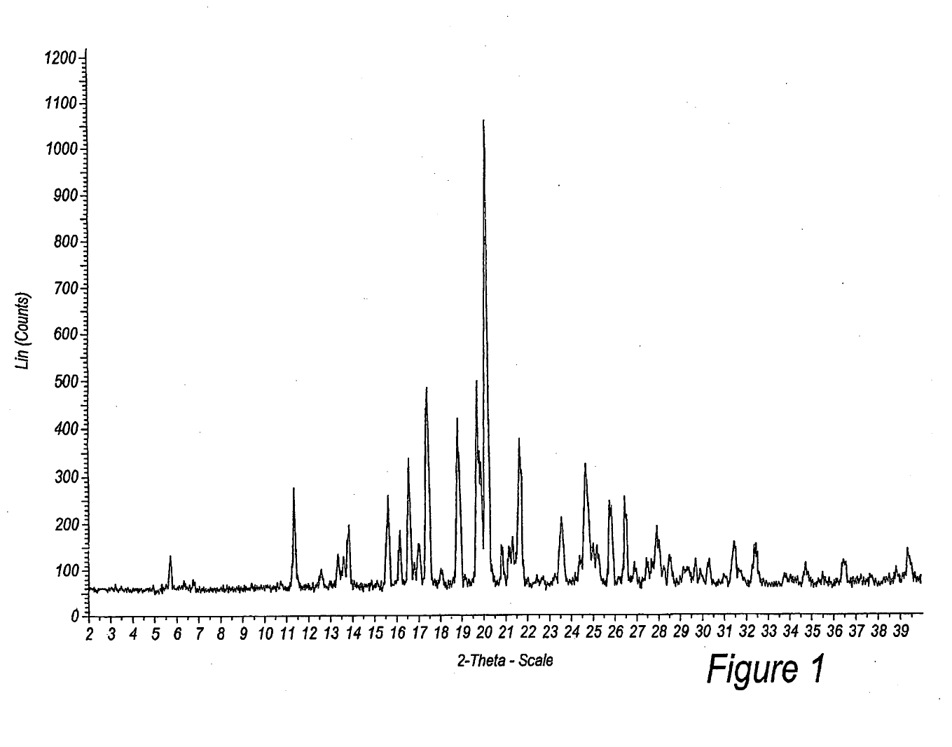

4M HCl in dioxane (0.08 ml) was added to a hot solution of 1-{(3R)-3-(3,5-difluorophenyl)-3-[4-(methylsulfonyl)phenyl]propyl}-4-(2-{[4-(methylsulfonyl)phenyl]-sulfonyl}ethyl)piperidine (0.2 g) in ethanol (25 ml) and the solution was allowed to cool and stand at room temperature for 16 hours. The hydrochloride salt obtained was filtered and dried, yield 197 mg. A sample of the salt obtained was crystallized from ethanol, filtered and dried. PXRD of this compound is presented in FIG. 1.

### EXAMPLE 18

### Preparation of the maleate salt of 1-{(3R)-3-(3,5-difluorophenyl)-3-[4-(methylsulfonyl)phenyl]propyl}-4-(2-{[4-(methylsulfonyl)phenyl]sulfonyl}ethyl)piperidine

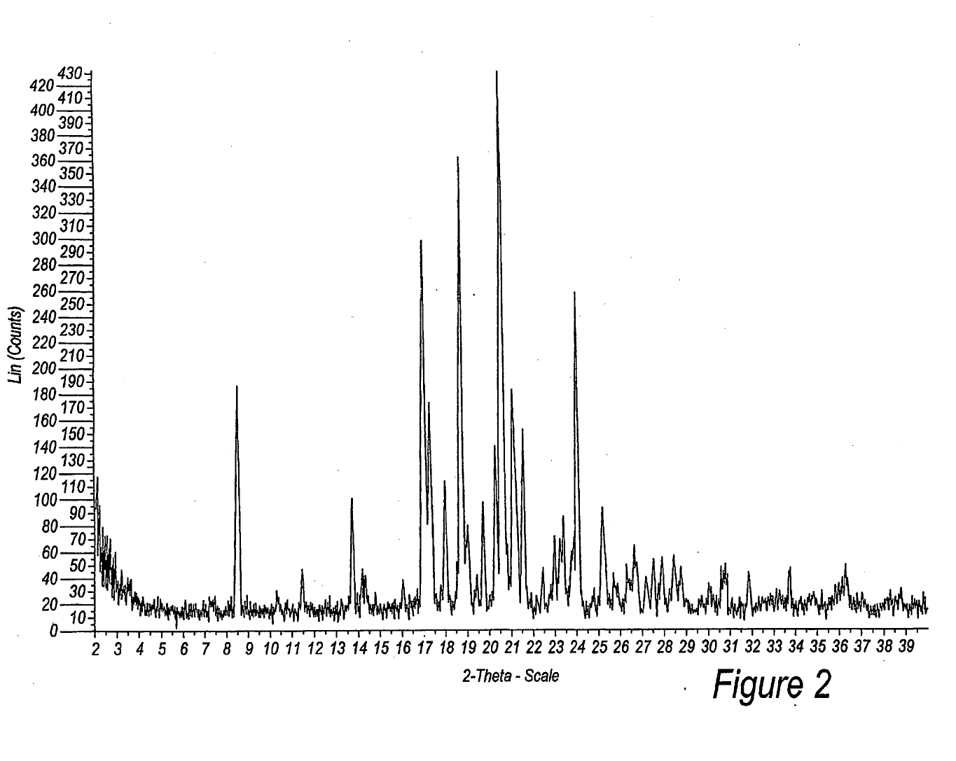

1-{(3R)-3-(3,5-Difluorophenyl)-3-[4-(methylsulfonyl)phenyl]propyl}-4-(2-{[-4-(methylsulfonyl)phenyl]sulfonyl}ethyl)piperidine (3 g) was dissolved in a mixture of ethyl acetate (50 ml) and ethanol (25 ml) at 50° C. Meanwhile maleic acid (0.6 g) was dissolved in ethanol (25 ml) at 50° C. and, when both solutions were ready, the solution of maleic acid was poured into the solution of free base. The resulting mixture was stirred while allowing to cool and then filtered after 1 hour and the residue (title compound) washed with ethyl acetate. The residue was dired in a vacuum oven to leave the title compound (about 3.5 g, approximately 95% yield). PXRD of this maleate salt is presented in FIG. 2.

The succinate, malonate and fumarate salts of 1-{(3R)-3-(3,5-difluorophenyl)-3-[4-(methylsulfonyl)phenyl]propyl}-4-(2-{[4-(methylsulfonyl)phenyl]sulfonyl}ethyl)piperidine were prepared using the method of Example 18. The fumarate salt of 1-{(3R)-3-(3,5-difluorophenyl)-3-[4-(methylsulfonyl)phenyl]propyl}4-(2-{[4-(methylsulfonyl)phenyl]sulfonyl}ethyl)piperidine was formed as a crystalline solid. The tartrate salt was formed as a gum.

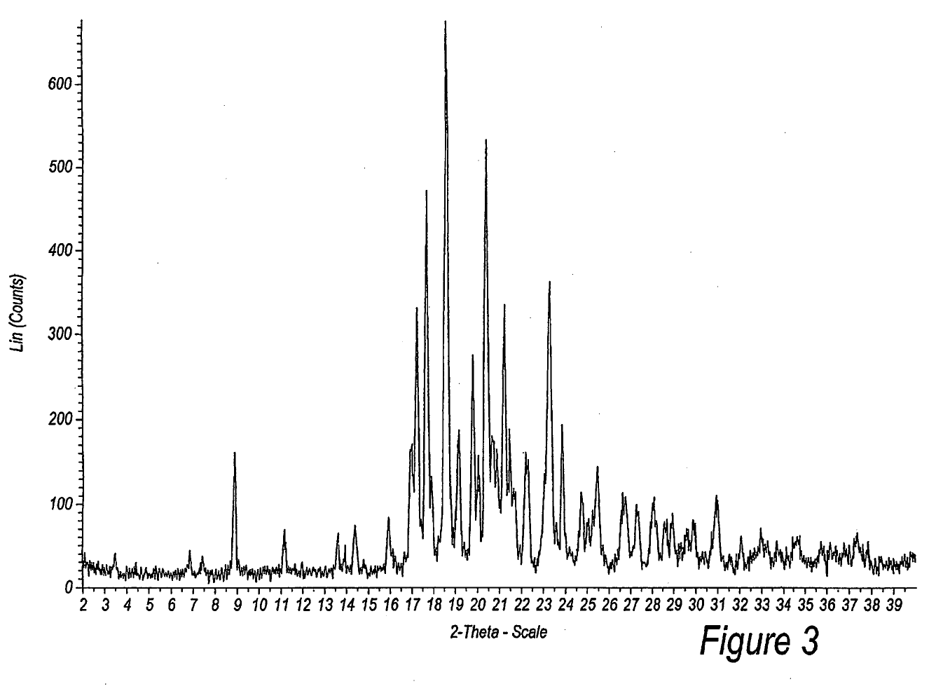

PXRD of the succinate salt of 1-{(3R)-3-(3,5-difluorophenyl)-3-[4-(methylsulfonyl)phenyl]propyl)-4-(2-{[4-(methylsulfonyl)phenyl]sulfonyl}ethyl)piperidine is presented in FIG. 3.

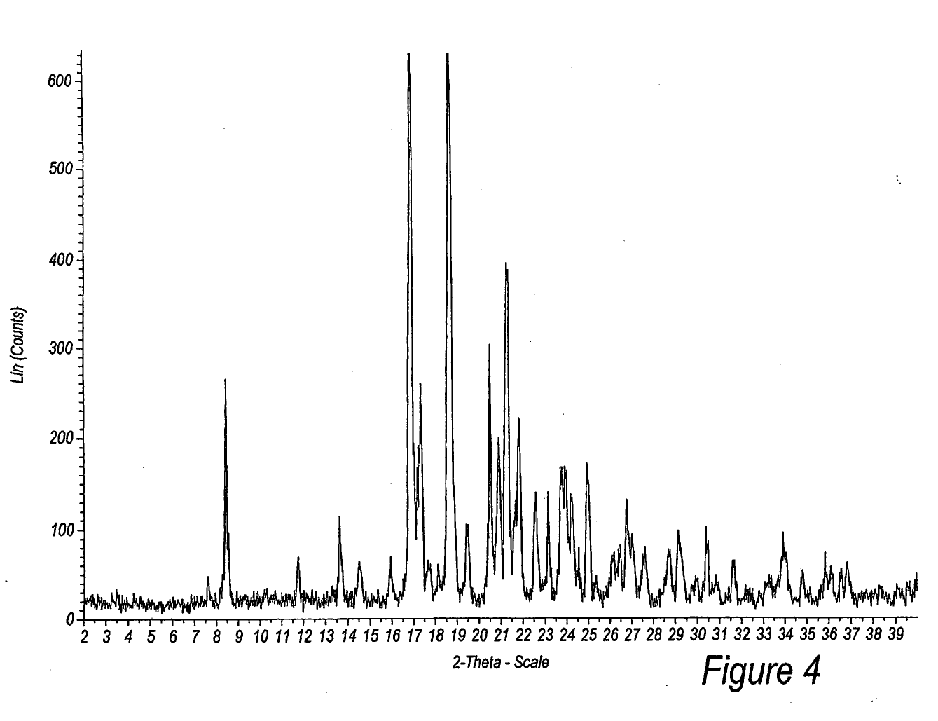

PXRD of the malonate salt of 1-{(3R)-3-(3,5-difluorophenyl)-3-[4-(methylsulfonyl)phenyl]propyl}-4-(2-{[4-(methylsulfonyl)phenyl]sulfonyl}ethyl)piperidine is presented in FIG. 4.

Preparation of certain intermediates is now presented in Methods A to U.

Method A

### (S)-3-Phenyl-3-(4-methanesulfonylphenyl)propionaldehyde

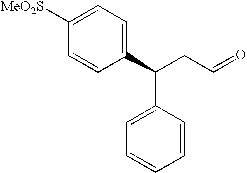

### Step 1: Preparation of (4S, 5R)-1-[(S)-3-(4-methanesulfonyl-phenyl)-3-phenyl-propionyl]-3,4-dimethyl-5-phenyl-imidazolidin-2-one

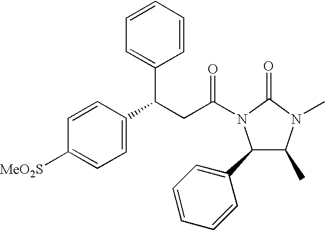

To a mixture of copper (I) iodide (960 mg, 5.0 mmol) and THF (20 mL) was added N,N,N′,N′-tetramethylethylenediamine (0.83 mL, 5.5 mmol) and the resulting mixture was stirred at room temperature for 10 min. then cooled to −78° C. Phenylmagnesium bromide (5.0 mL, 1M in THF, 5.0 mmol) was added and the resulting mixture stirred at −78° C. for 15 min. A solution of di-n-butylboron triflate (3.0 mL, 1M in diethyl ether, 3.0 mmol) and (E)-(4S, 5R)-1-(3-[4-methanesulfonylphenyl]acryloyl)-3,4-dimethyl-S-phenyl-imidazolidin-2-one (step 4 below), 1.0 g, 2.51 mmol) in THF (15 mL) was added and the resulting mixture was stirred whilst allowing to warm to room temperature for 18 h. The reaction mixture was washed with saturated aqueous ammonium chloride, water and brine, dried (MgSO₄) and evaporated. The residue was purified by eluting through a 20 g Bond Elut with gradient of isohexane to ethyl acetate giving the sub-titled compound (1.49 g, 100%); NMR (CDCl₃): 0.78 (d, 3H), 2.82 (s, 3H), 3.00 (s, 3H), 3.78 (dd, 1H), 3.80 (m, 1H), 3.98 (dd, 1H), 4.72 (m, 1H), 5.19 (d, 1H), 6.99 (m, 2H), 7.22 (m, 8H), 7.48 (d, 2H), 7.79 (d, 2H); MS: 477 (MH+).

### Step 2: Preparation of (S)-3-phenyl-3-(4-methanesulphonylphenyl)propan-1-ol

To a solution of (4S, 5R)-1-[(S)-3-(4-methanesulphonyl-phenyl)-3-phenyl-propionyl]-3,4-dimethyl-5-phenyl-imidazolidin-2-one (846 mg, 1.78 mmol) in THF (20 mL) at 0° C. was added lithium aluminium hydride (3.6 mL, 1M in THF, 3.6 mmol) and the resulting mixture was stirred for 15 min. The reaction was quenched by the addition of 2M aqueous sodium hydroxide. The phases were separated and the organic phase pre-absorbed onto a Bond Elut and eluted with a gradient of isohexane to ethyl acetate giving the sub-titled compound as a white solid (285 mg, 55%); NMR (CDCl₃): 1.63 (br s, 1H), 2.33 (m, 2H), 3.00 (s, 3H), 3.59 (t, 2H), 4.28 (t, 1H), 7.23 (m, 5H), 7.43 (d, 2H), 7.82 (d, 2H).

### Step 3: Preparation of the Title Compound

To a solution of (s)-3-phenyl-3-(4-methanesulfonylphenyl)propan-1-ol (244 mg, 0.84 mmol) in DCM (5 mL) was added Dess-Martin periodinane (392 mg, 0.92 mmol) and the resulting mixture was stirred at room temperature for 1.5 h. The mixture was washed with 2M aqueous sodium hydroxide (2×10 mL), dried and evaporated to give the title compound.

### Step 4: Preparation of E-(4S, 5R)-1-(3-[4-Methanesulphonylphenyl]acryloyl)-3,4-dimethyl-5-phenyl-imidazolidin-2-one

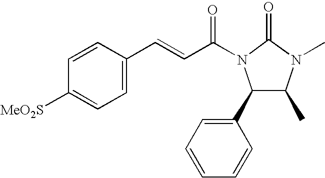

To a stirred solution of 3-(4-methanesulphonylphenyl)acrylic acid (7.14 g, 31.5 mmol) in DCM (10 mL) was added thionyl chloride (3 mL, 34.7 mmol) dropwise and the resulting mixture was stirred at room temperature for 18 h. To this solution was added DIPEA (5.04 mL, 28.9 mmol) dropwise at room temperature. The resulting solution was added to a stirred solution of (4R, 5S)-1,5-dimethyl-4-phenyl-imidazolidin-2-one (5.0 g, 26.3 mmol) in DCM (20 mL) and DIPEA (4.58 mL, 26.9 mmol) and the resulting mixture stirred at room temperature for 4 h. The mixture was washed with water and brine, pre-absorbed onto a Bond Elut and eluted with a gradient of isohexane to ethyl acetate giving the title compound as a solid (7.61 g, 73%); NMR (CDCl₃): 0.84 (d, 3H), 2.89 (s, 3H), 3.04 (s, 3H), 3.98 (m, 1H), 5.42 (d, 1H), 7.20 (m, 2H), 7.32 (m, 3H), 7.69 (d, 1H), 7.74 (d, 2H), 7.93 (d, 2H), 8.31 (d, 1H); MS: 399 (MH+).

Method B

### 4-[2-(4-Methanesulphonylphenylsulphonyl)ethyl]piperidine

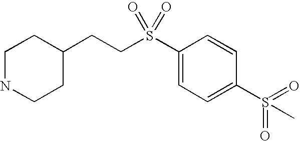

### Step 1 Preparation of N-tert-butoxycarbonyl-4-[2-(4-methylthiophenylthio)ethyl]piperidine

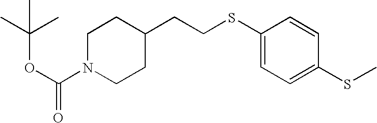

4-Methylthiobenzenethiol (1.16 g) was added to a suspension of sodium hydride (297 mg of 60% dispersion in mineral oil) in DMF (20ml) at 0° C. and stirred at this temperature for 30 minutes. N-tert-Butoxycarbonyl-4-[2-(4-toluenesulphonyloxy)ethyl]-piperidine (CAS No. 89151-45-1) (2.84 g) was added, the reaction mixture was allowed to warm to room temperature and was stirred for 16 hours. The reaction mixture was evaporated to dryness and the residue obtained was dissolved in dichloromethane (30 ml) and the solution was washed with water (20 ml) and brine (20 ml) and dried. The residue obtained on removal of the solvent was chromatographed on a 50 g silica Bond Elut column eluting with a solvent gradient of isohexane—50% ethyl acetate/isohexane. Yield 2.5 g, MH⁺ 268.

### Step 2 Preparation of N-tert-butoxycarbonyl-4-[2-(4-methylsulphonylphenylsulphonyl)ethyl]-piperidine

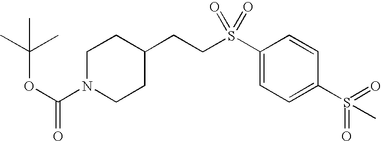

m-Chloroperbenzoic acid (5.64 g) was added to a solution of N-tert-butoxycarbonyl-4-[2-(4-methylthiophenylthio)ethyl]piperidine (2.1 g) in dichloromethane (90 ml) at 0° C. The reaction mixture was allowed to warm to room temperature and was stirred for 16 hours. The reaction mixture was washed with saturated aqueous sodium bicarbonate solution (20 ml), water (20 ml) and brine (20 ml) then dried and evaporated to dryness. The product was chromatographed on a 50 g silica Bond Elut column eluting with a solvent gradient of 20% ethyl acetate/isohexane—ethyl acetate to give the product, yield 1.82 g, MH⁺ 375.9.

### Step 3 Preparation of Title Compound

Variant A

Trifluoroacetic acid (5 ml) was added to a solution of N-tert-butoxycarbonyl-4-[2-(4-methylsulphonylphenylsulphonyl)ethyl]piperidine (1.94 g) in dichloromethane (20 ml) and was allowed to stand at room temperature for 1 hour. The reaction mixture was evaporated to dryness and the residue was dissolved in 2M sodium hydroxide (15 ml) and extracted with dichloromethane (3×20 ml). The combined dichloromethane extracts were dried and evaporated to dryness to give the title compound, yield 1.3 g, MH⁺331.9.

Variant B

A solution of 4M hydrochloric acid in dioxane (15 ml) was added to a stirred solution of N-tert-butoxycarbonyl-4-[2-(4-methylsulphonylphenylsulphonylethyl]piperidine (5.62 g) in dichloromethane (15 ml) and stiring was continued for 1 hour. The reaction mixture was triturated with diethyl ether and the solid formed was filtered, washed with diethyl ether and dried under high vacuum. The title compound was obtained as its hydrochloride salt, yield 4.88 g, M⁺H 331.9.

The following compounds were also prepared using a process analogous to Method B

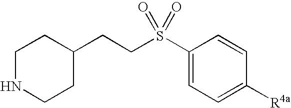

| R^{4a} | MH⁺ |
|---|---|
| Cyano | 279 |
| Fluoro | 272 |
| Methyl | 268 |
| Methoxy | 284 |
| Hydroxy | 270 |

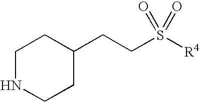

| R⁴ | MH⁺ |
|---|---|
| methyl | 192 |
| cyclohexyl | 260 |
| pyridin-3-yl | 255 |
| 1-methyl-imidazol-2-yl | 258 |
| 5-methyl-1,3,4-thiadiazol-2-yl | 276 |

Method C

### 3-Phenyl-3-(N-methanesulphonylpiperidin-4-yl)propionaldehyde

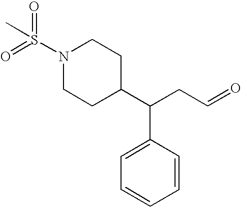

### Step 1 Preparation of 4-benzoyl-1-methanesulphonylpiperidine

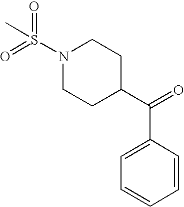

Methanesulphonyl chloride was added to a stirred slurry of 4-benzoylpiperidine hydrochloride (4.51 g) and triethylamine (8.35 ml) in dichloromethane (100 ml) at 0° C. The reaction mixture was allowed to warm to room temperature and was stirred for 16 hours. The mixture was diluted with dichloromethane (50 ml) and washed with ammonium chloride solution (2×25 ml) and brine (25 ml), dried and evaporated to dryness to give 4-benzoyl-1-methanesulphonylpiperidine as a white solid, yield 3.98 g. NMR (CDCl₃): 1.93 (m, 4H), 2.81 (s, 3H), 2.98 (d-t, 2H), 3.40 (m, 1H), 3.77 (m, 2H), 7.43 (t, 2H), 7.57 (t, 1H), 7.89 (d, 2H).

### Step 2 Preparation of ethyl 3-phenyl-3-(N-methanesulphonylpiperidin-4-yl)acrylate

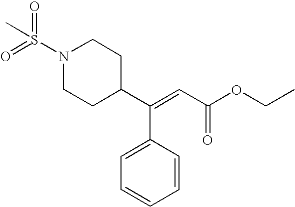

Lithium bis(trimethylsilyl)amide (16.3 ml of a 1M solution in THF) was added dropwise to a solution of triethylphosphonoacetate (2.93 ml) in THF at 0° C. under an argon atmosphere and the mixture was stirred for 30 minutes. A slurry of 4-benzoyl-1-methanesulphonylpiperidine (3.96 g) in THF (30 ml) was added, the reaction mixture was allowed to warm to room temperature and stirring was continued for 24 hours. The reaction mixture was diluted with dichloromethane (80 ml) and water (80 ml). The organic layer was washed with water and the combined aqueous extracts were in turn extracted with dichloromethane (50 ml). The combined dichloromethane extracts were washed with brine (25 ml), dried and evaporated to dryness. The residue was chromatographed on a 90 g Biotage column eluted with a solvent gradient (30-5% ethyl acetate/isohexane to give a less polar fraction (1.62 g) and a more polar fraction (0.53 g). Both fractions (cis/trans isomers) were combined and used for the next step.

Less polar NMR (CDCl₃): 1.27 (t, 3H), 1.69 (m, 2H), 1.81 (d, 2H), 2.72 (s, 3H), 2.72 (t, 2H), 3.81 (d, 2H), 3.88 (m, 1H), 4.21 (q, 2H), 5.78 (s, 1H), 7.11 (m, 2H), 7.27 (m, 3H).

More polar NMR (CDCl₃): 1.01 (t, 3H), 1.56 (m, 2H), 1.85 (d, 2H), 2.31 (m, 1H), 2.63 (t, 2H), 2.74 (s, 3H), 3.83 (d, 2H), 3.92 (q, 3H), 5.82 (s, 1H), 7.04 (d, 2H), 7.30 (m, 3H).

### Step 3 Preparation of ethyl 3-phenyl-3-(N-methanesulphonylpiperidin-4-yl)propionate

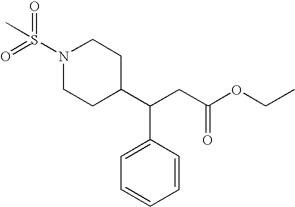

A solution of ethyl 3-phenyl-3-(N-methanesulphonylpiperidin-4-yl)acrylate (2.06 g) in ethanol (30 ml) was hydrogenated over 24 hours under a hydrogen filled balloon using 20% palladium hydroxide as catalyst. The reaction mixture was filtered through Celite and the filtrate evaporated to dryness. The product obtained was used for the next step without further purification. MH⁺ 340.

### Step 4 3-Phenyl-3-(N-methanesulphonylpiperidin-4-yl)propan-1-ol

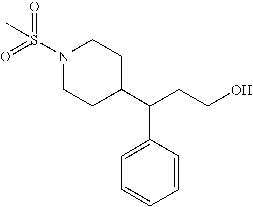

A solution of ethyl 3-phenyl-3-(N-methanesulphonylpiperidin-4-yl)propionate (2 g) in THF (10 ml) was added to a suspension of lithium aluminium hydride (232 mg) in THF (20 ml) at 0° C. under argon over 30 minutes. The reaction mixture was allowed to warm to room temperature and stirred for 2 hours. Water (10 ml) was added followed by magnesium sulphate (10 g). The reaction mixture was filtered and the filtrate evaporated to dryness to give the product as a white foam, yield 1.57 g. NMR (CDCl₃): 1.40 (m, 4H), 1.57 (m, 1H), 1.78 (m, 1H), 2.01 (m, 2H), 2.45 (m, 2H), 2.58 (t, 1H), 2.70 (m, 3H), 3.31 (m, 1H), 3.42 (m, 1H), 3.67 (d, 1H), 3.80 (d, 1H), 7.04 (d, 1H), 7.19 (t, 1H), 7.29 (q, 2H).

### Step S Preparation of the Title Compound

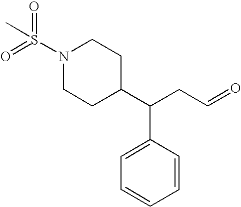

Dess-Martin periodinane (739 mg) was added to a stirred solution of 3-phenyl-3-(N-methanesulphonylpiperidin-4-yl)propan-1-ol (454 mg) in dichloromethane (8 ml) and stirring was continued for 2 hours. The reaction mixture was diluted with dichloromethane (100 ml) and washed with 2M sodium hydroxide (2×50 ml), brine (50 ml) and dried. The product obtained on removal of the solvent was used in subsequent steps without purification.

Method D

### N-(3-Phenyl-3-chloropropyl)-4-[2-(4-methanesulphonylphenylsulphonyl)ethyl]-piperidine

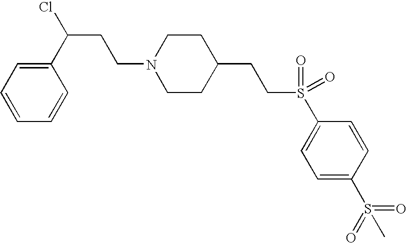

Triethylamine (0.73 ml) was added to a solution of N-(3-hydroxy-3-phenylpropyl)-4-[2-(4-methanesulphonylphenylsulphonyl)ethyl]-piperidine (1.22 g) in dichloromethane (20 ml) followed by methanesulphonyl chloride (0.33 g) and the mixture was stirred for 18 hours at room temperature. The reaction mixture was washed successively with water (25 ml) and brine (25 ml) and dried. The residue obtained after removal of the solvent was chromatographed on a 20 g silica Bond Elut column eluted with a solvent gradient of ethyl acetate—20% methanol/ethyl acetate to give the title compound, yield 0.73 g, H 483.99. NMR(CDCl₃): 1.3 (m, 3H), 1.6 (m, 4H), 1.9 (m, 2H), 2.1-2.3 (m, 2H), 2.4 (m, 2H), 2.8-2.9 (m, 2H), 3.1 (s, 3H), 3.2 (m, 2H), 5.0 (m, 1H), 7.3 (m, 5H), 8.2 (m, 4H).

### N-(3-Hydroxy-3-phenylpropyl)-4-[2-(4-methanesulphonylphenylsulphonyl)ethyl]-piperidine

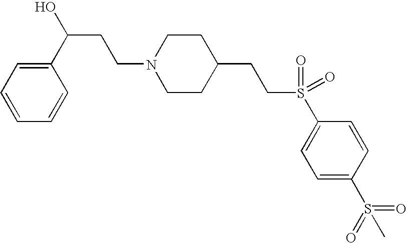

Sodium borohydride (100 mg) was added in portions to a solution of N-(3-oxo-3-phenylpropyl)-4-[2-(4-methanesulphonylphenylsulphonyl)ethyl]-piperidine (1.22 g) in ethanol (20 ml) at room temperature and was stirred for 18 hours. The reaction mixture was evaporated to dryness and the residue was dissolved in dichloromethane (30 ml) and this solution was washed with water (25 ml), brine (25 ml) and dried. Removal of the solvent gave the title compound as white solid, yield 1.21 g, MH⁺ 465.98.

### N-(3-Oxo-3-phenylpropyl)-4-[2-(4-methanesulphonylphenylsulphonyl)ethyl]-piperidine

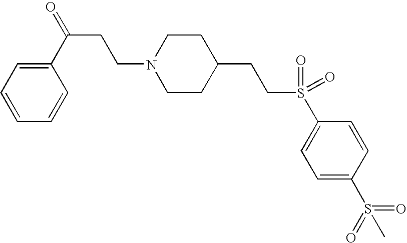

3-Chloropropiophenone (0.726 g) was added to a mixture of 4-[2-(4-methanesulphonylphenylsulphonyl)ethyl]-piperidine (1.3 g) (prepared as described in Method B) and potassium carbonate (1.09 g) in DMF (20 ml) and the mixture was stirred at room temperature for 1 hour. The reaction mixture was evaporated to dryness and the residue was dissolved in dichloromethane (30 ml). The dichloromethane solution was washed with water (25 ml), brine (25 ml) and dried. The residue obtained after removal of the solvent was chromatographed on a 50 g silica Bond Elut column eluted with a solvent gradient of ethyl acetate—20% methanol/ethyl acetate to give the title compound as a white solid, yield 1.22 g, MH⁺ 463.97. NMR(CDCl₃): 1.2-1.4 (m, 3H), 1.6 (m, 4H), 2.0 (m, 2H), 2.8 (m, 2H), 2.9 (m, 2H), 3.1-3.2 (m, 7H), 7.4 (m, 2H), 7.5(m, 1H), 7.9 (m, 2H), 8.2 (m, 4H).

Method E

### (R)-3-(3,5-Difluorophenyl)-3-(4-methanesulfonylphenyl)propionaldehdye

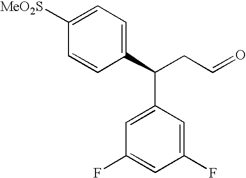

This was prepared from (4S, 5R)-1-(3-[4-methanesulfonylphenyl]acryloyl)-3,4-dimethyl-5-phenyl-imidazolidin-2-one and 3,5-difluorophenylmagnesium bromide using a method similar to that used to prepare (S)-3-phenyl-3-(4-methanesulfonyl-phenyl)propionaldehyde from phenylmagnesium bromide (Method A); NMR (CDCl₃): 3.05 (s, 3H), 3.20 (d, 2H), 4.72 (t, 1H), 6.75 (m, 3H), 7.35 (d, 2H), 7.88 (d, 2H), 9.75 (s, 1H).

Method F

### (S) N-(3-hydroxy-3-phenylpropyl)-4-(2-(4-fluorophenylsulphonyl)ethyl]-piperidine

### Step 1: (R or S) N-(3-chloro-3-phenylpropyl)-4-[2-(4-fluorophenylsulphonyl)ethyl]-piperidine

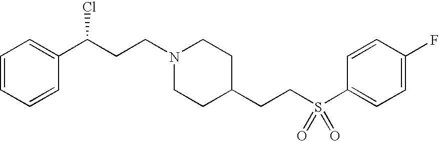

Methanesulphonyl chloride (158 μl) was added to a solution of (S) N-(3-hydroxy-3-phenylpropyl)-4-[2-(4-fluorophenylsulphonyl)ethyl]-piperidine (600 mg) and triethylamine (417 μl) in dichloromethane (10 ml) maintained at 0° C. under an argon atmosphere. The reaction mixture was allowed to warm to room temperature and was stirred for 18 hours. The reaction mixture was diluted with dichloromethane (50 ml) and washed with saturated amonium chloride solution (2×25 ml) and brine (25 ml) and dried. Removal of the solvent gave the title compound which was used without further purification. NMR: (CDCl₃): 1.18-2.24 (m, 13H), 2.78 (m, 1H), 2.84 (m, 1H), 3.04 (1H, m), 4.92 (t, 1H), 7.20-7.40 (m, 7H), 7.91 (m, 2H); MS 424(MH⁺).

### Step 2: (S) N-(3-hydroxy-3-phenylpropyl)-4-[2-(4-fluorophenylsulphonyl)ethyl]-piperidine

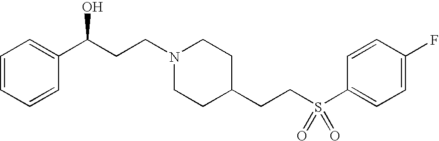

A solution of (S)-1-phenyl-3-(4-toluenesulphonyl)oxypropan-1-ol (459 mg) in dioxane (10 ml) was added to a suspension of 4-[2-(4-fluorophenylsulphonyl)ethyl]piperidine (407 mg) and potassium carbonate (414 mg) and the mixture was heated at 95° C. for 17 hours. The reaction mixture was allowed to cool and was partitioned between dichloromethane (100 ml) and water (50 ml). The organic layer was collected and washed with water (50 ml), brine (50 ml) and dried. Removal of the solvent gave the title compound, yield 607 mg. NMR: (CDCl₃): 1.18-1.69 (m, 8H), 1.82 (m, 3H), 2.02 (m, 1H), 2.48 (m, 1H), 2.62 (m, 1H), 2.93 (d, 1H), 3.05 (m, 3H), 4.89 (m, 1H), 7.21-7.40 (m, 7H), 7.92 (m, 2H); MS 406 (MH⁺).

Method G

### (R) 3-(1-Methanesulphonylpiperidinyl)-3-[3,5-difluorophenyl]propionaldehyde

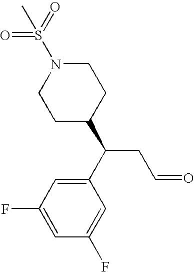

### Step 1 3-[N-(Benzyloxycarbonylpiperidin-4-yl)]propenoic acid

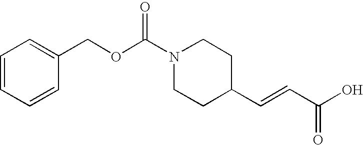

A mixture of N-benzyloxycarbonyl-4-formylpiperidine (log), malonic acid (4.2), pyridine (4 ml) and piperidine (0.4 ml) was heated at 100° C. for 2 hours. The reaction mixture was allowed to cool and was diluted with ethyl acetate (100 ml). The solution was washed with 2M HCl (2×100 ml), dried and evaporated to dryness. The residue was triturated with isohexane to give the title compound, yield 13.5 g.

NMR (DMSOd6): 1.2 (m, 2H), 1.7 (m, 2H), 2.35 (m, 1H), 2.85 (m, 2H), 4 (d, 2H), 5.05 (s, 2H), 5.75 (d, 1H), 6.75 (m, 1H), 7.35 (m, 5H), 12.25 (broad peak, 1H).

### Step 2 N-(Benzyloxycarbonylpiperidin-4-yl)propenoic acid isopropyl ester

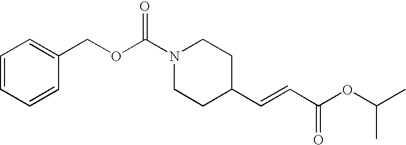

A solution of N-(benzyloxycarbonylpiperidin-4-yl)propenoic acid (52 g) in isopropanol (500 ml) containing concentrated sulphuric acid (20 ml) was heated under reflux for 32 hours. The solvent was evaporated and the residue was dissolved in ethyl acetate (250 ml). The ethyl acetate solution was washed with water (2×250 ml) and saturated aqueous sodium bicarbonate (2×25 ml) and dried. The residue obtained on evaporation of the solvent. was chromatographed on a Bond Elut cartridge eluted with a solvent gradient (isohexane—25% ethyl acetate/isohexane) to give the title compound, yield 54 g.

### Step 3 Preparation of (R) 3-(N-benzyloxycarbonylpiperidin-4yl)-3-(3,5-difluorophenyl)propanoic acid isopropyl ester

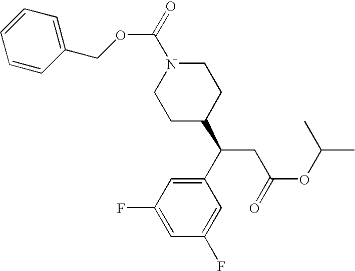

Dioxane (100 ml) was charged to a 500 ml three necked flask and purged with argon for 10 minutes. Acetylacetonatobis[ethylene]rhodium (I) (620 mg) and R-BINAP were added and the mixture was stirred for 10 minutes. 3,5-Difluorophenylboronic acid (19 g) was added and the mixture was purged with argon for 10 minutes. N-(benzyloxycarbonylpiperidin-4-yl)propenoic acid isopropyl ester (8 g) and ethanediol (20 ml) in dioxane (100 ml) were added and the mixture was purged with argon for 10 minutes. The mixture was heated at 100° C. for 18 hours, allowed to cool and was passed through activated alumina (200 g) washed through with ethyl acetate (3×100 ml). The combined washings were evaporated to dryness and the residue obtained was dissolved in ethyl acetate (100 ml) and washed successively with saturated aqueous sodium bicarbonate (2×100 ml) and 2M HCl (2×100 ml), dried and evaporated to dryness. The product obtained (12 g) was shown to be 40% of the required material by NMR and was used without further purification in the subsequent reactions.

### Step 4 Preparation of (R) 3-(piperidin-4-yl)-3-(3,5-difluorophenyl)propanoic acid isopropyl ester

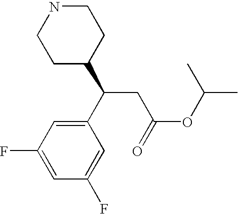

A solution of (R) 3-(N-benzyloxycarbonylpiperidin-4-yl)-3-(3,5-difluorophenyl)propanoic acid isopropyl ester (12 g) in ethanol (300 ml) containing 20% palladium hydroxide on charcoal (2 g) was hydrogenated under a hydrogen filled balloon. The catalyst was filtered and the filtrate was evaporated to dryness to give the title compound (10 g) which was used without further purification.

### Step 5 Preparation of (R) 3-(N-methanesulphonylpiperidin-4-yl)-3-(3,5-difluorophenyl)-propanoic acid isopropyl ester

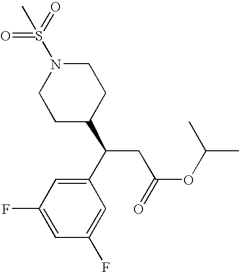

Methanesulphonyl chloride (3.7 g) was added to a solution of (R) 3-(piperidin-4-yl)-3-(3,5-difluorophenyl)propanoic acid isopropyl ester (log) and triethylamine (3.89 g) in dichloromethane (100 ml) at 0° C. The reaction mixture was allowed to warm to room temperature and was washed with 2M HCl (2×50 ml) and saturated aqueous sodium bicarbonate (2×50 ml), dried and evaporated to dryness to give the title compound (10 g) which was used without further purification.

### Step 6 Preparation of (R) 3-(N-methanesulphonylpiperidin-4-yl)-3-(3,5-difluorophenyl)propanol

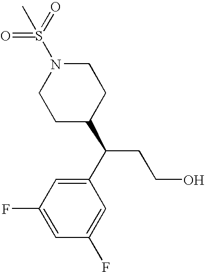

Lithium aluminium hydride (25 ml of a 1M solution in THF) was added dropwise over 15 minutes to a solution of (R) 3-(N-methanesulphonylpiperidin-4-yl)-3-(3,5-difluorophenyl)propanoic acid isopropyl ester (10 g) in THF (150 ml) at −10° C. The reaction mixture was stirred at −10° C. for 30 minutes, 2M NaOH (25 ml) was added, the mixture was filtered and the filtrate evaporated to dryness. The residue obtained was dissolved in ethyl acetate and washed with 2M HCl (2×100 ml) and dried. The residue obtained on removal of the solvent was chromatographed on a Bond Elut column eluting with a solvent gradient (80% ethyl acetate/isohexane—ethyl acetate) to give the title compound, yield 2.2 g.

NMR (DMSO d6): 0.95-1.2 (m, 2H), 1.3 (m, 1H), 1.6 (m.2H), 1.9 (m, 2H), 2.6 (m, 2H), 2.8 (s, 3H), 3.1 (m, 1H), 3.2 (m, 1H), 3.4 (m, 1H), 3.5 (m, 1H), 6.8-7 (m, 3).

### Step 7 Preparation of (R) 3-(N-methanesulphonylpiperidin-4-yl)-3-(3,5-difluorophenyl)propionaldehyde

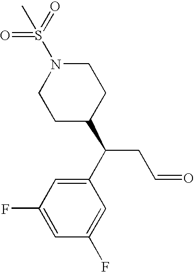

Dess-Martin periodinane (1 g) was added to a solution of (R) 3-(N-methanesulphonylpiperidin-4-yl)-3-(3,5-difluorophenyl)propanol (0.8 g) in dichloromethane (40 ml) and the mixture was stirred for 1.5 hours. The reaction mixture was washed with 2M NaOH (2×20 ml) and dried. The solution of the title compound in dichloromethane was used in subsequent reactions.

Method H

### (R) 3-(N-Methanesulphonylpiperidin-4-yl)-3-phenylpropanol

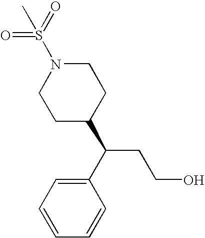

### Step 1 Preparation of 3-(N-methanesulphonylpiperidin-4-yl)propenoic acid acid chloride

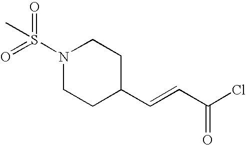

1-Chloro-N,N,2-trimethylpropenylamine (1.06 ml) was added dropwise over 10 minutes to a suspension of 3-(N-methanesulphonylpiperidin-4-yl)propenoic acid (1.86 g, prepared from N-methanesulphonylpiperidine-4-carboxaldehyde [CAS 241134-35-0] according to step 1 of Method E) in THF (20 ml) under an atmosphere of argon and the mixture was stirred for 2 hours and used directly in step 2.

### Step 2 Preparation of 1-[3-(N-methanesulphonylpiperidin-4-yl)propenyl]-(4S, 5R)-3,4-dimethyl-4-phenyl-imidazolidin-2-one

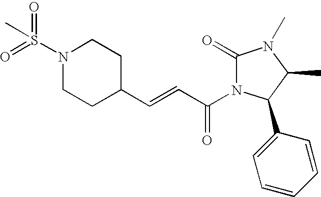

Lithium bis(trimethylsilyl)amide (8 ml of a 1M solution in THF) was added dropwise to a suspension of (4R,5S)-1,5-dimethyl-4-phenyl-2-imidazolidinone (1.52 g) in THF (20 ml) under argon at -10° C. The reaction mixture was stirred at −10° C. for 10 minutes, allowed to warm to 0° C. and maintained at this temperature for 10 minutes then cooled again to −10° C. The solution of the acid chloride prepared in Step 1 was added dropwise and the reaction mixture was allowed to warm to room temperature and washed with water (100 ml). The aqueous extract was extracted with ethyl acetate (3×50 ml) and the ethyl acetate extracts were dried and the residue passed through a 90 g Biotage column eluting with a solvent gradient (50% ethyl acetate/isohexane—70% ethyl acetate/isohexane). Yield 1.89 g. LC-MS MH⁺ 406, NMR (CDCl₃): 0.8 (d, 3H), 1.5-1.6 (m, 3H), 1.9 (m, 2H), 2.3 (m, 1H), 2.7 (m, 2H), 2.75 (s, 3H), 2.8 (s, 3H), 3.75 (m, 2H), 3.9 (m, 1H), 5.3 (d, 1H), 6.85 (d-d, 1H), 7.1 (d, 1H), 7.2-7.35 (m, 3H), 7.45 (d, 1H).

### Step 3 Preparation of (R) 1-[3-phenyl-3-(methanesulphonylpiperidin-4-yl)propionyl]-(4S,5R)-3,4-dimethyl-5-phenyl-imidazolidin-2-one

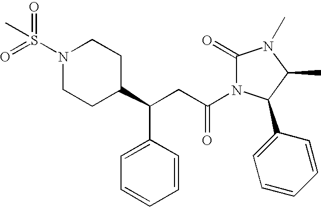

A mixture of copper(I) iodide (1.78 g) and N,N,N′,N′-tetramethylethylenediamine (1.41 ml) in THF (50 ml) was stirred under argon for 1 hour then cooled to −78° C. and phenylmagnesium bromide (5.4 ml of a 1M solution in THF) was added and the mixture was stirred at −78° C. for 30 minutes. A solution of 1-[3-(N-methanesulphonylpiperidin-4-yl)propenyl]-(4S, 5R)-3,4-dimethyl-5-phenyl-imidazolidin-2-one (1.89 g) and dibutylboron triflate (4.67 ml of a 1M solution in diethylether in THF (50 ml) was added over 10 minutes and the reaction mixture was stirred at −78° C. for 1 hour then allowed to warm to room temperature. The reaction mixture was concentrated and filtered through a pad of silica (50 g) washed with ethyl acetate (2×50 ml) and the ethyl acetate washings were washed with 2M HCl (2×150 ml) and dried. The residue obtained on removal of the solvent was passed through a 90 g Biotage column eluting with a solvent gradient (50% ethyl acetate/isohexane—70% ethyl acetate/isohexane) to give the product as a yellow solid, yield 1.34 g, MH⁺ 484. NMR (CDCl₃): 0.7 (d, 3H), 1.2 (m, 1H), 1.35 (m, 1H), 1.5 (m, 1H), 1.9 (m, 1H), 2.45 (m, 1H), 2.55 (m, 1H), 2.7 (s, 3H), 2.8 (s, 3H), 3.1 (m, 1H), 3.2 (d-d, 1H), 3.4 (m, 1H), 3.65 (m, 1H), 3.75-3.9 (m, 3H), 5.2 (d, 1H), 6.7 (d, 2H), 7.05-7.25 (m, 8H).

### Step 4 Preparation of the Title Compound

A solution of (R) 1-[3-phenyl-3-(methanesulphonylpiperidin-4-yl)propionyl]-(4S,5R)-3,4-dimethyl-5-phenyl-imidazolidin-2-one (1.34 g) in THF (14 ml) was added to a solution of lithium aluminium hydride (2.77 ml of a 1M solution in THF) in THF (10 ml) at 0° C. and the mixture was allowed to warm to room temperature over 1 hour. Water (5 ml) was added cautiously, then THF (15 ml) and solid magnesium sulphate. The reaction mixture was filtered and the filtrate was passed through a 40 g Biotage column eluted with a solvent gradient (50% ethyl acetate/isohexane—70% ethyl acetate/isohexane) to give the title compound as a white solid, yield 338 mg. NMR (CDCl₃): 1.15-1.25 (m, 2H), 1.3-1.5 (m, 2H), 1.6 (m, 1H), 1.75 (m, 1H), 1.95-2.10 (m, 2H), 2.5 (m, 2H), 2.6 (m, 1H), 2.7 (s, 3H), 3.3-3.4 (m, 2H), 3.45 (m, 1H), 3.7 (m, 1H), 3.85 (m, 1H), 7.05 (m, 2H), 7.15-7.35 (m, 3H).

Method I

### Preparation of [(piperidin-4-yl)methyl]-(4-methoxyphenylmethyl)sulphone

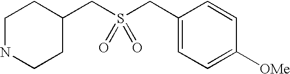

### Step 1 Preparation of [(N-Boc-piperidin-4-yl)methyl]-(4-methoxyphenylmethyl)sulphide

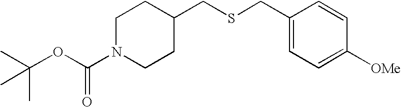

4-Methoxybenzylthiol (0.944 ml) was added to a suspension of sodium hydride (271 mg of a 60% dispersion in mineral oil) in DMF at 0° C. and was stirred at this temperature for 15 minutes. 4-Tosyloxymethyl-N-Boc-piperidine (CAS 166815-96-9) was added and the mixture was allowed to warm to room temperature and was stirred for 3 hours. The reaction mixture was evaporated to dryness and the residue was dissolved in dichloromethane (50 ml) and this solution was washed with water (30 ml) and brine (30 ml) and dried. The solvent was evaporated and the residue was chromatographed on a 50 g Bond Elut column eluting with a solvent gradient (isohexane—20% ethyl acetate/isohexane). Yield 2 g, MH⁺ 252.14. NMR (CDCl₃): 1.0-1.2 (m, 2), 1.4 (s, 9H), 1.5 (m, 1H), 1.7-1.8 (m, 2H), 2.3 (m, 2H), 2.6 (bt, 2H), 3.7 (s, 2H), 3.8 (s, 3H), 4.1 (m, 2H), 6.8 (m, 2H), 7.2 (m, 2H).

### Step 2 Preparation of [(N-Boc-piperidin-4-yl)methyl]-(4-methoxyphenylmethyl)sulphone

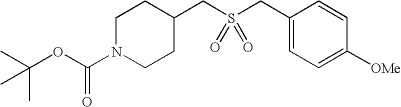

m-Chloroperbezoic acid (2.81 g) was added to a solution of [(N-Boc-piperidin-4-yl)methyl]-(4-methoxyphenylmethyl)sulphide (2 g) in dichloromethane (50 ml) at 0° C. The reaction mixture was allowed to warm to room temperature and was stirred for 16 hours. The reaction mixture was washed with 2M NaOH (20 ml), brine (20 ml) and dried. The solvent was evaporated and the residue was purified on a 50 g silica Bond Elut eluting with a solvent gradient (isohexane—50% ethyl acetate/isohexane) to give the title compouqnd, yield 1.75 g, MH⁺ (−Boc) 284.11.

### Step 3 Preparation of [(piperidin-4-yl)methyl]-(4-methoxyphenylmethyl)sulphone hydrochloride

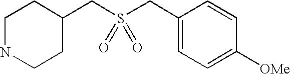

[(N-Boc-piperidin-4-yl)methyl]-(4-methoxyphenylmethyl)sulphone (1.75 g) was stirred with 4M HCl in dioxane (10 ml) for 30 minutes. The reaction mixture was triturated with diethyl ether and the solid obtained was filtered and dried. Yield 1.42 g. MH⁺ 284.

The following compounds were also prepared using a process analogous to Method I

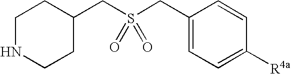

| R^{4a} | MH⁺ |
|---|---|
| Hydrogen | 254 |
| Fluoro | 272 |
| Methyl | 268 |

Method J

### Preparation of [(piperidin-4-yl)methyl]-(4-methanesulphonylphenylmethyl)sulphone

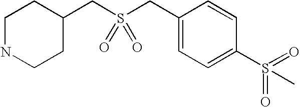

### Step 1 Preparation of [(N-Boc-piperidin-4-yl)methylthioacetate

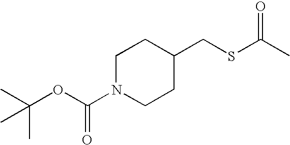

Potassium thioacetate (1.857 g) was added to a solution of 4-tosyloxymethyl-N-Boc-piperidine (CAS 166815-96-9) (3 g) in DMF (40 ml) and the mixture was heated at 100° C. for 4 hours. The reaction mixture was allowed to cool to room temperature and water (5 ml) was added. The reaction mixture was extracted with diethyl ether (3×50 ml). The diethyl ether extracts were washed with saturated aqueous sodium bicarbonate (50 ml), brine (50 ml) and were dried. Removal of the solvent gave an orange oil (2.2 g), MH⁺ 174, NMR (CDCl₃): 1.2 (m, 2H), 1.45 (s, 9H), 1.6 (m, 1H), 1.75 (bd, 2H), 2.35 (s, 3H), 2.65 (bt, 2H), 2.8 (d, 2H), 4.1 (m, 2H). This material was used without further purification.

### Step 2 Preparation of N-Boc-piperidin-4-ylmethylthiol

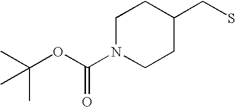

Sodium borohydride (2.2 g) was added in portions over 10 minutes to a solution of (N-Boc-piperidin-4-yl)methylthioacetate (2.2 g) in methanol (40 ml) at 0° C. The mixture was allowed to warm to room temperature and was stirred for 2 hours. The reaction mixture was evaporated and the residue was dissolved in water (10 ml), citric acid (2 g) was added and the mixture was extracted with dichloromethane (3×20 ml) and dried. Removal of the solvent gave the product as an orange oil, which by NMR contained ˜29% of the starting material. This product was used without further purification.

### Step 3 Preparation of [(N-Boc-piperidin-4-yl)methyl]-(4-methanesulphonyl-phenylmethyl)sulphide

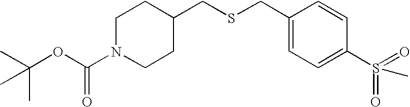

N-Boc-piperidin-4-ylmethylthiol (1.155 g) was added to a suspension of sodium hydride (200 mg of a 60% dispersion in mineral oil) in DMF at 0° C. and the mixture was stirred for 30 minutes. 4-Methanesulphonylbenzyl chloride (1.023 g) was added, the reaction mixture was allowed to warm to room temperature and was stirred for 1 hour. The reaction mixture was evaporated to dryness and the residue was dissolved in dichloromethane (30 ml) and washed with water (25 ml) and brine (25 ml) and dried. The solvent was evaporated and the residue was purified on a 50 g silica Bond Elut eluting with a solvent gradient (isohexane—50% ethyl acetatelisohexane). Yield 1 g, MH⁺ 300. NMR (CDCl₃): 1.2 (m, 2H), 1.45 (s, 9H), 1.5 (m, 1H), 1.8 (m, 2H), 2.35 (d, 2H), 2.65 (bt, 2H), 3.05 (s, 3H), 3.75 (s, 3H), 4.1 (m, 2H), 7.5 (d, 2H), 7.9 ( d, 2H).

### Step 4 Preparation of [(N-Boc-piperidin-4-yl)methyl]-(4-methanesulphonyl-phenylmethyl)sulphone

This was carried out as described in Step 2 of Method I. MH⁺ 376 (−tert-Butyl): NMR (CDCl₃): 1.3 (m,2H), 1.4 (s, 9H), 1.9 (m, 2H), 2.2 (m, 1H), 2.7-2.9 (m, 4H), 3.1 (s, 3H), 4.1 (m, 2H), 4.3 (s, 2H), 7.6 (d, 2H), 8.0 (d, 2H).

### Step 5 [(piperidin-4-yl)methyl]-(4-methanesulphonylphenylmethyl)sulphone

This was carried out as described in Step 3 of Method I, MH⁺ 332.

The following compounds were also prepared using a process analogous to Method J.

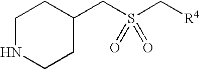

| R | MH⁺ |
|---|---|
| 4-cyanophenyl | 279 |
| 6-trifluoromethylpyridin-3-yl | 323 |
| pyridin-2-yl | 255 |
| pyridin-4-yl | 255 |

Method K

### 4-(2-[Piperidin-4yl]ethylsulphonyl)benzyl methyl sulphone

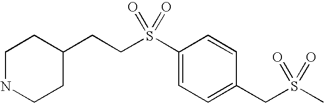

### Step 1 Preparation of 4-(2-[N-Boc-piperidin-4-yl]ethylsulphonyl)benzyl alcohol

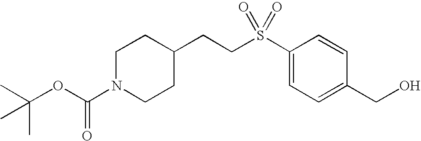

Lithium aluminium hydride (2.823 ml of a 1M solution in THF) was added dropwise to a solution of ethyl 4-(2-[N-Boc-piperidin-4-yl]ethylsulphonyl)benzoate (1.2 g) [prepared according to Method B using ethyl-4-mercaptobenzoate (CAS 28276-32-6) as starting material] in THF (20 ml) at 0° C. and the mixture was stirred for 30 minutes. Ethyl acetate (10 ml) was added followed by water (0.1 ml), 2M NaOH (0.1 ml) and water (1 ml) and Celite (2 g). The mixture was stirred for 5 minutes and filtered. The filtrate was evaporated to dryness to give 1.086 g, NMR (CDCl₃): 1.0-1.1 (m, 2H), 1.4 (s, 9H), 1.55-1.7 (m, 5H), 2.6 (bt, 2H), 3.1 (m, 2H), 4.1 (m, 2H), 4.8 (s, 2H), 7.6 (d, 2H), 7.9 (d, 2H).

### Step 2 Preparation of 4-(2-[N-Boc-piperidin-4-yl]ethylsulphonyl)benzyl alcohol tosylate

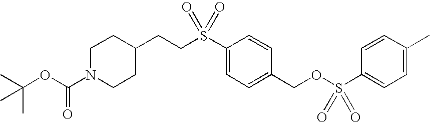

p-Toluenesulphonyl chloride (541 mg) was added to a solution of 4-(2-[N-Boc-piperidin-4-yl]ethylsulphonyl)benzyl alcohol (1.086 g) and triethylamine (0.473 ml) in dichloromethane (30 ml) at 0° C. The reaction mixture was allowed to warm to room temperature and was stirred for 16 hours. The reaction mixture was washed with water (25 ml), dried and evaporated to dryness. The residue obtained on evaporation of the solvent was passed through a 50 g silica Bond Elut column eluting with a solvent gradient (isohexane—50% ethyl acetate/isohexane), yield 765 mg. LC-MS showed this to be a mixture of the required tosylate and the chloro analogue. The mixture was used for the next step.

### Step 3 Preparation of 4-(2-[N-Boc-piperidin-4-yl]ethylsulphonyl)benzyl methyl thioether

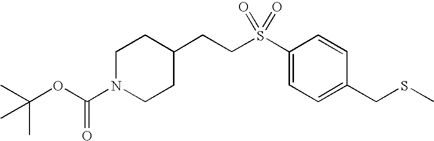

The chloride/tosylate mixture from Step 2 was added to a solution of the sodium salt of methanethiol in DMF at 0° C. The reaction mixture was allowed to warm to room temperature and was stirred for 16 hours then evaporated to dryness. The residue was dissolved in dichloromethane (20 ml) and washed with water (20 ml) and brine (20 ml) and dried. Evaporation of the solvent gave the product, yield 602 mg, MH⁺ 314.

### Step 4 Preparation of 4-(2-[N-Boc-piperidin-4-yl]ethylsulphonyl)benzyl methyl sulphone

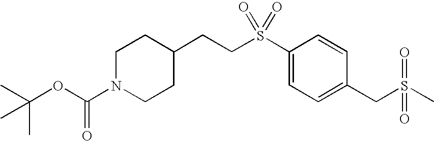

m-Chloroperbenzoic acid (720 mg) was added to a solution of the thioether (Step 3) in dichloromethane (20 ml) at 0° C. The reaction mixture was allowed to warm to room temperature and was stirred for 16 hours then washed with 2M NaOH (20 ml) and brine (20 ml) and dried. The residue obtained on evaporation of the solvent was passed through a 50 g silica Bond Elut column eluting with a solvent gradient (isohexane—50% ethyl acetate/isohexane), yield 416 mg, MH⁺ 390 (−tert-Butyl). NMR (CDCl₃): 1.0-1.2 (m, 2H), 1.4 (s, 9H), 1.5 (m, 1H), 1.6 (m,2H), 1.7 (m, 2H), 2.6 (bt, 2H), 2.85 (s, 3H), 3.1 (m, 2H), 4.1 (m, 2H), 4.3 (m, 2H), 7.8 (d, 2H), 7.95 (d, 2H).

### Step 5 Preparation of the Title Compound

4-(2-[N-Boc-piperidin-4-yl]ethylsulphonyl)benzyl methyl sulphone (402 mg) was stirred in 4M HCl in dioxane (10 ml) for 1 hour then diethylether was added and the precipitated solid was filtered and dried, yield (HCl salt) 375 mg. MH⁺ 346.

Method L

4-(2-[Piperidin-4-yl]ethylsulphonyl)benzamide

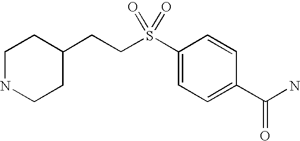

Step 1 Preparation of 4-(2-[N-Boc-piperidin-4-yl]ethylsulphonyl)benzamide

A mixture of ethyl 4-(2-[N-Boc-piperidin-4-yl]ethylsulphonyl)benzoate (0.8 g) [prepared according to Method B using ethyl-4-mercaptobenzoate (CAS 28276-32-6) as starting material] in methanolic ammonia (10 ml of 7M ammonia in methanol) was heated to 50° C. to give a clear solution and was allowed to stand at room temperature for 72 hours. The reaction mixture was evaporated to dryness and the residue obtained on evaporation of the solvent was passed through a 50 g silica Bond Elut column eluting with a solvent gradient (isohexane—50% ethyl acetate/isohexane), yield 394 mg, MH⁺ 297 (−Boc).

### Step 2 Preparation of Title Compound

4-(2-[N-Boc-piperidin-4-yl]ethylsulphonyl)benzamide (394 mg) was stirred in 4M HCl in dioxane (10 ml) for 1 hour then diethylether was added and the precipitated solid was filtered and dried, yield (HCl salt) 428 mg. MH⁺ 297.

Method M

### Preparation of {4-[(2-piperidin-4-ylethyl)sulfonyl]phenoxy}acetonitrile

### Step 1 tert-Butyl 4-(2-{[4-(cyanomethoxy)phenyl]sulfonyl}ethyl)piperidine-1-carboxylate

Bromoacetonitrile (320 mg) was added to a solution of N-tert-butoxycarbonyl-4-[2-(4-hydroxyphenylsulphonyl)ethyl]piperidine (1 g) in acetone (20 ml) containing potassium carbonate (037 g) and the mixture was stirred at room temperature for 16 hours. The reaction mixture was filtered and the filtrate was evaporated to dryness. The residue was dissolved in ethyl acetate (50 ml) and the solution was washed with water (2×50 ml), dried and evaporated to dryness, yield 1.4 g. NMR (CDCl₃): 1.1 (m, 2H), 1.42 (s, 9H), 1.6 (m, 5H), 2.6 (t, 3H), 3.1 (m, 2H), 4.1 (m, 2H), 4.9 (s, 2H), 7.1 (d, 2H), 7.9 (d, 2H).

### Step 2 4-(2-{[4-(Cyanomethoxy)phenyl]sulfonyl}ethyl)piperidine

tert-Butyl 4(2-{[4-(cyanomethoxy)phenyl]sulfonyl}ethyl)piperidine-1-carboxylate (1.4 g) was dissolved in dioxane (5 ml) and HCl/dioxane (20 ml of 4M solution) was added. The mixture was stirred for 1 hour and diethyl ether was added (75 ml) and the oily precpitate obtained was triturated to give {4-[(2-piperidin-4-ylethyl)sulfonyl]phenoxy}acetonitrile hydrochloride, yield 0.9 g. M⁺H 309.

Method N

### Preparation of 4-(2-{[4-(2-methyl-2H-tetrazol-5-yl)phenyl]sulfonyl}ethyl)piperidine

### Step 1 Benzyl 4-{2-[(4-cyanophenyl)sulfonyl]ethyl}piperidine-1-carboxylate

Benzyl chloroformate (800 mg) was added to a solution of 4-[(2-piperidin-4-ylethyl)sulfonyl]benzonitrile (1.4 g, Method B) and triethylamine (1.3 g) in dichloromethane (25 ml) at 0° C. The reaction mixture was allowed to warm to room temperature and was stirred for 2 hours, washed with 2M HCl (2×20 ml) and 2M NaOH (2×20 ml) and dried. The residue obtained on removal of the solvent was purified by chromatography on a Bond-Elut column using an eluant gradient of hexane—50% ethyl acetate/hexane. Yield 1.4 g. NMR (CDCl₃): 0.9 (m, 1H), 1.1 (m, 1H), 1.8 (m, 4H), 2.7 (m, 2H), 3.1 (m, 2H), 4.2 (m, 2H), 5.1 (s, 2H), 7.3 (m, 5H), 7.9 (d, 2H), 8.05 (d, 2H).

### Step 2 Benzyl-4-(2-{[4-(1H-tetrazol-5-yl)phenyl]sulfonyl}ethyl)piperidine-1-carboxylate

The product from step 1 was mixed with sodium azide (220 mg) and ammonium chloride (182 mg) in DMF (25 ml) and heated at 100° C. for 4 hours. The reaction mixture was allowed to cool and the solvent was evaporated. The residue was dissolved in dichloromethane (50 ml) and washed with 2M NaOH (2×20 ml) and dried. Removal of the solvent gave the product, yield 1.9 g, M⁺H 456, which was used directly in the next stage.

### Step 3 Preparation of benzyl 4-(2-{[4-(2-methyl-2H-tetrazol-5-yl)phenyl]sulfonyl}ethyl)-piperidine-1-carboxylate and benzyl 4-(2-{[4-(1-methyl-1H-tetrazol-5-yl)phenyl]sulfonyl}-ethyl)piperidine-1-carboxylate

Methyl iodide (710 mg) was added to a solution of the product from step 2 (1.9 g) in ethanol (25 ml) containing 2M NaOH (5 ml) and the mixture was stirred for 16 hours. A second portion of methyl iodide (710 mg) and 2M NaOH (5 ml) was added and stirring was continued for 72 hours. The reaction mixture was concentrated and water (30 ml) added. The precipitated solid was collected, dried and dissolved in dichloromethane and passed through a Bond-elut column eluting with 60% ethyl acetate in hexane to give:

Benzyl 4-(2-{[4-(2-methyl-2H-tetrazol-5-yl)phenyl]sulfonyl}ethyl)piperidine-1-carboxylate, yield 800 mg, NMR (CDCl₃): 1.1(m, 2H), 1.8 (m, 5H), 2.7 (m, 2H), 3.1 (m, 2H), 4.1 (m, 2H), 4.4 (s, 3H), 5.1 (s, 2H), 7.3 (m, 5H), 8.05 (d, 2H), 8.35 (d, 2H). M⁺H470.

Benzyl 4(2-{[4-(1-methyl-1H-tetrazol-5-yl)phenyl]sulfonyl}ethyl)piperidine-1-carboxylate, yield 200 mg, NMR (CDCl₃): 1.1(m, 2H), 1.8 (m, 5H), 2.7 (m, 2H), 3.1 (m, 2H), 4.1 (m, 2H), 4.15 (s, 3H), 5.1 (s, 2H), 7.4 (m, 5H), 7.95 (d, 2H), 8.15 (d, 2H). M⁺H 470.

### Step 4 Preparation of 4-(2-{[4-(2-methyl-2H-tetrazol-5-yl)phenyl]sulfonyl}ethyl)piperidine

20% Palladium hydroxide on charcoal was added to a solution of benzyl 4-(2-{[4-(2-methyl-2H-tetrazol-5-yl)phenyl]sulfonyl}ethyl)piperidine-1-carboxylate (700 mg) in a mixture of ethyl acetate (50 ml) and ethanol (150 ml) and the mixture was hydrogenated under a hydrogen filled balloon. The catalyst was filtered and the filtrate evaporated to dryness to give the title compound as a white solid, yield 600 mg. NMR (CDCl₃): 1.1 (m, 2H), 1.7 (m, 5H), 2.6 (m, 2H), 3.05 (m, 2H), 3.15 (m, 2H), 4.4 (s, 3H), 8.0 (d, 2H), 8.39 (d, 2H).

The corresponding 1-methyl analogue was prepared in an analogous manner starting with benzyl 4-(2-{[4-(1-methyl-1H-tetrazol-5-yl)phenyl]sulfonyl}ethyl)piperidine-1-carboxylate, yield 120 mg.

Method O

### Preparation of 4-[(2-piperidin-4-ylethyl)sulfonyl]aniline

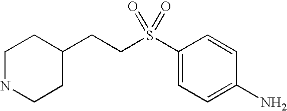

### Step 1 tert-Butyl 4-{2-[(4-aminophenyl)sulfonyl]ethyl}piperidine-1-carboxylate

Nickel (II) acetate tetrahydrate (45 mg) was added to borohydride exchange resin (borohydride on Amberlite® IRA-140 [available from Aldrich]) (3.61 g) in methanol (35 ml) and after the reaction had subsided was allowed to stand for 1 minute. A solution of tert-butyl 4-{2-[(4-nitrophenyl)sulfonyl]ethyl}piperidine-1-carboxylate (717 mg) [prepared according to Method B] in methanol (5 ml) was added and the mixture was stirred at room temperature for 16 hours. The reaction mixture was filtered through Celite® and the resin was washed with methanol (3×10 ml). The combined filtrate and washings were evaporated to dryness and the product was used without further purification. LC/MS, M+H 269 (product −Boc group).

### Step 2 Preparation of Title Compound

The product from step 1 (450 mg) was dissolved in 4M HCl in dioxane (10 ml) and allowed to stand for 30 minutes. Diethyl ether (20 ml) was added and a solid was obtained on trituration, yield 597 mg, M+H 269.

Method P

### Preparation of N-{4-[(2-piperidin-4-ylethyl)sulfonyl]phenyl}methanesulfonamide

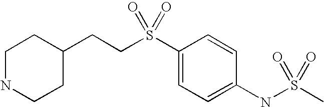

### Step 1 Preparation of tert-butyl 4-[2-({4-[(methylsulfonyl)amino]phenyl}thio)ethyl]-piperidine-1-carboxylate

Methanesulphonyl chloride (0.63 ml) was added to a solution of tert-butyl 4-{2-[(4-aminophenyl)thio]ethyl}piperidine-1-carboxylate (1.61 g, Method B) in pyridine (40 ml) at 0° C. and allowed to warm to room temperature. The reaction mixture was stirred for 5 hours then evaporated to dryness. The residue was dissolved in dichloromethane (40 ml) washed with water (2×20 ml) and dried. The residue was purified by chromatography on a 50 g silica Bond-elut column using an eluant gradient of hexane—50% ethyl acetate/hexane. Yield 320 mg. M+H 413.

### Step 2 Preparation of tert-butyl 4-[2-({4-[(methylsulfonyl)amino]phenyl}sulfonyl)ethyl]-piperidine-1-carboxylate

m-Chloroperbenzoic acid (375 mg) was added to a solution of the product from step 1 (314 mg) in dichloromethane (30 ml) at 0° C. and was stirred for 3 hours. The reaction mixture was washed with aqueous sodium bicarbonate (20 ml), brine (20 ml) and dried. Removal of the solvent gave tert-butyl 4-[2-({4-[(methylsulfonyl)amino]phenyl}sulfonyl)ethyl]piperidine-1-carboxylate, 330 mg, M+H 347.

### Step 3 Preparation of Title Compound

The tert-butoxycarbonyl group was removed using the procedure described in step 2 of Method O to give the title compound as the hydrochloride salt, M+H 347.

Method Q

### Preparation of N-phenyl-N′-{4-[(2-piperidin-4-ylethyl)sulfonyl]phenyl}urea

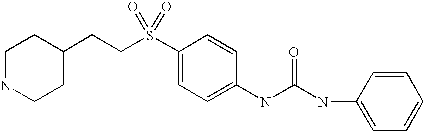

Phenylisocyanate (86 μl) was added to a solution of tert-butyl 4-{2-[(4-aminophenyl)sulfonyl]ethyl}piperidine-1-carboxylate (300 mg, Method O) in dichloromethane (10 ml) and the mixture was stirred for 16 hours. A further equivalent of phenylisocyanate was added and stirring continued for 24 hours. The reaction mixture was poured onto a 20 g silica Bond-elut column and eluted with a solvent gradient of hexane—70% ethyl acetate/hexane. M⁺H 388 (M-Boc).

The tert-butoxycarbonyl group was removed using the procedure described in step 2 of Method O to give the title compound as the hydrochloride salt, yield 124 mg, M+H 388.

Method R

### Preparation of (3R)-3-(3,5-difluorophenyl)-3-[4-(methylsulfonyl)phenyl]propanoic acid

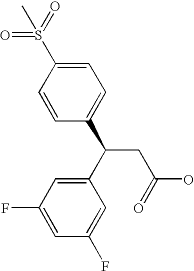

To a stirred solution of (4S,5R)-1-{(3R)-3-(3,5-difluorophenyl)-3-[4-(methylsulfonyl)phenyl]propanoyl}-3,4-dimethyl-5-phenylimidazolidin-2-one (7.5 g) (prepared according to Method A, step 1, using 3,5-difluorphenylmagnesium bromide) in THF (300 ml) was added a solution of lithium hydroxide monohydrate(2.0 g) in water (30 ml). After stirring for 16 hours at 20-25° C., the solution was evaporated at reduced pressure and the residue partitioned between water (200 ml) and dichloromethane (200 ml). The aqueous layer was separated and washed again with dichloromethane, then acidified to pH 2 with 2N HCl and the precipitate extracted into ethyl acetate (200 ml) which was dried over magnesium sulphate and evaporated to give 4.8 gm pale cream solid (96% yield), NMR: 3.10 (m, 2H), 3.15 (s, 3H), 4.60 (dd, 1H), 7.02 (t, 1H), 7.18 (d, 2H), 7.67 (d, 2H), 7.82 (d, 2H).

Method S

### Preparation of (3R)-3-(3,5-difluorophenyl)-N-methoxy-N-methyl-3-[4-(methylsulfonyl)phenyl]propanamide

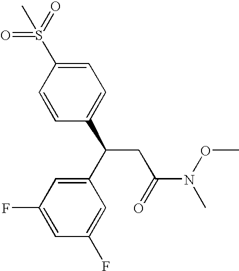

To a stirred mixture of (3R)-3-(3,5-difluorophenyl)-3-[4-(methylsulfonyl)phenyl]-propanoic acid (4.8 g), N,O-dimethyl hydroxylamine hydrochloride (1.5 g) and HATU (1.5 g) in dichloromethane (200 ml) was added DIPEA (10 ml) and stirring was continued for 16 hours at 20-25° C.; water (100 ml) was added and the organic layer separated, then washed successively with 1M HCl, 1M NaOH and water. The solution was dried (MgSO₄), evaporated and the residue purified by chromatography on silica, eluting with ethyl acetate to give 4.7 g (gum) 87% yield. NMR (CDCl₃): 3.04 (s, 3H), 3.13 (s, 3H), 3.18 (d, 2H), 3.65 (s, 3H), 4.76 (t, 1H), 6.67 (t, 1H), 6.78 (d, 2H), 7.44 (d, 2H), 7.89 (d, 2H).

Method T

### Preparation of (4R)-4-(3,5-difluorophenyl)-4-[4-(methylsulfonyl)phenyl]butan-2-one

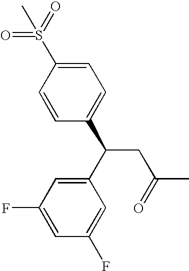

To a stirred solution of (3R)-3-(3,5-difluorophenyl)-N-methoxy-N-methyl-3-[4-(methylsulfonyl)phenyl]propanamide (4.7 g) in dry THF (50 ml)under argon, cooled to −20° C., was added 12.0 ml 3M methyl magnesium bromide (12 ml of a 3M solution in ether). The reaction was stirred for a further 1 hour at 0° C., then 50 ml 1M HCl was cautiously added and the mixture extracted with ethyl acetate, dried and evaporated to give 4.1 gm (gum) 99% yield. NMR (CDCl₃): 2.16 (s, 3H), 3.043 (s, 3H), 3.21 (d, 2H), 4.69 (t, 1H), 6.67 (t, 1H), 6.77 (d, 2H), 7.41 (d, 2H), 7.89 (d, 2H).

Method U

### (S) N-(3-Amino-3-phenylpropyl)-4-[2-(4-methanesulphonylphenylsulphonyl)ethyl]piperidine

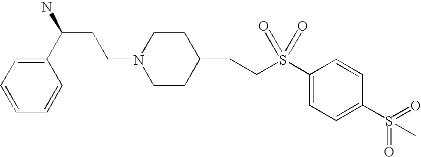

Sodium triacetoxyborohydride (1.6 g) was added to a mixture of (S) 3-phenyl-3-(tert-butoxycarbonylamino)propionaldehyde (1.23 g) and 4-[2-(4-methanesulphonylphenyl-sulphonyl)ethyl]piperidine hydrochloride (1.215 g) (Method B) in dichloromethane (50 ml) and the mixture was stirred for 16 hours. The reaction mixture was washed successively with 2M sodium hydroxide (15 ml), water (15 ml) and brine (15 ml) and dried. The dichloromethane solution was stirred with PS—NCO (isocyanate resin, 1.5 g) for 16 hours and filtered. The filtrate was chromatographed on a 50 g silica Bond Elut column eluting with ethyl acetate to give the Boc protected title compound as a white solid, yield 1.595 g, MH⁺ 565.

The Boc protected compound (1.59 g) was dissolved in 4M HCl/dioxane (10 ml) and allowed to stand at room temperature for 1 hour. The reaction mixture was evaporated to dryness, redissolved in 2M sodium hydroxide (10 ml) and extracted with dichloromethane (2×20 ml) and dried. Removal of the solvent gave the title compound, yield 0.56 g, MH⁺ 465.

### (S) 3-phenyl-3-(tert-butoxycarbonylamino)propionaldehyde

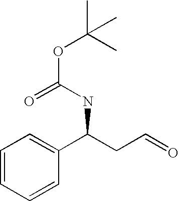

Lithium aluminium hydride (19 ml of 1M solution in THF) was added to a solution of (S) 3-phenyl-3-(tert-butoxycarbonylamino)propionic acid (5.01 g) in THF (50 ml) at 0° C. The reaction mixture was stirred for 1 hour and ethyl acetate (20 ml) was added followed by water (0.5 ml), 6M sodium hydroxide (0.5 ml) and water (5 ml). The mixture was filtered through Celite and evaporated to dryness to give (S) 3-phenyl-3-(tert-butoxycarbonylamino)propanol, yield 2.89 g. This material was dissolved in dichloromethane (40 ml) and Dess Martin periodinane (2.12 g) was added. The reaction mixture was stirred for 1 hour then washed with 2M sodium hydroxide (2×20 ml) and brine (10 ml) and dried. The dichloromethane solution was concentrated to a volume of about 20 ml and used directly in the next stage.

### EXAMPLE 19

The ability of compounds to inhibit the binding of RANTES was assessed by an in vitro radioligand binding assay. Membranes were prepared from Chinese hamster ovary cells which expressed the recombinant human CCR5 receptor. These membranes were incubated with 0.1 nM iodinated RANTES, scintillation proximity beads and various concentrations of the compounds of the invention in 96-well plates. The amount of iodinated RANTES bound to the receptor was determined by scintillation counting. Competition curves were obtained for compounds and the concentration of compound which displaced 50% of bound iodinated RANTES was calculated (IC₅₀). Certain compounds of formula (I) have an IS₅₀ of less than 50 μM.

### EXAMPLE 20

The ability of compounds to inhibit the binding of MIP-1α was assessed by an in vitro radioligand binding assay. Membranes were prepared from Chinese hamster ovary cells which expressed the recombinant human CCR5 receptor. These membranes were incubated with 0.1 nM iodinated MIP-1α, scintillation proximity beads and various concentrations of the compounds of the invention in 96-well plates. The amount of iodinated MIP-1α bound to the receptor was determined by scintillation counting. Competition curves were obtained for compounds and the concentration of compound which displaced 50% of bound iodinated MIP-1α was calculated (IC₅₀). Certain compounds of formula (I) have an IC₅₀ of less than 50 μM.

Results from this test for certain compounds of the invention are presented in Table XV. In Table XV the results are presented as Pic50 values. A Pic50 value is the negative log (to base 10) of the IC₅₀ result, so an IC50 of 1 μM (that is 1×10⁻⁶M) gives a Pic50 of 6. If a compound was tested more than once then the data below is an average of the probative tests results.

TABLE XV

| Table Number | Compound number | Pic50 |
|---|---|---|
| I | 6 | 6.91 |
| I | 8 | 8.58 |
| I | 13 | 7.9 |
| I | 16 | 8.63 |
| III | 1 | 8.8 |
| III | 31 | 9.0 |
| IV | 2 | 8.8 |
| V | 7 | 9.2 |
| V | 19 | 8.7 |
| V | 26 | 8.85 |
| VIII | 1 | 8.95 |
| XI | 18 | 9.3 |

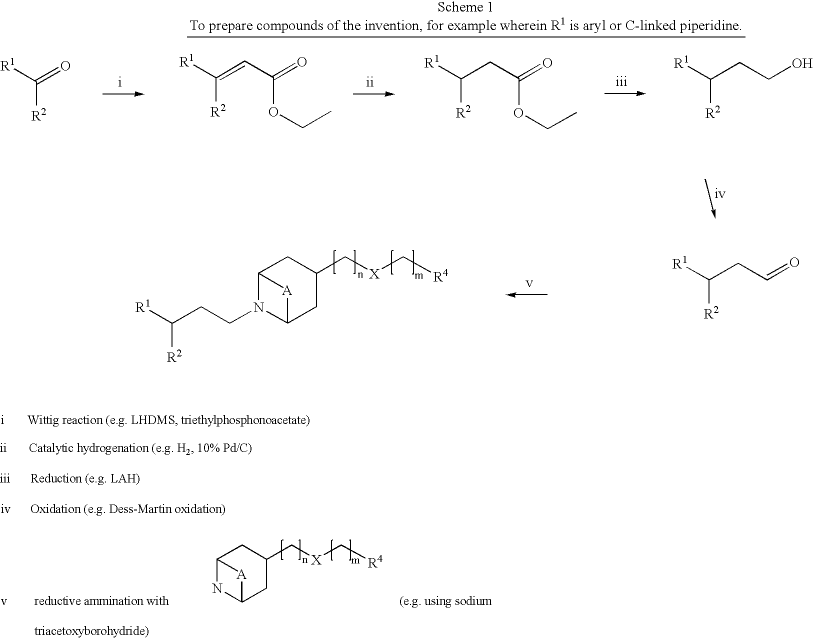 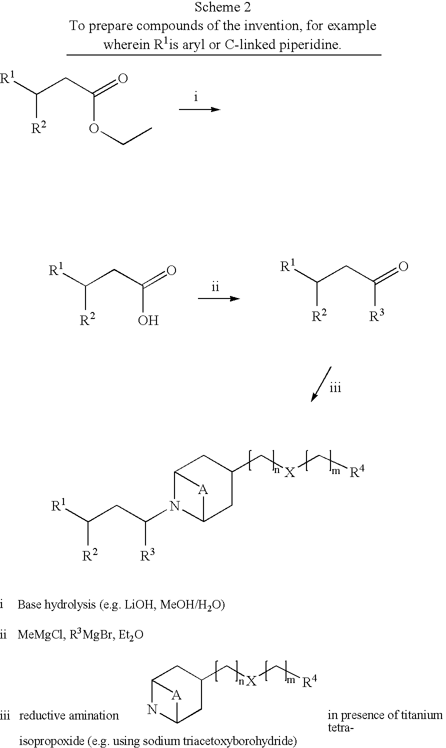 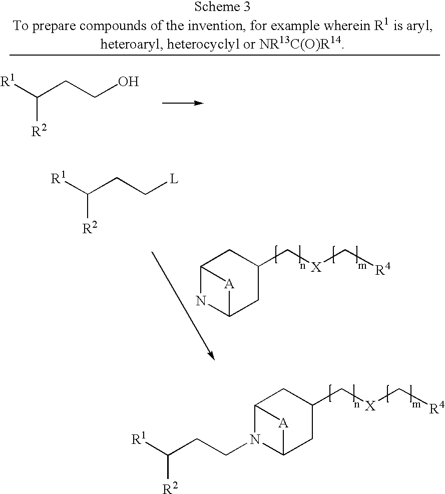 in which L is an activated group, such as halogen, mesylate, tosylate or triflate. 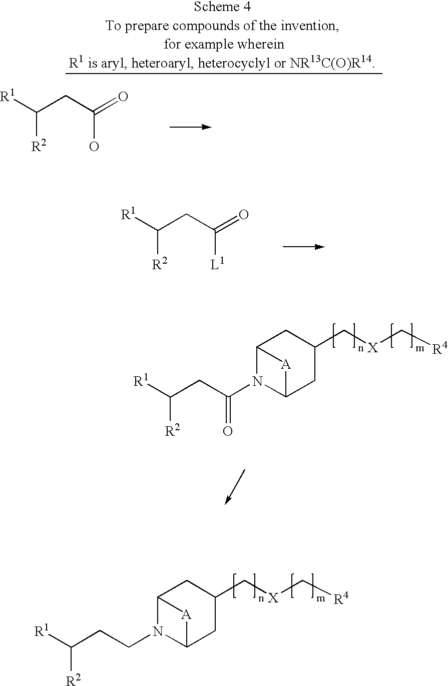
in which L¹ is a halogen, an activated ester or a complex formed with a carbodiimide.

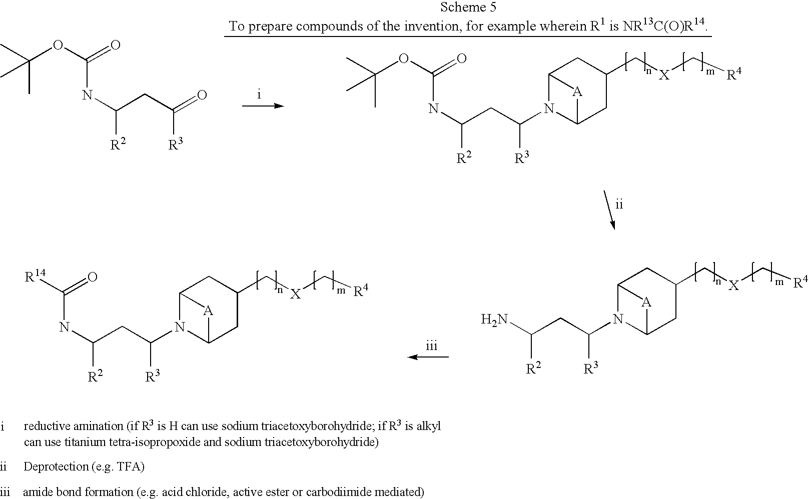

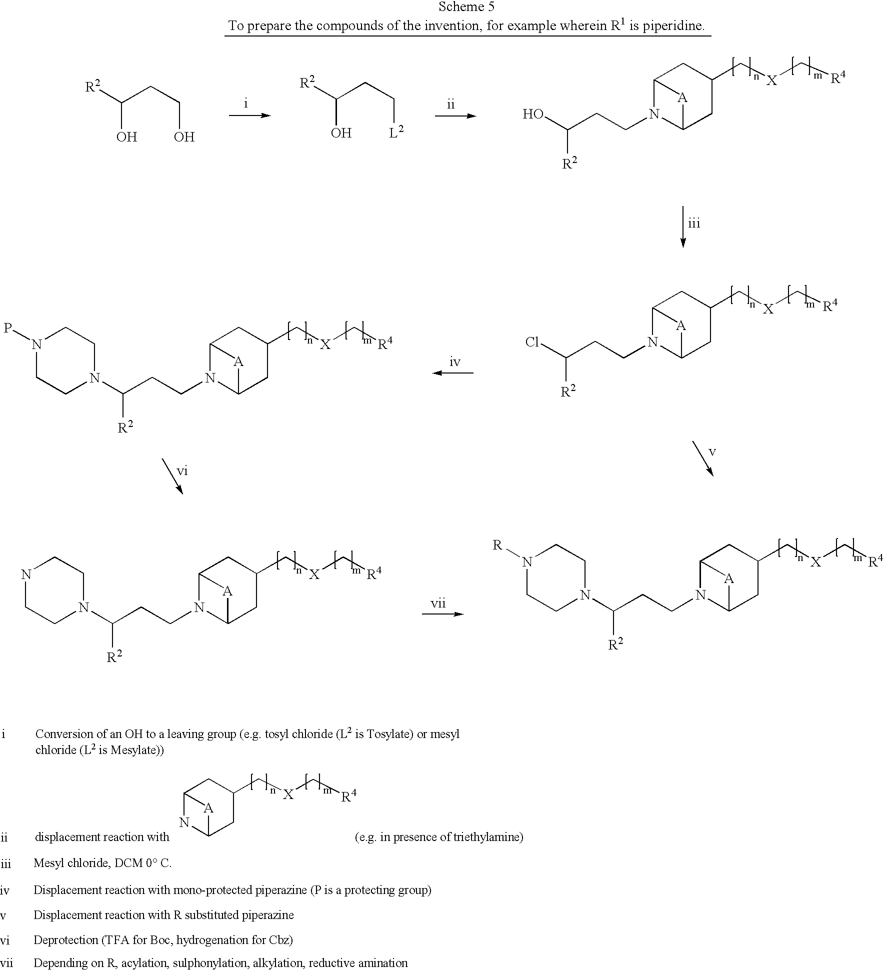

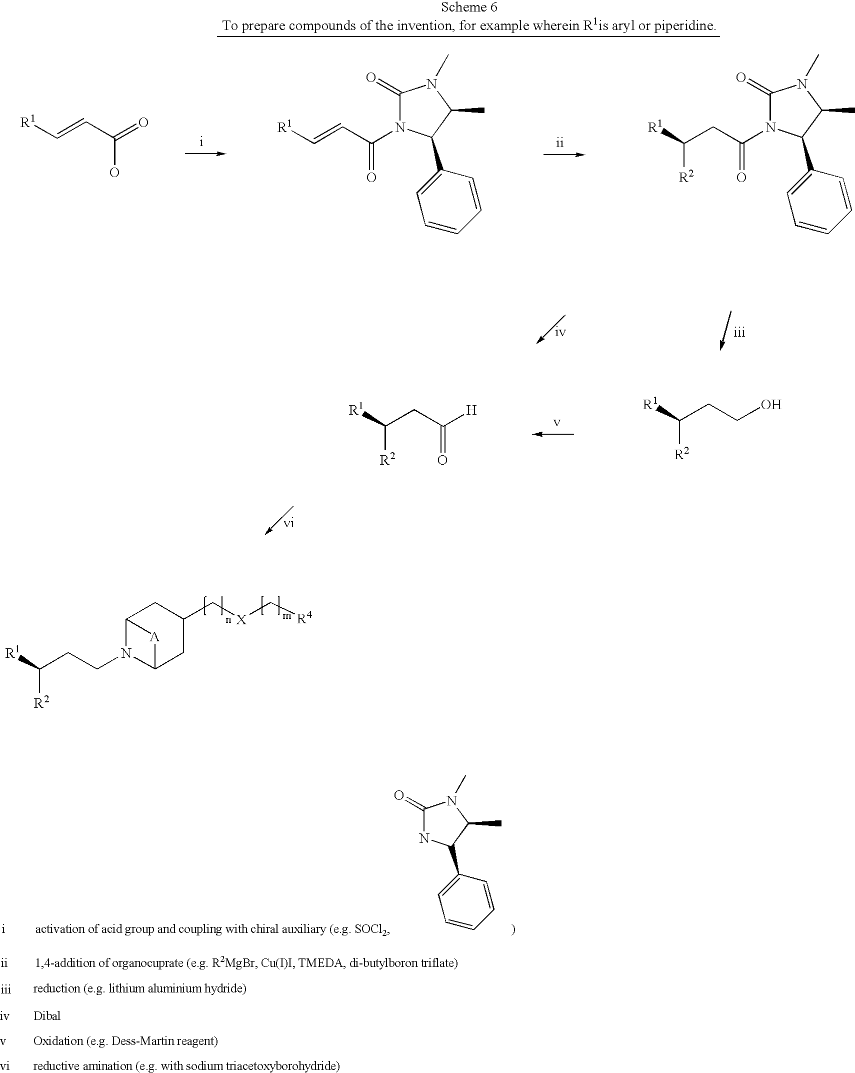

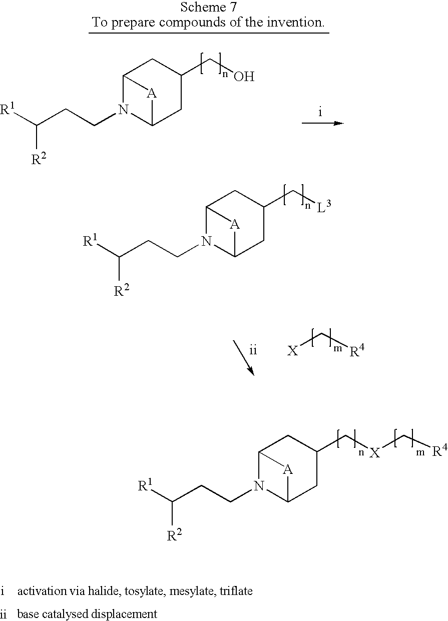

1. A compound of formula (I): 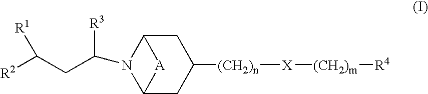
wherein:
A is absent or is (CH₂)₂;
R¹ is C₁₋₈ alkyl, C(O)NR¹⁰R¹¹, C(O)₂R¹², NR¹³C(O)R¹⁴, NR¹⁵C(O)NR¹⁶R¹⁷, NR¹⁸C(O)₂R¹⁹, heterocyclyl, aryl or heteroaryl;
R¹⁰, R¹³, R¹⁵, R¹⁶ and R¹⁸ are hydrogen or C₁₋₆ alkyl;
R¹¹, R¹², R¹⁴, R¹⁷ and R¹⁹ are C₁₋₈ alkyl (optionally substituted by halo, hydroxy, C₁₋₆ alkoxy, C₁₋₆ haloalkoxy, C₃₋₆ cycloalkyl (optionally substituted by halo), C₅₋₆ cycloalkenyl, S(C₁₋₄ alkyl), S(O)(C₁₋₄ alkyl), S(O)₂(C₁₋₄ alkyl), heteroaryl, aryl, heteroaryloxy or aryloxy), aryl, heteroaryl, C₃₋₇ cycloalkyl (optionally substituted by halo or C₁₋₄ alkyl), C₄₋₇ cycloalkyl fused to a phenyl ring, C₅₋₇ cycloalkenyl, or, heterocyclyl (itself optionally substituted by oxo, C(O)(C₁₋₆ alkyl), S(O)k(C₁₋₆ alkyl), halo or C₁₋₄ alkyl); or R¹¹, R¹², R¹⁴ and R¹⁷ can also be hydrogen;
or R¹⁰ and R¹¹, and/or R¹⁶ and R¹⁷ may join to form a 4-, 5- or 6-membered ring which optionally includes a nitrogen, oxygen or sulphur atom, said ring being optionally substituted by C₁₋₆ alkyl, S(O)₁(C₁₋₆ alkyl) or C(O)(C₁₋₆ alkyl);
R² is C₁₋₆ alkyl, phenyl, heteroaryl or C₃₋₇ cycloalkyl;
R³is H or C₁₋₄ alkyl;
R⁴is aryl, heteroaryl, C₁₋₆ alkyl or C₃₋₇ cycloalkyl;
X is O or S(O)ₚ;
m and n are, independently, 0, 1, 2 or 3, provided m+n is 1 or more;
aryl, phenyl and heteroaryl moieties are independently optionally substituted by one or more of halo, cyano, nitro, hydroxy, OC(O)NR²⁰R²¹, NR²²R²³, NR²⁴C(O)R²⁵, NR²⁶C(O)NR²⁷R²⁸, S(O)₂NR²⁹R³⁰, NR³¹S(O)₂R³², C(O)NR³³R³⁴, CO₂R³⁶, NR³⁷CO₂R³⁸, S(O)_{q}R³⁹, OS(O)₂R⁴⁹, C₁₋₆ alkyl (optionally mono-substituted by S(O)₂R⁵⁰ or C(O)NR⁵¹R⁵²), C₂₋₆ alkenyl, C₂₋₆ alkynyl, C₃₋₁₀ cycloalkyl, C₁₋₆ haloalkyl, C₁₋₆ alkoxy(C₁₋₆)alkyl, C₁₋₆ alkoxy (optionally mono-substituted by CO₂R⁵³, C(O)NR⁵⁴R⁵⁵, cyano, heteroaryl or C(O)NHS(O)₂R⁵⁶), NHC(O)NHR⁵⁷, C₁₋₆ haloalkoxy, phenyl, phenyl(C₁₋₄)alkyl, phenoxy, phenylthio, phenylS(O), phenylS(O)₂, phenyl(C₁₋₄)alkoxy, heteroaryl, heteroaryl(C₁₋₄)alkyl, heteroaryloxy or heteroaryl(C₁₋₄)alkoxy; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃;
unless otherwise stated heterocyclyl is optionally substituted by C₁₋₆ alkyl [optionally substituted by phenyl {which itself optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)} or heteroaryl {which itself optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}], phenyl {optionally substituted by halo, C₁₋₄ alkyl, C₁₋₄ alkoxy, cyano, nitro, CF₃, OCF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, heteroaryl {optionally substituted by halo, C₁₋₄ alkyl, C₁₋₄ alkoxy, cyano, nitro, CF₃, (C₁₋₄ alkyl)C(O)NH, S(O)₂NH₂, C₁₋₄ alkylthio, S(O)(C₁₋₄ alkyl) or S(O)₂(C₁₋₄ alkyl)}, S(O)₂NR⁴⁰R⁴¹, C(O)R⁴², C(O)₂(C₁₋₆ alkyl), C(O)₂(phenyl(C₁₋₂ alkyl)), C(O)NHR⁴³, S(O)₂R⁴⁴, NHS(O)₂NHR⁴⁵, NHC(O)R⁴⁶, NHC(O)NHR⁴⁷ or NHS(O)₂R⁴⁸, provided none of these last four substituents is linked to a ring nitrogen;
k, l, p and q are, independently, 0, 1 or 2;
R²⁰, R²², R²⁴, R²⁶, R²⁷, R²⁹, R³¹, R³³, R³⁷, R⁴⁰, R⁵¹ and R⁵⁴ are, independently, hydrogen or C₁₋₆ alkyl;
R²¹, R²³, R²⁵, R²⁸, R³⁰, R³², R³⁴, R³⁶, R³⁸, R³⁹, R⁴¹, R⁴², R⁴³, R⁴⁴, R⁴⁵, R⁴⁶, R⁴⁷, R⁴⁸, R⁴⁹, R⁵⁰, R⁵², R⁵³, R⁵⁵, R⁵⁶ and R⁵⁷ are, independently, C₁₋₆ alkyl (optionally substituted by halo, hydroxy, C₁₋₆ alkoxy, C₁₋₆ haloalkoxy, C₃₋₆ cycloalkyl, C₅₋₆ cycloalkenyl, S(C₁₋₄ alkyl), S(O)(C₁₋₄ alkyl), S(O)₂(C₁₋₄ alkyl), heteroaryl, phenyl, heteroaryloxy or phenyloxy), C₃₋₇ cycloalkyl, phenyl or heteroaryl; wherein any of the immediately foregoing phenyl and heteroaryl moieties are optionally substituted with halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; and
R²¹, R²³, R²⁵, R²⁸, R³⁰, R³⁴, R³⁶, R⁴¹, R⁴², R⁴³, R⁴⁵, R⁴⁶, R⁴⁷, R⁵², R⁵³, R⁵⁵ and R⁵⁷ may additionally be hydrogen;
or a pharmaceutically acceptable salt thereof or a solvate thereof.

2. A compound as claimed in claim 1 wherein R¹ is NHC(O)R¹⁴, phenyl or heterocyclyl, wherein R¹⁴ is as defined in claim 1, and phenyl and heterocyclyl are optionally substituted as described in claim 1.

3. A compound as claimed in claim 1, wherein R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃; wherein n is 0, 1 or 2.

4. A compound as claimed in claim 1, wherein R³ is hydrogen.

5. A compound as claimed in claim 1, wherein R⁴ is phenyl optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl, OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃.

6. A compound as claimed in claim 1, wherein A is absent.

7. A compound as claimed in claim 1, wherein n is 2.

8. A compound as claimed in claim 1, wherein m is 0.

9. A compound as claimed in claim 1, wherein X is S(O)₂.

10. A process for preparing of a compound as claimed in claim 1 comprising:
a. to prepare a compound wherein R³ is hydrogen, coupling a compound of formula (III): 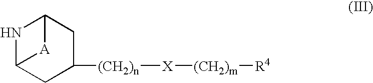
wherein R⁴, m, n, A and X are as defined in claim 1, with a compound of formula (IV): 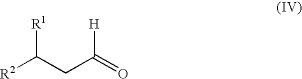
wherein R¹ and R² are as defined in claim 1, in the presence of NaBH(OAc)₃ (wherein Ac is C(O)CH₃) in a suitable solvent at room temperature;
b. to prepare a compound wherein R³ is hydrogen, coupling a compound of formula (III): 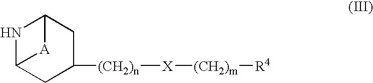
wherein R⁴, m, n, A and X are as defined in claim 1, with a compound of formula (V): 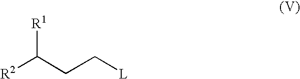
wherein R¹ and R² are as defined in claim 1 and L is a leaving group, in the presence of a base, in a suitable solvent at a temperature from 60° C. to the boiling point of the solvent.

11. A pharmaceutical composition which comprises a compound as claimed in claim 1, or a pharmaceutically acceptable salt thereof or solvate thereof, and a pharmaceutically acceptable adjuvant, diluent or carrier.

12. (canceled)

13. (canceled)

14. A method of treating a CCR5 mediated disease state comprising administering to a patient in need of such treatment an effective amount of a compound as claimed in claim 1, or a pharmaceutically acceptable salt thereof or solvate thereof.

15. A compound as claimed in claim 2, wherein R² is phenyl or heteroaryl, either of which is optionally substituted by halo, C₁₋₄ alkyl, C₁₋₄ alkoxy, S(O)ₙ(C₁₋₄ alkyl), nitro, cyano or CF₃; wherein n is 0, 1 or 2.

16. A compound as claimed in claim 2, wherein R³ is hydrogen.

17. A compound as claimed in claim 2, wherein R⁴ is phenyl optionally substituted by one or more of halo, hydroxy, nitro, S(C₁₋₆ alkyl), S(O)(C₁₋₆ alkyl), S(O)₂(C₁₋₆ alkyl), S(O)₂NH₂, S(O)₂NH(C₁₋₆ alkyl), S(O)₂N(C₁₋₆ alkyl)₂, cyano, C₁₋₆ alkyl, C₁₋₆ alkoxy, CH₂S(O)₂(C₁₋₆ alkyl), OS(O)₂(C₁₋₆ alkyl), OCH₂heteroaryl, OCH₂CO₂H, OCH₂CO₂(C₁₋₆ alkyl), OCH₂C(O)NH₂, OCH₂C(O)NH(C₁₋₆ alkyl), OCH₂CN, NH₂, NH(C₁₋₆ alkyl), N(C₁₋₆ alkyl)₂, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHC(O)O(C₁₋₆ alkyl), NHS(O)₂(C₁₋₆ alkyl), CF₃, CHF₂, CH₂F, CH₂CF₃, OCF₃, heteroaryl or heteroaryl(C₁₋₄ alkyl); wherein the foregoing heteroaryl groups are optionally substituted by halo, hydroxy, nitro, S(C₁₋₄ alkyl), S(O)(C₁₋₄ alkyl), S(O)₂(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), CF₃ or OCF₃.

18. A compound as claimed in claim 2, wherein A is absent.

19. A compound as claimed in claim 2, wherein n is 2.

20. A compound as claimed in claim 2, wherein m is 0.
